SEQUENCE LISTING (66 sequences)

SEQ ID NO: 1 - DNA, 1440 bp, Lactobacillus sake
atgacaaatcaactaacgactaacgaagggcaaccatgggcggacaatcaacattcacaaactgccggccaacgcggtcccgtcttaatccaagattatcaattactcgaaaaactcgcccattttaaccgcgaacgcattcctgaacgagtggttcacgccaaaggtgctggcgctaaaggttatttcaaggtcacaaaagacatgagcgcctacaccaaagctgctgtcttcagtggcgtcggcaaaaaaacaccgcttatcactcgtttttctcaagtcgctggtgaagctggctatcccgatacataccgcgatgttcgtggtttcgccgttaaattctacacggaagaaggcaattacgatattgttggcaataatacgccggtcttcttcgtcaatgatccactaaaattccccgatttcattcattcacaaaaacgtgatcctcggacacatgcccgtagccaagatatgcaatgggatttctggtccctttcacccgaatctgtccatcaagttacgattctcatgagtgaccgcgggattcctgccagttaccgaatgatgcacggctttggtagccacaccttcaaatgggtcaacgcacaaggtgaacaattctgggttaaatatcatttcaaaacgaaccaaggcgttcataatctcagcaacgaactcgccgatgaactcgctggtaaggatactgattaccttcaaaacgatttattcgacgcaattgaaacgggcgattatcccagttggactgtcgccgtccaactcgtcccttatgaagatggtttgaattatccccaagatatttttgatgttaccaaggttatttcacaaaaggattatccattaatcgaaatcggtcaaatggtcctcgatgaaaatccaacgaataacttcgaagatatcgaagaattggccttctcaccagctaacttagtccctggaattgaagcatcaccggacaaattactccaaggtcgactatttggttataaggatgcggaacgttaccgacttggtgccaactacgaacaactgccaatcaaccgaccaaaagtccctgttcataattacgaacgtgacggtgtcatggcccaaaaacaagaaacgggcgttaactacgaaccaaatagtcaagatggacccactgaagtcccagcagctaagattcatagtgatcaactctctggtacaactggcaacttctctaccgatcccgattattactcagcagctggcaaactttatcggctactatccgctgatgaacaaacccgtttaatcgaaaatattcgcatgaatcttggtcaagtaactaaaccagaaattcaaattcgcgaagttaaacaattttaccaagctgatccagaatacggtcgacgcgccgcaaccgcattaaatttagatttagcacagtttgaataa

SEQ ID NO: 2 - DNA, 1081 bp, Lactobacillus sake
tacttatcttttggaggagataaaatgtctaacttttttgcattactattggtcatttctttttttgccctatcctattatggcattaagcggtatatgaccaagaaaaagaatcccgaggtttacgccaagatcaagaaacggatctggtatgcacttattatttttgtaatcagttttattggtgtggaggcaactcaacctgctgccgctaaaacaaaagctccacagcatgacaaagtatcaactgtcgataacaaactaacagcggcaaaatctgcggtaaatgcgctttttacaaatagcaagcacactaaattaaaacaagatgtgaccaagcatgatatcactgctgctgacaagcaagtcaaacagctaaaagattcaaaaactaaaaagcaactacaaacatcaatcacgaccgcttcagatttactaaaaaaagctgaaaaagaagctgcattagcaacctcaaaatccgaaagtgctcgcgctgcttctaaatcagaatccaaagctcaaaaagaatcggaagaacgtgctgaaaaagaatcttcagagaaattagccgcctcgatttcaaactcagaaagcgtatcaagtgtccaagctgcttctactagcgctcgtcaagcaagtgaacaagccgcttcaagcgcttgcttctgaaagtattcgcattgctaatgaacggctgctagagctgcaagtgaatcaactgaacgagcagcaacagttgccgcccaaagtacacaagcaacaggcgctgatacaactaccaatcaaggtgtgatcgtcggtaacacaagatctcacatctatcatgtacccggacaagcaacctatcatatgagttctactaatatgattacattccctaacgaacaagctgcgattgttgcaggttatcgcaagtcagaacgttaatcaccagctaggtaaccctttatggttaacaagaataggttcaattcctattagagttgttattgaagaaaaaaataaaaagcacataagaaagagctattaaaccgcggtttaatagctcttttcattctaggatatgatcaatatcacccgt

SEQ ID NO: 3 - DNA, 3534 bp, Lactobacillus sake
gtgactttaaataaacgaaatatcatattcagtttgttgttgttactggtaatgagcattcaaatgatagttactactggggtgagtcttgctgaaagcaacatccaaatagattcagacctagcgttaacaccagaccaagaattatctaatgagaaacaaatcgtgattaaagtaagtgaccaagcaaaagcacagcgtgatttaaagcttaatgtaccggcaggcacaacgtttgatgaaaaggccactgataaattaaatccagagccaacatattcggttgcgttcaatccaaaagataaaacgaatcagttaatacttaaagcgttggtaaaaaagacagttgaaaacgaaccagtcgaaacgccaaccaataccgaaataagtgaaagtcacacagtaaccgaatcaaccgccactagcgggttggcaccagtcgctaagaaggattacgtattagtttttaagattaaagatatcaagacccttgttaatcaacaattaaaactagcgaccgtgctagaaggtaaacgctatgaatctaaaccgttaatttttaaagaattcattacgctagaagatgccgttgctgctcagtctactgaaagtgcaagcaaggcaccagatttagtgaaagatacaatttctctaaaagaaaaacttccagcgtcaccttcaaagtctaactcagttcttgaaacaccatcaatcgatgaccctggtgaactagatggctgggttcaatctgataagaatattatggtaccaattaaagttgatcagtcagcaaatgaagcaaatcgtgaatattttatgtattttggtgggtactacacggggagtacaacgcggccaaatggtagtagtagttggggatcggggcaatacgaggtacctattggcgcctcgaccaaagaaaatgtgcctgatgcagcgttctttcaacttttaagtaaaacccaggctggtaaaatattttccgccgtttatgaccatgggaccaacagtaaccccactgatagtaaatttggtgaaacggggctctctttgttgacgactaacaccgattatgtggatccgaaacagtataatcccagacgaacaaataccaaggtgaatgcgcttggaactgatgcccctaaaaatacttttccaatttataaattaatgtataaaaaagacagtgcccatcctggattacgagcatacgcgatgacgaatggtgctgatggtcaaccggaaggtatgttgcgcgttactttggaaccaatcaatcaaaaaggccggattagagcgcagattcggtacatttctttagataagaaaccacgtttgatggcgggagttactcaatgcatatggatttggatacattacacacaagaacaccgacctactcactaggcggaaccacagggttatatacttatacggataaaacgccatctgataaacaactgtttagactgacttactggaaagatgggtaccccgatgatcctgaatacgaaatggtcaattctcaagatgatagtaaatattatggcaaacaatggggacaatatacaggtggtggggtctctatcctagcaccgggatgggaaatcaatgggacggataagaatattacggatccaggcaaatatcagctttataaaggccatggttacaatcaccccgtttggcactttaaatgggcttatcaggaagtgcaaccaagagagcgatatcaagtgaatatggattttggcctatcgagtgaagtggactacaacaacgttgaagccactaaatcgtatgagaatttgacctctaaagatggctataaccgagttggtgatcgactgaaattcaaactactagggcggaacacggaatgggatactaatttaaccaatatcatgattattgatgctattcctaaggattttatggtggatactgccacctttaagttagtagacagtacgggtaaggaacaagtcttaagcccaagtgtttatgatccccaaacagggcttttaaaggttgacgtatcaggatcaatgggctttaacaaaacagtttatatcacctttgaggtcaatattgctccgtctgcatccggtaagacagtcacaaattcaatggatatttataccgataaagcttccaaacatgattcatccgatgttcaggtcaaagttgaaaaggcgcaagtcttattacacaaacaagttaagaatttgacgacaccagatgagtcctatgcaactgaaacaaaaggaaaattaggggatacccttagttatcagattcatgctgttaatgtttcaactgagcgtgaggttaagccaggcgctgatattgaagatgttttagattcagccttaacaagtgatcgggaagtgatgattgattatttcgatgtcaacgggacaaccattcaacaagaaaccggtcagtttgataataatggacacctaaaattggcatacgctattcctaaagcaggtcatgccacactaacttttacagccaatattactgataacagtcaagaagttatcaataatagcgctcgagtaacctacgatgatggcgaaagtacgcagtctaatattacggttgttaacttaccaaagccgattatcccaattatgcaaaaagatgtgcggaatgtgacaaataacgatgctgaatttgttaaagaaacaacaggtgcaattggtgatgaagttgaatatcgtatcatggccgttaatgcatcaactgaagagcgtattcctaaagagacgatgttcagtgatcaattagatgaggatttagtcaatgatacgccggtgacagttgaatactatagcgtactaggaactaaaacggcgacgcaagaagtctatttccaaaatggtaaattcaaattgaattatgaaattgaacctagtggttatattatggtctacttcaaggcaaaaattagcaagacaactaaatcagttgtgaataatacagcgacattagccgagggaaaatcgaatgtcgctaaggttaatatcatcaaaaagacaccaccaatgcttattaaagacgttaaaaatctaactaaagctgatgaagattacgcgttagaaacagaagctgaagttgatgatcaagttgaatatcggatccaactggtgaatatgacttcaattgatacaaagataggtgctgttttaaaagatgttttagatccggacttgggcgatattcaacaagttaagattgattacctcgataaagatgaaaagattacagccacccaaactgcggatttaactgatgatcaggtgacattggatcacgtgctaccggctggtggccagggaatggcaattgcctatatcaaagcgaaggtcaaagaaactgacaagacagttattaataatcaggccagtttaaccactgaatttggtaaaggtacttctgatcgggctaaattaaacattaaacaaagcaaaggtcgaatcgtggttcgttatcgggaccgaaaagatgaaagccacaagttgg

SEQ ID NO: 4 - DNA, 1260 bp, Lactobacillus sake
atgaaacaagtaccagaaacaaaaatcaaactaacggatgtaaaggagttgattaataaagggtataaccttttaggtgagcttcgtgaagaagcagatgctccagtagcaaaagcagaatttctgacagaagaaattacaaccgtttatggagaagaagcagcgcgtaaattttataatcctgaaaacttcaagcgggaagggtctatgccaaaagtagtattgaaaactttatttggtgaagacggcgtacagacgatagatggaaagaaacaccatcaacgtaaaaattatttcatggatttgatgactccagaacgtatggaagactatagagctattctagatcaaaatcttgcaaccgaactcgatcagcaacatggcacttttgagttattcgacctttctaaaagagttctctttaactctatttgtgaatgggctggaattaatctagcacagtatgatccgaaagaaatcgataagcttgctagtaatcaaatatccatgattagcggagccatcacttctccaacgaaccacttaaaaggagtaaaagaccgtaacgaatcagagggatgggctcaatcattaattaaagaagctcgcaaaaatcctgtaccaggcaaagaaaatcttgccctctatacattcgcacaagcagaagacctagaaggagagttattaccagtagaggttgcagcagttgaattgctaaacatcattcgtcctactgtagcactgacggtttggatggccttaatggggcatgcgcttttcagtaaaacgaacttgtatgaccaattaaaagaagactttgataccctacaagattcctttattcaagaaatgcgtcgctactatccattctttcctatgttaccagcgatcgctcttcgtgatgtagaaattgatggttacgaaataccaaaagatagctgggttgtcttagatatttatggtacaaaccatgacgctcgtacgattgatcatccagaaaaatttgatatcaagagatatattggaaaaacaaaagaaatttcttatgaagaagaatacgaaatgattgcgcaaggtggaggagaatttagaaatatgcaccgttgtgcaggagaatggattactcttcacagtatgcgtgtcttttctgaccaattagtaaataaatataactttagtattcccgaacaagactggaccgttccaatgaaccaatttcctacgtatccaaatagtaaagcattgttgtttaaagaataa

SEQ ID NO: 5 - DNA, 2454 bp, Lactobacillus sake
agtactttgaagataaacacggtgttcgaattagtaatgaaaatacttactatgcagattcaacagctggtgcttcaagtgagcgattcactaattttgcttttgaattcgttaaccgaaactatgaaaccaatgaagacatttatttagttatcaagaacgaagaaacgaaggttgagttagatcgtgttgatttcgtgattgataatccatttgccggtggatttggatttgatatttaggaggtgagatttgtggatcaagttattttagataaactcgtcgcaaatttccctgggaagtttgttcgtaaggatctgactaagaaaattaaggaaggggctaatgttccagtatacgtattagagtatttattgggaatgtatgcctcttcgttggacgaagaattagtcgaaaaaggtgttgaaaatgttaagaatatcttagcgcgtaattatgttcgcccggatgaagcggaaaaagttaaagctaagattcgtgaaaatggtggctacacagttattgacaaagtaactgtttccctaaatgagcacaaggatcaatatgaagctgaattctctaacttagggttagctggcgtgcagattagtgatgaatatcctaaaaaatatgatcgcttattaggcggtggtatttggtgtattgttcaaatggagtactggtttgacgaaaatgatcaaaggaattcgccatttattgctaaaaaagtagatccaattcaaatgccaggattagatattgaggaattaaaaagaggacgtgctgcattcacagaagacgaatggatcgatttagttttgcggtcaattggaatggaaccaacacagtttgatgaacgcgtaaaatggcttcatttagcaaggttggttcccttaattgaaaataattttaacctatgtgaattgggtccgcgtggaacaggaaaatcacatgtttataaagaaatatcacccaattctattttagtttctggtggtcaaactacagtcgctaatcttttttacaatatggcacgtcgatcaatggggctagtgggtatgtgggatactgtggccttcgacgaggttgctggcattaatgtcaaagacggtgatcttgttcaaattatgaaggattatatggcctcgggttcatttgctcgtggtaaagaagaaaagaatgcgtcggcgtccatggtatttgtcgggaatattaaccaaagtgttgatgtgattttaaaaacatccaacctttttgaaccatttcccgaaaagatgggaactgattcagcgttcttagatcggatgcattgctatttaccagggtgggagattcctaaatatcgtccggaattctttacaaatgattatggctttattactgattatttttcggaggtaatgcgagaactaagaaaaacaacctatagtgacgcgattgataagtattttagattaggagcacaattgaatcaacgggatacgattgcagttaagaagatggtttctggcttaattaaattagtttacccagatggaaactttggtaaaatcgaagttgagaaaattctacgattttcaatcgaaatgcgccgtcgagttaaagaacaattgaaaaaaattggtggtatggaattttttgatgtcaatctttcttatattgatttggaagagaatcgtgaagagtatgtgtctgtacctgaacaaggaagtggcacattaattccagaaggaattggaaaaccagggcatatctattctgtttcaaccggggctagcggcagaatcgggacctttacatttgaaacccaaatggtggctggaaatggtaagtttaaagcgactggacttggtggggatggacaagctaaagaaaaagcagattcagcttataagtatctaaatgcgaatgcgagctctataagtgctagcatctcaacaacaacccacgattatgtgtttaatacacaagacttaaacggtgttggaaatccggaaatgctatcgctacctgcattgatagcactttgttcagtaactttaaatcgcccacttgttgctagcactgtcattttaggagacgtaacaattggcggaactttagtcaaagtgacgaatttagctgatatgatgcaagtagcaagagattcaggtgctaaaaagattttagtaccgatgatgaatgctgcggatttatcgaccgtaccgccagaattaatggcaacgtttaacatcattttttatcaaacaccggaagatgcagtttataaggcattaggcgttgaatgagattcaatatatagaaatccagtgttttgcgggggatatttagatggcaattacaactaattataaagaactaaaactatgtaaacatgggttacctacatgggatgcgatgttaccagttgttttattggtagcaggtgagcatgccgagccaatcaaaagaagt

SEQ ID NO: 6 - DNA, 1024 bp, Lactobacillus sake
atggagagaataagtgaatacaaagtattgaataataatgttttagctggagtacaaggtggtaaaaagaaaaagggtggcttcttttggcattattttggagatcctattgttagttttggtaaaggatttattggatattaaataaaattaaggagtattcttatgaataaaaaattggatagtttcagcagtattgaagatgataaattaggactagttattgggggacgaaataatctagcatatggtcttggtaagttagttcgtgccggtgttgatataggaatagcgattgggagcaaaggtcgctacaaaccaagacactaaattaatgaaatcataggtaagataattgaatacgttagaagtgattagccagagaagggctgttggtgggtttgtattattaataataggttatttattgagtagtcgtattagtctagaaatttctttttttaatatatcttgcttatttttaatcttattttcgtatagtgcactgattgtattatttggttatcaatcaacaaaagatttattaaaaccgctaaaacgaccgtttgtgagaaaaatattatcagcagttggattaagttttctaattgtctttggaataagtattgtgttaatgatgttttttggagaagtggcggtcactcagaatggcaatattccattcctgaagtcaattactgtaaagtggcaattagtgagcttgctaattatctttttaaacataataggtgaagaattatgggtcgctgggattgttttaccaatagccacttggctaacagaatataaatttaattggctattggctaatttaattggctgtttaatttttgcattaatccatttagagatttataagttcaatattgcaatatgtctaattgtaggattttctaggtatggattttcgatggcttggaaatcgaatgatacattaagaggtgggatatatgcgcatctattatatgactccttattgttagctgtgaatatactattataa

SEQ ID NO: 7 - DNA, 1041 bp, Lactobacillus sake
atggcaacaaataatttagttttgaagacagctgacatttgtgatattcgtgatattgaagaggcgcgggcaacaatcaaaccttacatccgggaaacaccattaattcaatcgatgtttttaagccggaatgtcgctaagggcaatgtttatctgaaattagaaaacatgcaattaacaggctcgttcaaatttagaggtgccaataacaaaattaatcatttaactgaagaacaacgtcaacgtgggattgtaacagcatcagctggtaaccatgcacaaggtgtcgcattaaccgctaagttattgggaattgatgcaacagttgttatgccagaagaagccccaattgctaaacaagaagcaacggctggttacggtgctaacgttgtcttacacggtgcaacttttaacgatgcacgtttatacatggaacaattagcagaagaaaaaggcatgacaatcgttcatccttacgatgatcgtgaagtcatggctggccaaggcacaattggtcttgaaatcttagatgaaatttggaatgtcgacacagtaattatccctgttggtgggggcggcttaatttcaggtgtggcaacagctttgaaatctttcaacccatcaattcacattatcggggttcaatctgaaaacgtgcatgggatggcggcttcaatcgatgctggcaaaattacaagtcaccatgatgacttcacattagcagatggtaccgatgttgctattccaggagacttaacttacccagttgttcaaaacctagtagacgaattcatcttagtcactgaagacgaaatcgcaatggcaatgaccgacttgatgcaacggactaagattgtcacagaaggtgcgggtgcattaccaacagctgccttattaagtggcaagattgatcctaagtggcttgaagataagaacattgttgcgatggtttcaggcggtaatgttgatttaacacgggtttcaggtattattgaacacctcttcaaaccagcagacacaagcaagggtgttgttggatag

SEQ ID NO: 8 - DNA, 7542 bp, Lactobacillus sake
ttactttttaagtttgtggtgcataatcgtatccactgtttgcttcattcgccaattgtattgctcatcttctaaaagtaacatcgccaaaatatcgataacataggtcagcgcaaattgtgagttcacaaaccgcgcactggcaaagccaatgctatcttcaacaaacaacgtcaagtcactcgcagctgccaatggactatcagtcccactggtaattgcagccgttttagcaccattttcccgcgcattttggaccgcttgattgacctcttccgtattaccagaatttgatagaccaatcactaaatccgttttgttaataatccgagaggtcatcaacatcatatgactctccgtgacgacagtcgcttgaatgcccattcgggttaatcgctgcccaaactccgcgcccgtatagcctgaactacccaaaccatagataaaaagctgctggctggttttgatccaactaaccaactgatcaaggtccgcttgtttaatccgtgcctgtgtcttagtgatgacatcttgatagaattgataaatttcaacggacgtttgtgattcatgatcaacctgtccttgaacccccgtcatcgttggttgggggccctcactaaattgtaatttaaaatcaacgaagttttgacagccaatcttacgaacaaaccgcgaaatggttgaattactaacttgcagttcagcggctagttcatcaatcccaatccgctgaatgctcgcccctttttgaatcaccgctagggccaccttttgttcttgtggcgataaaaaagaatattgttgtttgatatgctcgataattgccattgtaactcctttaaaaaccgagattgtgactttcctactattatattggatatgaagcactttagaaagcacataaaaagacgcccttcttgcaggccgtctttttcgatagattaataggatctctccgttatactaattaaaaattataatatcaaccgattacaatttgtatccaaataccagataaaaaatctagcaatatttttcaattgcagtcgtaatcatcgttgctgattctttagcaatttctaaatcagcttcatttaacggtgctaacggttctctaacagatcctaagtctaattgttcattaattcgtagaatttccttaatcattgcatacatattgccatgacccgaacacatcttatatataatttcatcaatagcgtattgaagatctttagcttcttctaacttcagttcactaatcaatgtatttaattttaagaataatgccggcattgccccatacgtaccaccaattcctgctgcagcccccattaaacgaccacctaagaattgctcatctggtccgttgaaaacgatatgatctttaccaccggcagcacagaacatttgaatgtcctgaacgggcattgatgaattcttaacaccgatgacgttaggattccggcgcatttctgcgtataattctgttgtcaaactaacaccagctagttgaggaatgttgtagataataaaatcagtattcggtgcacctgcactgattgtgttccaatagttggcaattgaatattctggcaatttaaaatagattggcggaattgcggcaatggcatcaacctgcagactttctgcatgcttagcaagttcaacactatcttttgtattgttgcaagcaacgtgtgcaataactgttaatttgcctttggctaccgccatcacattttcaaggacaattttacgatcttcaacgctttgatagatacattctcctgaagagccattaacataaacacctttgacaccctttttaatgtaatattctgttaaagcacgtactctttctggactaatttctcctgcttcatcataacatgcataaaatgctggaataatacctttatacttcgttaaatccttcattataaatccctctttctttttatagttcattaacggcatcaatataagttttagcaatcaattgtggccgtgtgatacttgatcccacaacgacactatgacaacctaattttagagcagtgactgctttttcaggtgaatcgatttttccctctgcaattacgggcgccttggacatcgctaaaacctgttttaaatattcaaaatcattatcggcaatattgcagccttccgtagccggtgtataaccatacattgtcgtaccaataatgtcgaaacctaatcggttggcttctttcacatcatcaagactggccgtatcggccattaataatgtatcaggatactcagtccgcattgtttcgacaatcgttgctaattgttcaccgtgaggtcgtggacgccccgtaacatcgcaagcaacaactgctgcaccagtggcagcaatgccacgcatttctttgattgtaggcgtgatataaatgggtgcatcagaataatctactttatccaacccaattaccggtagctcaaccgtatccataattgcctgaacatctactacggaattagccctgattgcactagcgcctgcttcataagccgctcgtgccattcgtgccatgataaatgagctgtgtaatggttcatccgctaatgcctgacatgatatgattaatcgatctttaaccttatccaaaaaattatttttcaaattcatcaaccttctattctttccctaaataacaacctataaaactttaaaactaattccatctaaattgtattaaccgcttacacataagatacggcagttattaattttcatcaacaattagatatgaaaaaaatttccaatttactaaaataataaaacgacgtagttacattacgccgcttcattattttatcccttataagtttcagagattgtctgaactgttaacccagtgatatctttattgctcttcttgaagaccaagctaactaaataactaattacaaacgctgagataaatgaaacgcaactaacaacaaacggtgaaatggaagccatctgtgcaaaataagttagaacagcactagcaattaagccaatcaaggcacctgttgcattagctctttttgtaaaaatgccaactgcaaagattcctgccaatgggacaccgaaaataccagtaatcgtcatgaataaatcgagcgtttttgattgatctgaaagcatcaaataaatagcgactgctaaactaattaatcccgaaataataataataactctggcccatgtcacatccgaatattgcttgaatttatcattaaagaaacgaactttgaaatcagcaatcgcacatgatgagattgcgtttaaactggaagcaactgtcgactgcgctgctgcaaaaatagcggcaatcactaatccagcgatacctgctggtagtgttttaataataaaataaggcaccaatgctgttgtattaaaacccttaggtaacgattctgcacgactataaaaactatataagacagtacccataccatagaaaattggtactgtaataattgccaatagtccattcgtccatagggatttaacggtttctttcatcgtacttgtcgtttgataccgttgaataacatcttgactacctgtgtattgataaagtgtattaaaaaactgacctgcaaaaattagtggtataaatcgtgagagaacacttgcgttgaaatcaagtccggaaattattttatgatcagccatggcatcattgaaaacagtcccccatccccccttaatgtaatgtgcgcccaacatgctaaccaataatgcgccacctaacaacagaatgccttgaataacatcgctccagataaccccttcgataccaccaaggaatgtataaataatacataacccaccaacaataaccgcaattaaaacaggattaatatcagtaaccgatgtgacagccaggatcggtaagtaaataacaatcgcaactcgaccaatatgatagagcataaatagaatgctcccaacaattcgcatcaatggattaaaacgatgttcgagatattcataagccgttgttacagataattttctaaaaaaaggcacgtagaatttgatcaaaataggaattaaaataataatcgctaaactcccaaccgaataggaccaatcctgtaagaatgcttgttctggtgttgacataaacgtaattgcgcttaacgtcgttgcataaatactaaagccagcggcccatgctggaatctttccttctgctttaaaaaaggcatccgtactcttgctagctttcttcgtaaaataaacaccaacgagtaacattgctagtaagtaaacacataaaacaatccagttaaatgttccgaaaccaattttttgcattttcttcacctctaaaatttattgtgcggtcaatacattttaggatgattggatttgcactaatttttaattaatgctaaactccaactgaaaattaaaaattatgaatggttcaactataaacaccaacttacaaagttgtcaagacttttttgataacgcttacacgaaaggattgttttttatgttaactcaagcttcctatctgacctttgatattggtggcaccaccatcaaatatggcttaattgatgaaaatttatctctcacccatttaggccaattagaaacccagcaaaatcaaaatggcatgataatgcaacaacttatccagactactcggaaagtgatgaccacttataaaatcattggcatcggtgtaagtactgccggtattgtcaatcgtgaattaggtactattatctatgccggaccaactatcccaaactacatcggtactgaaataaaagccattctcagtcatgaatttacgctcccagtctatgttgaaaatgatgttaacgctgctttattaggcgaaaaactagtaggtgctgctcaagatgctaataatatttattgtgtcgcactaggtacagggattggtggcgctcatttattaaacaaccacttacaagatggcggtagtaggtgctgctcaagatgctaataatatttattgtgtcgcactaggtacagggattggtggcgctcatttattaaacaaccacttacaagatggcggtacggctcaagccaactcaattggctatctcttgtatgattctgccacgcaaaccaactatgaacaacgcgcctcaaccttaacactacaagctcaactttcagcacaattacatcttagtgtccctgaagcatttgatgctgctaaacgtggcgaatctttgccattagagattattaaaaattggagccgttcagttgcacaaggtctcgctcaaatcattttgattgttgatccagaactcttgattattggcggtggtgtctcaaaacaggaaaaatttctacttgatctattaaatcaaagtatccctgcctatcttccgcctaatttctatcggacaaaactagtgactgctcataattttaatgatgccgccttgtttggagctgtttatcgttttttcaattaaagcgcttgcaacaaattaaatacgtgttatactttaatcaatttcatcttaatatttaaccgctaaatttaagacgattccctacatagggagtagtctgtcaatttagctttttttatggggtgaaaacatgacaaaaattttaatcaataccatcgtttttcaaaaggaaattcacagtggccaatcacaatatgcactcttaaatcaattaaccgcaaaaatcgctggtttcgaagttcgaggagaattatttaagccagaaacgaaacacgatgaactagtccaacttcaacaacttgctgagcagcgtcattgggaatttcgctattcaatcccagaatgtctttttattaaatctgagattaatcccaatttagaatcacactttaaattagcacaacagtttggaattactgcattaaaaattagtttaggtgaattggaacacataacggctcaacaagttagcttattaaaccaactcattacaacctatcaagttcaactcaatatcgaaaatgaacccaatcaaaatggtattttaaagcatatggcaatgatttgcctagaacttaccaactataactcacttactggctatacctttgattcaggcaattggtattggattaatgaagcaccattaaccgcttggcatcagcttgcaccttacacaactgttttacatctcaaaaatattcaagcacaagaaactgttttactcgataacggtttaacaccatggcggcaactttgtgaatccttatcaccaaacataccaattattcttgaatatccaatgtctaattccgaattagaatcagaactaacattggttgctagtgcattggaggcctaatatggcttatctactaccacttgcattcatcattttattgactaagttgttctctcacctatttgcccggcttaatctaccaactgttattggagaactagcagtcggtgtcctatttggtccagcttgctttaacttgattcaatctaacgattttattacaagtttcgcgcaacttggcgttatctttttaatgtttatggccggaatcgaaggtgatctcagtcagttattaaaatattggaaaccttccctaattgtcgcaacgttaggtgtcattttaccaacattaggtgcctttttcatctgtgtaacactctttcattttacagtcagcaccgctatttttatcggcttaattctaagtgcaacttccgttagcatatcaatccaggtcttcaaagaaatggattatctcaatactgaagaaggaatgatcattcttggcgcagctgttgcggacgatattatctgtgtcattctattagggattttttctagtatcatgcaaccagcatctggtcagtcgctcaatctggttaacctaattttacctaaactattattttttattctagccttcgttttaggtaaatggttcgtcccactaattttaaatctagccaaacgcttaacagcttctgaaaacattactagtatcgccattacactctgtttactgtttgctagcttagcaacttatttaggtatgagcgatgtcttgggtgcctattttgtaggactagctatttcacagactgactataaaaatgttctaacagaaaagattgaaacaattggctactctgtttttatccccgtatttttcgtcagcatcggacttaatattcgtctggcccatcttcaagataatcttagttttatcgtattaacaatcattatcgcgattattggtaaacaattaggtggatggcttgggactcgtatttgtggtctttcctcaagtgctggtcatattgttggtgcagggatggtttcccgtggcgaaatggcattggttgtcgcacaggccgcaattaacgcccatttaatcgacgacagtcattacacagcgatgattattgtcaccgttgttacaactctaattgcaccactattacttaaaaatctttataatgcatcaagcacaaaaaatgtctactaattaa

SEQ ID NO: 9 - DNA, 3162 bp, Lactobacillus sake
gtggaagaacagatattacaatatcgagatgccgtttatgagcaagtcgtcaaggtagggaaatcattagggaatgtgacgcgcttgcggatactagatttattggtgcagggtcccaaaacggttgaaaatttagccaatacgattgggttaagcgttgcgacaacgtcacgcaatttacagattttgaagaaggcgcgcttggttgaaattgaacgacataaaaattttgtaacctaccgcttaacgtcagatcagattaagcagcttatatcgttattagtcacggtcgcagaacagtcacagcctgaattggcagccattcagacaacgttcaagcaacaaactggttcaccacaggcgttaacgatttcggcgttaaaagcgaaattaaccgctgaatcaacgtacttagttgatcttagacccaatgatgagtatgagacgcaacacttgccgggagcgcataatatcccatatgatgagttaccacaacatttagccgaattaccgcaagataaggaaattgttgtctactgtcgcggtcggctatgtgcttatgccaatgtcgctagtcaggcgttgcatgatgcgggtttcaaagtggcgacctttaatcaatcggtgtgggaatggaatcagttgatgagttcctaaagttgacttacaaaaagtaagatgatatattatgagtatcaattcaacggaaacgttgattaacttttgaagaggaaggcgattgtgatgacagcacaaattttaaatgaagccaattttaacgatgagattaaagaaggggtcacactagtcgatttttgggcaacgtggtgtccaccatgtaagatgcaaggaccaattgttgaacagctagcagatgattatgcaggaaaggctaaaattgccaaagttgatgttgatcaaaatcaaaatttggcagcgcaatttggcattcaagccattccaaccttattaatcatgaaagacggtcaattacaagaacaactagttgggttgcaacgtaaagaagcattagcgacaaaactagatgccacattagcataaataaacatttaaaaaggatgacggcgaaggtactttgctgtcatccttttttatttagtagtaagttctaaaattaattcgagcagaaaaaacttaaagcagtctcttttaatgctagtagctttttggggaaggtattggatcagcaataatcctacctaataggactattgctaactaatttataagaatatatattttattataaaaatgtctattataaatataacagagtataatatatataaactaaggagcttagttaagcattttagccaaaaattaatataatggggataattactatgagttcaaataataatatagttaataatatcggagaacaaattaaacaattaaggcaaagtaaaaaaataagtgtagaaaaattatcagaattatcaggactaagttcactaactataacaaagatagagaatggaaaatcgaatccaacaataaatactttatggaagttaacaaaatgtctagaagccccactaatgtctctatttaaaaatagaggaagtaatgtcgttttttcaactgaaaatcatggttattttttagaagatgaaaatcacaagtgggaagtcgaaccaattgtaggtcaatttgatttcgaggtatataaagttcggctaaaagctaatagtaagtattatctagatagccagacttctaatactttagaggtgataactgtactatcagggacattaaatcttagagttgagcatgatagttatattgttcatgaacacaatacaatttacttcataaataataattcgcatgagtatattaatgatactaatgaagatattttgatgaacatagttgtaaaatatgagaacctataatataatatgcaattatatactataatatataattgcatattttggagggttcatatgagagagagtacaagagaagagtgtagaggtaactgtattacttctaggaattatatttgtcgcaacaaatttaagagcaccattaactgcagttggccctttaacaggtttaattaaggacaatattggtatttctggaactattgcgggatttttaacgacaattccgttattggcttttgctattttatcaccagtggcacccaaaatagctgaaaaattgagtctagaatatactattggttattctacaatcgtattagttatagggttatttattagatcaattccccaaacaagcactttattatttggaactattttagtaggtagtgcaattgccatgtgtaacgtattaattccaagtttaataaaaagagaatttaatcatcgcttaggcctaattactggtatttattcaattagtatgaatttatgtggggcaattgcatctggagttagttttccattagctaataatctaaatctaggttggaataattcacttaggatttggcttattttggctttaattgcttgtatcgcatggattccacaattgaaaagacatgataaaccagataagatagaggaattacctatagagaatagtatttggcattcaaaaactgcgtggcaagttactatatttatgggcctacagtcactagtattctacgtgttagtcgcgtggttacctgaaatgcttattcaaaagggattcactgcagaacaagctggttatttattgtcaatgatgcaattatttttattaccatttacatttataattccaattattgctgggagggtcaaaagacagagtaaaattgccataaacacatcaatcctaatgtgtttaggaattagtgggctttttataaataatactattattattattataggaattgcttttattgggatcagtggtggctgtgcatttggactatcaatgatgttttttaacctaaaaacaagaaatgcaagagaaggggcagaactatcaggcatggctcaatcaattggttacttgttagctgcaattggcccaactctctttggctttatgcatgatatgactcataattggaatgtatcattaattattttaattgtgactgctattttattagggggatttggattctttgctggtgaagataattatataaattaa

SEQ ID NO: 10 - DNA, 3046 bp, Lactobacillus sake
gtgcaaaaatttatgaacggctttgtaaagggtgtttatacagtacttttggtaagcatttttgcttttattacgtattactcaatcagtaatttagtcaccggtaattacctatctaattctaaattaatgattgttgctattgtcttttacatattaattatcttggcattggttttattgaccaagataaccagtaaaaaaatattctttatgattgtaacgggaatggcgatattagtcagaattggttggttgattaaggtcccgacagcaccgagttctgattttcaaatgatgcatcatgccgcgatactagcgacgcaaggtgacctctcatttctaaaggaaagttactttcaaagttggccttatcagttaggctttgtttattttcaacattgattatcaaaatatttggtcaaaatgtgcttattctacagataattaacatccttctaaattgcggaattgcatttgttgggtataagattattaatctacactttaaagaaataacagggcgcattgtgtatacgttattgctattttatccagcgtatatttatatgacaggtgttttaacgaatcaatttctagcaacttttttaatttatttagcaatctatctatacttaaagcatgatcaattatgggttaaagcaactgctggtgttttattagcactcgggaatatgatgcgaccattgggcattttgttgattattgcacttgtctgttttgaaattacaaaatggttattggcaccggatcggaaaaatattttaaaatcactcggaagagttaccactagtgttttggcctattttctcatgttgttcttagtgaatagcgctttgcaagtaacgcatttatccgaatatccgctggaaaatagaaatccaacttggaaatttgtattagggttgaatgatgaaaccgttgggagctactctgcaacggacttaagcttaatggatcggtatccacttggcgcaaaacgagataagctgggcaaagagattattaaggaaagaatccaagataaacctaagctcgttaatttaatgttcaataaaagtaagaagatgtggactgctcgggatgatgccctaatgtgggggatggcagataatgttaagttatctttaaagattaaaaattggttgaatgcggttcagtttttgttctatatttttattgtagcaaatgccttattagcagtattcaaatggcgtgaaacatggacggacggttatttattaaaattgatgatcatcggttattttatggtccatttattaattgagattcagtctagatatcgatttttcattgtcccggcatttatcatgttaactgccataggttgggtcactgtctatgaaaagatccactcaaggcaagcaaatgataaaaagactttaaaagaggatatagtaataaggtaggattctaagacgactttattatctatgtttattactcgaaaggacgctttaaaatgacagttcgtaacaatgaggcaataaaaaaatatggtttcttttttctggtttttattttctttggttatctatcaattaactttccgttgactggagatgatttaaactgggggataacgactttaaaaaattattttggatcaggtcagttcttaaattatgatggacgttacgctggtaatagtttaattatcattgctagtcatagtgcggtatttaaggttttaagttacgcaggtattacaacactagttgtctatctggctgcccatttgattaacaacgttaggcaattagaaagaaatacgttattaattcttattttgatgttaacgatgagtacagagttgtttgcgcaagttttagggtggaatgccggcttctttaactatatggcttcattggtatatcccttaattattattaacttagtgaagtatcactattctgattggcaagcatcaaagtatttacgttatagtattttaatagcggttctttcaataatcagttgcttttttgttgaacacgttacactattaaatttagcagttgccacaattttaatgggctatatgctataccaaaaaaagaaaaactacttagtaattgctaatttcattggcacatatattggtggcattttaatgttttccaataaagcttatttaaatatattattacatcatgacagttaccgggaaacaagtttttcattgactaaggtttatcatattatttcacaacaaatggatttttatctcttgattgataatccaattattacagtgttacttgctgtcattctgggttacttaattattaaaaatctccaaaagcgttcgggtaataagttggggactgtggtttcttatctagaattgagtatcctgattgctttcatagaatatcattatattttgtttaatacctttttaaaggacttcggccatcgctatttagtatcatgtttattatcaattttattcttattagtaattgtaattgaaatggctaagttatcactggaaacccacaatatcgaatatattgcattaattgcagctgctattgttttaattgtgccattctttgtagttacaccgtttggtcctcgaggagcgtttgcaagttatttctgtttgtgtttgctagtagttgccttatgcaacgaattgaatgctgttaaatttagcttaccaattatcagaataatgatggtaatggttgttatcttttatgcgggcttagcaacacgaattggtcatgcgagtcgtatcaaaaatgattttataacctatcaggaaaaacataacacgcctaaagattatcgtttatatctagaggtaccgtataatcaatattattgggcggttcatccacaggctactgatagtagttacagagggtattataagattaaaaccatagggggcaagttggtcccatatgctaaatggcgtttaattgaaaagaatacgaatagtaacgcagaaatgttttctcaagttttaaaagttgatagtcaaaataaatag

SEQ ID NO: 11 - DNA, 849 bp, Lactobacillus sake
atgaagaagtggggctttttacttagcggtggttttctagtagcaattattgtaggaatattactgataaatcagcgtccaaaccaagcaagacaacaagcaaatcgtgtccatgacagtgcaccaactttttatttacatgggtatggtggctctggtcgttcgagcgatagtatgattgcggcggcagaagagcgaggtagggctactaaggtattaaccgcaattgtcagtcggacgggacaagttgaattagaggggcattggacaggcaatacaacacgaccgattatccaggtgatttataagaataatcgcaatgccaattaccggcaaaatggtgaatggttcaaacgggtattgattgcggttaaccggcagcatcattttaaacaatttaatgtagtcgctcattcgatgggtaatttaacattagctttctatctggcaaataatgctcagaataaaaaaatgccacaattaactaagttcgtttcgattgcggggcactatgctggtattatcgggatggatgatcgtgcaaatcaaaatcatctggctaaaaatggccgcccacagcagatcaacgccacttaccggcaactaatgggattgagacacagattacccaaaaatcaaattcaatacttaaatatttatggggacttatcagatggtagtgattctgatgggcgtgttagcaatgtctcttcacaatcgttgcgatatttagtggcaccaagagcaaaatcatatcaagaagttcaatttaagggctcgaatgcccaacatagcaaactatatgaaaacgaagctgttaataaagcggtaatcgatttcctatggtaa

SEQ ID NO: 12 - DNA, 5307 bp, Lactobacillus sake
atgaagaagcgaataatctatagtttattaataattttgttattaatgacaactgttttgcagtcactaggtactgtatttgcggcagcaactgccaataatagtagtgaaacaacatctaaaataatagatactagtaaatcgattgcacaagtgagtgagtcttcgcaacttcgaagtagcagccagaatgttgtgcaatcttcgagtacaagtcagaaaaaagaaagtaagaagagctctagttcgaattcaacaggtaagtcggtcatgcaaagtgcatcattgtcggcggctgatgatactggactaacattccatcaaggtaagttagcacttggtggtgggaactacttagcctatacaaatgggacgaactatgatgggattcgaggtaagacaccgactatcactgatcagtcgatttcctatgctaattttgtagcgggtaaagttcaaggggtaccaattactaagaccttgatggaacacgctggacggaataatattggtttatgggcacaaggtgcgaaagattttggacaatacacacgttggatgtcaattcataaagatgatgtaaataaatttgatattgatcttggtgcagatacaccgattgcccccaatcaagctgatttgagttttacaaatttaagctcctcgacaactttactttttggcttagaggctgataatgcgattggaacaagtggaacaataaaagttacggcatcatcattagaagcacaaggaactattttgaagcagactttagaatatacacgtacggtcggtggtgctgcgactacaattactgatgatgtaacgtaccaaccaactgattggaagaccgtctacattaaggagcacatcaccaatacttctggtcgtgatttaaaaggcctcttctttggtcgaacaattgatactgatttacggaatttttggactagtgaccaagggtcaggtgccattggagataagccaaaacttattgctgatggtgatcgcggtatgtacgagaaacaagatatttcagcaacagaaggtccaactaagtatcaggaagggaatgcttctttattttatgattttaatcttttcaatggttctggtcctgatggttggcaaggctttaatctcacaaatgcgcccacaagtatcccatcaacttttattggtaaacgatttgcgacaccacgtgcaaaaggagatcgaacgacgaaggtaaacatcgataccgaactcgacatgctctggtggcctaaagatttaaaacaaggtgaatcgcgggatattggctatgaagttggtttaaatggtgggggtcaaccaagtccaccggaaatcactttgaatcaaaagccgcgagtaatgtatgttgatggtagtcaaaaggatgttgtcttagacgggacagtgacgaacaaaaacccaactgattcagctgatcctaaatacaagaagatgaatgaattaatctattatgaagtgcaacataaagatgctagtggcaatgtcacgaccggtggccctaaagttctgacatcagtcgcagatgttgcggctggacaaaccaagaattattctggtctagctaaagggattgaagatgacctacacgtcggtgatcggattgttgtctgggctgtcgatagtggagggatgacttccgttaaccatgaacacactgacttagtagaacgtgcggctgagattagcaaaaaggttaagaatatcacgaatggagattcaagtttcaagaccgaaaccgactataaaccgggtgatacggttgaataccaagggacgttgaagaatcttaaaacgaaagtagccttacctaagggtgcaattattactgatgaattagactcaaacttgaccgtcaaaggtgatgtaacgttaacttactatgatgcgaatgacacgcagattggcgcagctcaaaccacttcgtttgatagtaatacgcatactgtaaaaactgctaatgaaattccagtcggcggttacgtcgtgattgacttcaaggctaatgttgctaagaccgcaacaggtgatattgataatatcatgcggtctaaggttactgttactgaaaaagatacacaaggtaatgaccaaacatatgaagttaatacagatccatcgaataaagcgataatccatccaggtgaagaccgtgaagttcaagatttaaaacagtacattaaacttgataccaacactgattggacattagggacaaccggggttactgggcatcgtaaggataaagttaactacaaatttgcatttactgctaaaaagagcaattcggctggaattagcgatgctgaaatcagcgacattaagatggcgcctgatgaattaacagcaccaacgaatgtaaaagttaagattataacgccttctacgactaaagccgatacatcagaaacagttgatgggacagctacaccaagtagtgatggtgagacttattccattaagattgataagtcaatcaaagctggtcaaaaggttgaagtaacattcgatcgaacagttaatgatgatgcaacaatcgatacgacaacaacagaacatgatcaaacagggaagttaacagcagcttcgttaagtacaccgattgccgataaggataactttaaccttgcaaaattaaagattgaagaccaaccaataacggtaactgatctaaaacaaacgattagtaatacaaccacaccagcaaatgataagaatgatgcaacaacgcatgttgctgttgaaacgtcaggtaaacaaaatgacatcattcaatatacgtttactggtaaagctggggataacacagacgctataaaggatcttctgctttcttcttttaacatgaataaaccaaacgaaatgtcttatattgatgattcattggaaattacgattggctcagaggtacaggaaaaaacaccgcaggttccaggtaatggtaacaccgttaagattacaagcgacttgcctaagaatacgacttttaaagtaacctatcagatgaaggttgtaaatgatgtcgcgaatacaattaccaacgatgcaaagttatctgccacaaacttaaaagcaacaccatttaatacaacgaccttgaatacagccgcggcttcaaatacggcgacaatcaagcaattcattaagaaccggaatacggctggtgaaacttggaaaggtcctaatgtgactggtgataaagctgaaacaagtggtgttccaggaaacattatcgattacaaatttgctattgcaccaggtgcgaagaatagcgctgatttactagatactgctttgaaagatattgcgatgaaagaatcgagtgggatgactttagttaacccagaaggttctatggataatacaaagcaggtgaaagtcacagttgatggtgtgacaccacaatatattagtggtaatcccatcagtgcgaatgcagaattaaacgatgtcttcacgccattaacaaaaggcaaagggatgacaattgagtacagcgctaagatcaatgataacgctggtacgcaaaatgtgacgaacgatgctaacttctacgcatcaaacttaacgggtgatatgccagcaacctcaacagtagctgacaaggcacataaaacaccagctaacaaatcaattttacacattgtgcgtaaagataacgtcacaatcaaacaagaattgaaacaagattcggcaaccacctttaagacgaccgaaactggtgttaaaggcgatacaatcgactatcgtttcatggttactgctggcgatgataatagtaccgatattaagaatattgtcatcgataacattgtgatggatccagccggcaagttggattaccaagctggcgtcacagcaacgcttcaggggacagcgctccctgctggggatgtcgtcatgagtgatggcacaacagccggcacgaagaagattacggttaagaatgtttcattgtcgaaaacacaattattaattgtgaactacaagatgaagattacagcggatatggatggcgacgtaaacaacgatgggctgttaacagctgatagtttcacggatcagaccaacaccgttgccgctgataaagggaaatttaatacaactgttttaacccttaaaaagatatccaataaggcaaagattagccaaagtattaatcttaaagataatgtagcgaatacaaattcaggatggattgggccggatgaaccggcgcctaataataaacctgcggaagcaactgttaaccctggggatacagttacgtacaagttttcgattaaatttccaacgaccgatccgaagaataacgctgatctattgaatagtgcgttacaagatattgcgatgaatgtaccggatgatttagagctagcgaatgttgtaggtacgactcataagattcaaattagctatcggacgggtactgaaggtcaagtttctgagtataacgatactattaattcgattaaggacattaaaaaccttgaccttaagagtccactttctaagaatgtgacagaggctcttgtttactacggcgctaacatcatgccggatgctaaaacacaagacgttaccaacgctgcgaacttctacgcatcaaacttaacgggtgatttaaaagatacgactgttgctaattatcaaaacaagacaaaagcgaaccaatcaattttacacattgtccgtaaagataaggtgacaatcagtcaacaattgaaacaagataaagcagcagaaacagcttttggtccaaaagcatctggtactaaaggcgacacaattgattatcgtttcaaggttactgctgataaagacaacaatgctgatgttaaggatatcgtcatcgataacattgtgatggatccagccggcaagttggactacctaacaggcattaccgcaacagcgggtggaacagccgtccctactgctaatgtcactatgagtgatggcacaacggcgggcacgaagaagattacgcttaagaatgtcgcgttgaagaaatcacaagtattaatggtgaactacaagatgaaggttacagcggatatggatggcgacgtaaacaacgatgggcttttgacagctaacgggtttacagatcagaccaacaccgctgccgctgataaaggcaagtttaatacaaccacattgacattgaagaaacaaaagaacacggcaaccattcaacaatttattagaaatccagcaacggctatttcgggacaagcgtggtttggtcctggatatgggggatggtaa

SEQ ID NO: 13 - DNA, 3398 bp, Lactobacillus sake
atgacgactgaagtaaatgctgttgaagcgcctgctaatgcgtcttcggaaacgatcactaaaatagctcaggacgctatttcaggttcaacaccaactaaacaatcaaatagtgtcattcaaaaaaaggcggcttcagatgcggaaacacaaaccataacgccagcaacgtcaggtgaaaaagcgacgctacttgaagcaccgcagactaaaaaactagccaaccccaaaagcagtaacttgatcaacgagcaggcaccagtttgggcacagtcacctggtatatcgatgatacaacggcgaccttacatttatcaggcggtgtattgccggataaagtggctaatgatactactaatacaccttggtatttccctaactcgccttatattgcgcgaatcgcgcatatcagcattgatggtgaaattacagctaaagatgtctcctatatgttttgggggttgaccaacttaacaacggttcaggggttagctaatctaaaaggggccactaattttacgatgttatttgccagtgacagtgcgctgcaatcagttgacgcgactaacttagatttttcaaaagtgacagcaatgaattcgatgtttagtgattgtgccaacctagtatctgttggcgacacagcgaattggcagcttgggcaagtcaccacaatggtaagatgcttcagtggtgataaaaaattaagtcagttaaatagtacgaattgggacacttctaatattcaaaatatgaataatacatttttcaattgtacggccttaacgaacttagatgtttcaaaatggcagacggcaaaaatgactaatctggggagtaccttttcacaatcgggcattactgttttagatgtttcaaattgggataccagtcacgttactaatttaagtagtacttttctgaatactagtattgccgaattagatgtttcgaattgggatacaagccaagtgacgacaatggcttatactttttcgggatgttcaagacttgaaaccttagatgtctcaaaatggcagctcggcaaaaatacatcgttgtcttacacgttcagcggtgatcaaagattaacgcagttagacgtttctaagtggcaaaccgctaatgtgacgaatatggcttcaacattttccgggactgtcggtgttaagactttagcggttaaagattggcagaccactaaagtgataaccatggcgggcatgtttgccaagagtggcgttgatcagttagaaattgctgattgggatacgagcaacgtccaaagtatgcgactcatgtttgatgcgacgaagttaacaacgcttgattatcctgattggaataccgctagtgtaaccgatatgagttatatgctgcgtgggatgacgaaattaacggatgcttatttcacgaattgggatacgagtcaggtaacgaatatgggcggcatgtttttgaatgatcaacaattagcgcacctgcggttgggatccaaatttaaattcttaacgagtacctcaactggaccatcattagcagaacccagcactgaaacaccctattttgggaagtggcaacgacatgatgcaaatgataatcaagttgggaatacctatacatcagcagcattaatggcccagtacgatgggacgactgttccaactggtgattattattgggccgtcgcaacacccccaacgattaccaagttggtacgtaatgtgacagctgatggtaataatgccccatttaagacggcaacaacggctcaaaagggtgacacagttgattaccaagtcaatatcacccagccaagtggtcagcaattagatcggggtgcggtctttgaagatgtacttgattcacatttgaaatttaataataataaggaactatcaatttcatattctgatgaaaatggtgattttcatggagaacagctcattaaatttaacgatcaatggcagattgcactcggcgaaaatttagcaatcggacaaaaagcacaagtcctaattaaggcgattgtcaacgacgatagtgcgccagaaatcgataatcttttcaaattggtttctggaagttacggtgccggtaccacttcaaacactgctatcgtacacgtcaagaaaccattgacgttaaccgaagcgataaaaaatgaaacgacggatacggattgggacaccaaacaggatgtgagtccgggtgatcgagtcggctttaagctcgattatcaaaacacgacgggcgccactagcaaccagataacgttcaatgatccgttggtcactaatgaactgacgtatcaaacgggttcactcaaagtgacctatcaagatgggacgactgaaactgtcagtgatgcggcgcaaacgcaatttgcaacgaccggaaaattaacgttaaccaaatcgttagcgaattatgaagccgttaagctcagtttttcagcaacggttaatccgttggttacctcaggaacgaccttacacaataaggcaacattgaccgcagataacgtgagtcaaccagttacctcgaatacagttgatatgaacgtggtccaatcagagcatcaattaaccattcggtatgttgatttagatgaggatcttagccagcctgctagtgcggggactcaaattgccgcgccgattacggtgtctggtaaaactggcacagctttatcaacccttttaccagggcagcaagtcgcccctaaggtgattgagggttacacgatttattcagtcagtgaagatccagatttaaaaccggagaattggcaaaaagcgtatcgcgatgatcctcaaattggcgaccaagatcgaatcattacttacggttataaaaaggcaatgttatcgattgatgcgcctagcagttgggaatttggtgattataataataaacccatggaccggacgtattatttaaatcataatcagggcacgccacaagcagtgacggtcaccgataattatggcgttcaaaattggcaattacaggttagccaagcaaaaccgtttgttgatagtgaacaacatgagttaaccgatgcaaaatgggccttttcaaatggcaacgttaagacgttatcgaacaccgatgtggggactgtcactaataatagtgagcacttcacattggcatcaggccagagtgccactttaatgacgatgactaagagtggtcatttccaatccgattcaccggataccagtgatcccgaaaatccctatacccaagttggacaagggcagtgggcttatcgctttggtgacgaaaaaagtgccgattatagtattggtttaaatgtaccggcaactactaaacgatataccggtcattatcgaacgaaattgacgtggtcactatcagttggcccatag

SEQ ID NO: 14 - DNA, 1327 bp, Lactobacillus sake
taacgttgggatcttagcgtttaattgaccaagaccaccgttcaatgtttcagaacctgttgccaatcgactaacgccacttgttaattgtgggacttggctattcaattgaccagtaccagttgctaattgttttgaaccagcagctaattgaccaacaccgcttgtcaattgtggcacttgaccatttaattgtgttgtaccagcatacaaggcttttgaacctgttgctaattgaccaacaccgcttgtcaattgtggcacttgaccgtttagttgtgctgtaccagcatataaagcttttgaacctgttgctaattgaccaataccacctgtcaattgtgggacttgaccgtttaattggctagtaccagcatataaagcttttgagcctgttgctaattgactaataccacctgttaaggctgggatttgaccatttaattggtttgttccggcagctaattgtgtaccacctgcaactaattcaccgttcttacttactaatgcttcagaaccattagcagcagttgcgacacctgttgtatatttacctaaaccagcgatcactgcttgtgaccctgtttctaattgactaataccagcatctaacttagctgcaccagatgctaaaccaggattgccctgtgcatcatcgttcaagccagtttgaattgctttaagactacctaacattgaaacagaacctttaacagctgctgttaactgtgcgtattgtgcagctgttggtttcttagcatcaattttagcattgataccagaaacaatttgttgtgcttcatccttagctgaagcggagagttgtgcactcaacttagctttaatgcctgcttcttgttgtgcactgaagacaatacctgcttgtttttcagcatcaataattgcttgtgcttttgcattgaaatcagttgttgcaacaacttgatcaggaatcgcatttaaatctgtttgtaatgcaggaatcgtactttcaacttgagcaatcataccatttaatgctgtatattgcgcaacggctgcattataacttgcatcatctttgaatgttgggttgctgttaagttgtgattgcatcttttgtaacccagcagaaatcttttgactacctgcttttaagtctttaacaccagatgctagacttgttgcaccgttatttaaatctttgttgtttttagcaagttcttgtaaaccatcatttagactacttgtgccatcagtataagctgttacacccttacttaagctagcagcaccattatttaaatcttttgcacctgaggctaaatcgcctgttttaccatttaat

SEQ ID NO: 15 - DNA, 500 bp, Lactobacillus sake
ttaataacaataaattttaaagtatagtcaaggaggcgattagaatgttacaaccaacaattcagctatcggcacaagcttatgtgatccaatggaatcggtttttagagcggtttattgtcttcgaaacggtttttagtgaatatgcgcaaactagtaaaaatgttatgcggggtaaaggattggtagatgaaaatttacaattacttaatcactattccagacagaatttaactgtcctcgatcaatttgttgatcaaattgcgcgtattcaaccgccaacggaatttgtcgcattacatgagcaattacagatggttattaagcattatgtgactgctgcaaaggattttgtcaatgcgatgcgattgactgaagagtcggaagtggaacaacaattaaatcaatgtcgaacgattcaaaaacagcaacgactaaaattaaatcaactcttaaatagcattggtgcactaaacagttagtggcgtcatcaaaaagga

SEQ ID NO: 16 - DNA, 141 bp, Lactobacillus sake
attgaataccacgttggtcaaccggaaccacaaggaaataatgcgagcttaccaggtggtggtattatacttaaacagtattacggtgcacaaggaataaagatttttaaactaacgcctaaaccaggaaacaaggtcaat

SEQ ID NO: 17 - DNA, 1476 bp, Lactobacillus sake
atgaaggttaatgagttatttaaaaagccaatagatcgtgagattcaaggagtcgtaaaaattggccaagatcaagaatcaaatacatatcaggaattagatgaatatgtgattactagcgaaatacgtaagaatatgggaactttttttgaggcgtacgtaaagtcgttagagcaaccaactgatcaaatgggtgtctggatatctggattctttggttctggtaaatctcatttcttaaagatattaagctacctaattgatagtcaaaagacagttaaaggacgacgacctatatcgtttatcgaagataagtttgaagaaaatgatgaaacattagcgctaataaaacgcgctagtgaacaacctaatcaggttgcattatttaatattgaatctaaggctgaggctgacagtatgaatagtaaatcggctgttgttaaagtctttaataaagttttgaatgaattacgaggcttctcaggtgctaatgcatggattgcggagatggaagagacacttgccaataacggacagtatgaacagtttaaaactgcttttgagcgtattgctgacttagattgggtggaaggccgggaggaaattttttataatgaggactcggtaattgaggccttaagtgaagtaacagaaatgacgattgagagcgcacgacacttcatagaatctggggaagctaattatgaaattagtgatgagtcatttgctaaaaaagtgaagcgatacgttgatcaacagccagataattatcatttagtttttttagcagatgagatggggcaatatattgctgataatggtcaattaatgttggatttacagatggttgttgaagatctagcgcaatattgtcaagggaaggtctgggtagttgtaacaagtcagcaggatattgattcactaaatcgtggtggtatgtcctctaatgatttttctaagattcaggggcgttttaacacgcgattgccattgagttctgctaatgcggatgaagttattaagaagcgattattagcgaagacagaagaagccagcgcgatattaacaactgaatatacagaaaacgaagctgttctaaaaaataaaattttatttactaatgatacggcagaaatgaaatcttttgataatgtagatgaatttattgatacttatccttttgtgccttatcagtttaatctattacaaaaagtatttatgggaattcgaggacatggtttcgctgggaaacatttggcaaatggtgagcgtagtctgctatctgcagcacagtatgcggcacaaatgtttagtgaacaatcgaccgattttttggtaccattctcggtattctatgaaaatattgaaaatgcactagaatattctgtgcaaacacctattattcaggctaaaagtaataaacggttggataaatttgatgtcgaagttctgaagttattgttcttaatacgctat

SEQ ID NO: 18 - DNA, 866 bp, Lactobacillus sake
atgaaagcaatcgttgtctcaaaggctggtgggccagaagtccttacatataccgacgttccaaaaccaacaattaagtcaggctggtcactcgttaaagtgatgggttttggcattaaccgctccgaaattttcacgcgggaaggcaagtctccctcggttcaattccccagaatcctcggcattgaggctgttgggatgattgctgaatcttccgatccggaacaactgccagttggtcaaaaagtgatttcaattatgggcgagatgggccgcgcctttgacggtagctatgctgaatacgtcttactacccaatgaacaaatctatccggttgaatcgactttaagctgggctaatctcgccgcgattcccgaaaccttctatacagccttcggaattttcaaaagtttacaaattactaaaagcgaccatgttttagtcagagcagccactagcggtgtcggtattgcagtgatgaaattaatcaaaggatatgctgccagtatctccgtaactgggaccacgcgctctgcaaataaatctgatcaattattagcagccggctttgatgatgtactcgtcacacccgcacccaacaccctcccaagtgagtccggccaattcgataaaatcgctgaccttattggacccgcagctactcgtgactcattacgacatctaaatgagtatgggattttaagtgccaccggcgaacttggtggcgtctggacgattgatgccttagaccctattatcgatattccgaataatcgttacctaactggcttttattccggcgatgtttctagcagcaaaattcaggaaatgttagcctttatcgagaaatatcatattgatgtgac

SEQ ID NO: 19 - DNA, 1587 bp, Lactobacillus sake
atgtgcggatttttagctgttgattctaaggaatttgatttaacgaccttttgcgacgccctggagaaaaacgttgaccgcggcccggacatgaccgaaaccgttgaagaagacgcggtgatgtttggtttcaaccgcctggcgatcatggacctgtccgatgacgggatgcaacccttcaagggcgaagattgcaccctggtctgcaacggggaaatctataacttcttacagctcaaggagaacgtaaaagacgacttcagcttccaaagctctagtgactgcgaagttttgatcccgctctaccgtaagtacgggctggacaccatgtgcaagatgctcgacgccgagttcgccttcgtcttatacgacaaggtggccaagaaggtcgtggccggacgtgacccgatcgggatccggccaatgttttacggctacaccaaggagaagggcgaaatcgcctttggttccacggccaagaccctgatggacttgtgcgaccaaatcttcccgttcccaccgggccactactacgacggcgaaaagttcgttacctaccgcgacccggccatggttacccggatgcacacgccgagctttgaagaagccacgaccggcatccgtgattacctggtcaagggggtggaaaagcggctccacgctgacgctccggtgggctacctgttgtccgggggcctggattcttccctggtttgctcgatcgcatctaagttaatgccgggcaagaagttacggacctttgcgatcgggatggaccgcaacccgatcgatctcaagtacgcccgcgaagttgccgactacttaggcactgaccacaccgaattcatcatgactcgcgacgatgttttaggggcgctgcgtaaggttatttataccctggaaacctgggacattacgaccatccgggcctcgatcggaatgtacctattgtgcaagaagatccacgaaacgaccgacctgaaggtgatcctgaccggggaatgttccgacgaaatttttggctacaggtacaccgactacgcgccaaacgccgaggccttccaaagcgaatcgatgaagcgggttcgtgaactgtacatgtacgacgtgctgcgggccgaccgttgcatctcggctaactcccttgaaggccgggtgccgtttgccgacctcgacttcgtggaatacgtgatgtccttagacccggacatgaagatgaaccactacgacaagggtaagtacctgttgcggatggcctttgccggacaagactacttgccagacgacatcttgatgcgggaaaaggcggccttctccgatgccgttggtcactccctggtcgacgacttaaaggaatacgccgacgccaagtacacggatgaagacgtggccaaggccagcgaaaagtacgcctacaagacgccgtttaccaaggaatccttgctctaccgcgacatctttgaagaattcttcccgggcaaggccgaatggatcaaggactactggatgccaaacagcgactgggaaggttgcgacgttgacgacccatccgctcgcgttctctccaactacggggactcgggcaag

SEQ ID NO: 20 - DNA, 2462 bp, Lactobacillus sake
acggcatggtctatgtcaaccgcacgaatcacgatttgaaggacatgcaacgtgtgccaaagaagagtttttattggtttaaagacttgattgctaagtatcagtgttaaatgttttttaaaaggagtggggttttcgccactcttttttatttacctatttcatttcaaacttattcttgttataaccgataccagttgtccgtgttataatacagcggactaaatgttataacagaggagagtacagatgagtattggcgatattttaacgaagacgtttacgaacatggatattgtgagttcgatcacgtcgacagtgtttattattttattaggattctactgccgcaaacgcgagatcttcacggataaatttgggaagattctgtcaaaagtcgttttaacggttgctttaccagctttggcattcaactcattcatgcaagatattaatgggaagactttaaaagaaggtatcaacgttttagtttggggcttcttgatctatattattttaattttaatttcaaaaccattatacttacgctttaagggtgatcaacaagatacattgcgcgtgttgaccatctttggttcaacaacgttcttcgggattccaattgttggagcagttttcggagctaaaggggttatgtatagctcaattttcaacattggttatcgcgtgttcttatactcatatgggtacatcaagatgagtggcttgaagatggaatcgaagaacttgaaaaacatgttcttaaatccaatcgtgatcgccacatttgcggggttaatcatctggttaatccaaggcgcattaccacaagtggcggttgctgggcattcagtggccttcttacgaattgatcaaacagcgccatggttgttcaaaccaatgacgtacttagctggtttatgttcaccattagcttggttagcaattgggacaacgcttggtgaaatcagtttcaaagaagctgcttcggacaagacatcgtggatttatagtattaacaaagtgattttagtacctgctttaaacatcgtgttattggccatcttaacaatgacccacattttaccagttagctatatcagtttggcaacgattgtcatcatgatggcaacgccaacggcaacagtcgccgctgcttacgcgattagttttgacaaggatgcggtcttagcatcgaacgcgtcattactatcaacggtcttggctgttgtgatgacgccagtctggttagtaattttagaagtaatcaaccaaatcggtattttctaggaagagggaaattaaatgtttaaaattgcttgttacggcgtacgccccaatgaagtggcgacgtttaatgaatataataactatgattatgatttaaccttaattgaagaactattaacacacgctaatatcgaaacagctaaaggccatgacgcggtattgttacgcgctaactgtgccgctgatgcgcaaaatatggcgaagttgaatgaatatggcatccattatgtctttacgcggacagtcggttacgggcatatcgatttagccgcagcaggcaagtacaacatggaagttgctcgcgtaccagcctactcaccaaacgcgattgctgaattagctttgacgttagggatgtcattattacgccacaccacttacacgacgaatcggacagcacatcatgattttcgtgtgagcccagtgatgtttagtaaagaagtccgcaactgtacggttgggattatcggtactggtaagattggctataccgaagcaaagctatacaaaggcttaggtgccaaggtagtggggtatgacttgtacccatccgaaattgctaaggaagtccttgattttgttagcttagatgaattattggcgcaaagtgatattgtcagcttgcatattccatactttgctggtcaaaacgatcaatttatcaatgctgatttaatcgcgaagatgaagaatgatgccatcttggtcaatacagctcggggtcaattgcaagataaccgtgcgattatcgatgctttgaaagctgatcaacttggcgcatttgccgctgacgtattaccagctgaaggcgaaatcttcttcaaacaattcccagcagaccaaccagtccctgatgcgacaacccaagaattaattgatttatacccacgcgtcttgttgacaccacacgttggttcgaatactgacgaagccctcaagaacatggtcgaaattagctttgaaaacttccatgaaatcttgacgacaggtaagacgcaaaatagcgtcgcaaccactgaaaattaaagcggttatttgtattttcatgaaaattatggtatattgttatcagtaattagatacttcagagcgagtgagcagaaatgctcactccttttattggagcacgtctaatctgaatatataaatgaattggagggaatcatttgagt

SEQ ID NO: 21 - DNA, 576 bp, Lactobacillus sake
atggaaaagtttattgaattatctttaaaagaagtaacagcaattacaggtggaaaatattatggtaacggtgtacactgtggaaaacattcatgtaccgtagactggggaacagctattggaaatatcggaaataatgcagctgcaaactgggccacaggcggaaacgctggctggaataaataataagcaaaacttaatttagatttaaggactctctatcatttgatggggattttttagcttatagtaacagaataaaataagaggtgtcagattatgaaaatattgaagtggtattcaggtggtaaggatcgcggagaacgggcaaatgatattataggccaattgttgctagatctgaaccatgatccaaaaaatgaacatttagaagcaatattaataaattatcagaatgaaattaaaaggaaagaaagttcggtgccatttattttgagtcggatgaatatatcaatagccaatacaatcagaagagataggctcattttaaccgattttcaagaagataaattgaaattgttaactgcgttgtctaatataagatatggctattag

SEQ ID NO: 22 - DNA, 336 bp, Lactobacillus sake
atgaagagattatgcatattagcttatcaagggctgccatttctgattgataatttggttatctttaataagaaggaacggaattttgtgaagaagttaaacaatagaatcgatgaactatcgatgagttggcaagtagaacttgatagctcatttggaaatattagtgatattgaagtcaaagcaccgcaagccgtgttattaaaaaatggcttgcgatatcgttttaatactggcagcttccctaaaaatgatatctatcaattgggagctttagaattacaagaaggtgatattgatgccgttatttcttttttgaagcatttagagaggtag

SEQ ID NO: 23 - DNA, 482 bp, Lactobacillus sake
taaaatgcgtaaaataccctcggtggcagttgtgtcgctgttcgggtcattggaaaaagcgggtgatgctacacttagcacgtagtcgatgttctgcatggctgtcgcccagccagcatcttgagtgagatcagcggcgacaaaacttaaattttcgcggtgttgcgcctgattttcttttagggcggtgcggacggtggtttgtttgtcgagttggcgtaaggtggcacggacgtgataaccttgatttaaaagttgtaagataatgtgcagacccagataaccgttgccgtcggtaactaatacgttttttgtcatgatgttatgctcctattccctaatgagggttaatttaatcagtcatgtatgttatgccgttaagtagcatgatccttgtttatggcttctttttatttataatagtccctagagtatactctagggcaagcaaaagaacactatttcaacgcagcgatttttac

SEQ ID NO: 24 - DNA, 3211 bp, Lactobacillus sake
tatttaagaatattatgttgcatatgatctctcccttaattttagttcgactaaagtgtattagcttttgggagatataaacttgacgtccatcaagtttataaaatagttctagatacttaaaatgctcgtattgactaatataataatggaatatacaatagaaggcgagatattgtgaattattaaactaaaaggggtggcacggaatggaaaaccaagaaaaaatggcggcttcaatgttgcatagaatggtccgtgaatttaccgaaaatgaactagcaccgctggatatggaaatcgacgcccagggtgattatccgcaaggcttattccaaaaagtgatcgataatggcttgttaacaatgacactaccacgtgaatatggcggagcaggcattagctatgacagcgttgccaaggcgattcacctgatggccattggtaatgccagcatggcagttacactggaaggccactttaagacgttggaacagctcttaaagtatggtgatcagcgcttaaaggatgagtatctgacgagtgcggcccatcgaatttttgccttctcgcagactgaatcgtctgggggctcaaacccgcgtgggattgctacgcgggctgtgaagagtggcgatgattggatcattaccggcgataaaattatgatcacgaacggcggcctcgcccaggtatactgtgtgttggcaaaaacggaagatgatcaaatggccgtgtttgtcgtggatcaggatatgcctggtttttcatttggaaagcgtgaagacttcattggtctacgcgggacacccgtcggcgaaattgtgatggatcatatcaaggtgccgggttaccacctacttggtcagatcggccaaggcctcgaaatcggcaataacgcccacgacgacgcgcggatcttgatgggcgccgtcctcacgggcattatggaacacgaattacagattgcggtcgactattcgaagcagcgtaaagcactcgataccccgctttatgaattacaaaccattcaagaaaaaatttcagaaatcgcgattgcccgtcagaacacggagctactgtatcagcatgcggcacagttgaagatcacgggacagtcgtatgccaaagttgccgcgatggcaaaatcgtacggaagtcgttcggcggtccggtgcggcgatatggcgcttcaagtactggccggctacgggtatagtcgcgagtatccgatcgagcacctcattcgtgatgcgcggaccatggaaattgctgaaggtacggtagaacggatgagcggtgaaattgcactaaaacaggttaatcagtaaggggtgaacaacatgaaaattgtggtctgcattaaacaggtaccaagaagtgattcggtcaaaatcgatcccaagacgaacaatatggtgcgggataatgtcgaaggggtcatcaatccatttgataaaaatgcgatcgaagaagcactccggattaaagatcaacttggtggagaagtcattttgctgagcatggggccggacggattcatgagttcgctgcgcgacggtctcgcaatgggggctgatcaggccattctcctcagctcacgggcctttgggggcgccgataccctcgcaaccggctatctaatcagtcaggcaattaaaaaaattggtggcgtggatttggtattgttgggacgccaagcagtcgatgcggataccggccaagtcggaccgatcgtggccgaatacttaaacgtgccccaggtcacctttgctgaagaactgcggtttgtcgatgaacacaccttgtacggccaacgcttattggatggcgcaacgcaaagtgtccaagtgacattgccagccgttgtaacagtgcgcagtgaattaaacaccccacgctacgaaacaccggttaacattcaaaccagttttgaaaaaccaatcgacgtgtggacggaacacgatctcgacttagatgcaacacgcattggtcaggccggttcgccaaccacagtacaaaaggtgtacgcaccaaaacgtgagagccgtcaaacggttcaactcagtgatgacagtgcaacggcagtcaaagaactgctggcgcggattcgaaattagcggggtgaataaaatggcacaatcaatgattgcagtttatgtgcaaacggacggttcgatggtggagccgaccagtttacaactgattaccaaggccagagcaattgcgaacggtcagcgtgtggtggctctattgccgaccagcgacgcggaccaaaccgttgcgacggtcaagcagtacggtcccgatgaaatttttgtgttagccgacgaccggtttgcggcagcgacggacaccgagattgcggatggcctctatcaggtcgtgaaacaggttcaacctaacagcatgctgattccagcaacggtggttgggcggtcggtggccccacggttacaggcaaaactccaaacagggttaacggcggactgtttagacgtctactttgatggtgaaacactggttcaagtgaagccaacttatggtgatgacatcatgtgtgaaatcatttgtgcggatcgccggccacaaatggcaacggtccggcccaatgtatttcaggctgaaccagcgcaagttgctacgcaagtaacgaacgtggactttgaatttcatccagaagcacacattaagatggaaaaaccgacaccaattatcagtagttcggccaacattagtgatgccggtgtggtgattgccttaggacggggggctgataacccgcaactgatcgaaaaagcgcagaaattagcggctcgtctgggcggaatggtcggtgtttctcggccattaaccgatcacgcagaatttggacatgaaagccaaattggacaaagcggccagtctattgcacccgatttattgattaacttcggtatctcgggggccacccagtacctagtgggaatcgaaaatgcgaagacgattgtttcggtaaataccgatccggatgcgcccatttttaccaaatcagattatgcgtacgttggggattccaatgatttcatggataccctgttaagtagcacaagagtttaaaaatcaacgccagcgactcttagatagggatcgttggcgttttgttgtgcgaagaactttacaccctgtcagtgtgctcttatactaaaaaaatggactatttttattcagt

SEQ ID NO: 25 - DNA, 390 bp, Lactobacillus sake
attgtaaaattacgtttcttacaccagctaaacagttcaacagctataatggaaccgaacattgatacgaataaccctttagtcccaaaatacatcgtatcaaccataccatctttagtgaaatcaactgctagaattaaaaatcccattactgccattgtgacacacccaggaacatccaatttcttagactgagctagatgatacccaataattgcagcgacatataacgccatcatccccatcccgacagtatacgggatactgataacactagaatattgggcaatccattgagacacaggattattcttaccaatcatattaggaatagcatctggaattaatgcaaaactaccaataattaaaataggtgtcatcgccaccatt

SEQ ID NO: 26 - DNA, 1851 bp, Lactobacillus sake
gaaaccgttaacgttttaacgttaaccgttcttatctagaaagttaattttcgtagggttcgggtcgttttttgtttatcgtgatcattcccgcgcaggatgatcgccgccattactaaagtcggcttagcaatggcaacactcatgatttaaacgtggggcaacagacgcttagtctttgaaccacatttccacaacctgcaaaatttaaggagtgctgcttatgttagaggaacgtaaactcactaaacgcgattatttcgtggttagttcaatgctttttggtctattctttggtgctggtaacttgatttttccattacatttaggccagctagcgggacagaattgggtgcctgccgcgcttggcttcttagtcactggtgtgttattgccattattatccgttttagcaatcagtatcactcgttccgaaggtgtctatgatgttgggcggcctttaggtcccgtattcgcgctcttattcatgatcttgatccacggcaccatcggcccactttttggtacaccacggacagccaccgtttcctacacggttggcttagcgccactagtaccaaagaatttccaaacagttggtttattagttttctccgctgttttcttcggcttagccctctttttctcattagaggaaaataaaatcgtcaacaatatcggtaagttattaaatccagttttcttagtcttattagctggcgttttcttcttagcttttagttcaccaatgggccacgccggtgttcaacacgccaccgctgcttatacatcaggctctttcgtcaatggtttcttgcaaggttataacacgatggacgccttagctggcttagccttcggggtcaccgtagttaccgccgttcgcaccttaggcaaaaaacgcgctaacagtgtcgcgctggtcactgcccgtgccggcgtcttcgcaatggcaatgatcgccgtcatctacgttggtttgatcttcgttggcgcgatgtccttaggtaaactcaaagtttccgctaatggcggcatcgcttttgaccaaattgtcaatcattatatgggcgtcgctggtcaggccatcttagccactttgatcaccgtcacttgcttaaccacggcgatcggtttagttgcggcttttgcccaagactttcataaacattttcccaaagttagctaccgcacttggttagttctaactactctggcatcattcttgaccgctaatatcggtttaaatcaaatcatcgcttggtcaacgccaacgttgatgttcttatacccactatcaatggtcttgattttattgtcagtcgcttcaccactgttccacaaagacccagtcgtgtataaattcgttgttctctttaccgttgttcctgcttttttcgacatggtaactgcttttccagcggtcgtcagccaaagtagcttcggtaaagcgatgactggcttcgaacaggcatatttgccattcgccaactacggcttagcttggttagtccctgccttagtcggtgccgttttaggggttgccgtccacgtcttcaaacgtgttagcgcccgcaacaccgtttcacaaactaaataggtaagtcaaaaaaacgttcacttgcggtgaacgttttttattgccttgaatttatactttaagtgcaacaaaagagactcatgatatgaatccctgtaaaatggtgtttgcgaagacaaccatcgaagggagcacataatcatgagcccatacaaacatcttaccatgagtgaacgcgaaacgatattcctgatgcataataatcaggcttcactgacttcaattgcccaaacaattggtcgagcaacgtc

SEQ ID NO: 27 - DNA, 6346 bp, Lactobacillus sake
attgttctttatattttttatagaaaaaagttttatttgtaatgccaacttgctccataatatctcggataggttggttagtagacgttagtagtaatcgggcgttaattaaacgttggtaggttaataaacctgaaaatgatttccctgttttattttttaaaaggttgcttagataatttttattataacctagcttggcagcctctttagttagtgttatttcacgataatggtgttcaatatcatgtaaaacctcagtgactgtcttttgagaggagcttagtgtactagttggtttaatctgataatttctaacaagttcggttaataaaatttgaaggtaagatgaaattacttcgttatagaaaggacgttttttgacgtattcattaactagattttcgatcacagagtttatttgggagtgattattatgtttgaagaggatgaaatggttaatattttcttccccgtaactacgattaaggagtagtgaataaagtaggctttggctttttttaagattatttaaccagtcaattgaaatattagtattttgaaaaagaatattaatcataatatcgttagtgtttagtttttttatagtgtgtgtacagccaatatcaattaataataaatcgtgttggtgtaagagaatctcacgaccatcaacagtttgatgacagttaccagataccatgtaatttatctctaaaaattgatgtgtatgtgcgggataatctgcataacgattatgtttactaatataaatatccttattagcgaaaaaatatttattttgaagcgtaaaatcagaagatttattaagacgtgcatatgaaggaatatccgcaatataatggttattttttttctgctctttttcgatggttgtttcagccgttaaaagattaagaatactagagttcataatatttccccctgtgaaaatgatactttagttctctttaaatgacattgtaacattttgagcaatgaagttaaatataacttgtaagcgatttctaaaatgatattaggagatgagatctatgccagaaaaaagtaagcttaaatccgtaatagccgtttcgttaacaaactatttagattcaggctcaattgttgctggtgcaagtggacttgcattatggacaaaagcattaggacttagtagttttgaagtcggattgttaggggctctaagtgcgaatgcttttggtgccgctgtgggtgctttaattggcggtcgattatctgataaatatggtcgtaaatttatctatacgtacaatatgttggtttatatgttaggcgtcttaattgttatggtgtcatttaactttccgatgttattgacaggctttatggtaactgggatatctgtgggtgctggtgtaccagcatcatggacgtatattgcagaaacctctcaatcgagcaatcgggcacataatatcgggatatcacaatttgcttggtcgatgggaccagctattatttttgtactaggtgtagttttatcacctcttggccttttaggtaatcgaattatttttggctcgttgtttgtagttgcgctcattgcatggatgatgcaaaggggattggaagagtcttccgattggcaagaacaaaagcaaaaagaaaatgctagtggtattaaaccgcatccttataaagaattattctctaatatcgttaacgttaaaacgattttatatttaatcggggtttatgcattgtggaatttagcagcaggtgttcaaggatttttcatgccatacatctattcaactgttggtggtttaacaaatatgcaagctaacttattatcagcggtcgtttggattttaacttcaatgactggatatttcatctttgctaaattgggcgatcgcgttagtcatcgattaatgtacggtataggtgccttaatggcaatgttagcttgggttttaattacctttggcggtatgggatggactactctatggggatttgtattactttggggactatctgcaggatttggtgcacaagctttctacgcattatgggcaacagagttattcccaacacgttatcgtgcaggttctcaaggtgtaatgttttttgttgttagagcttcagctggtatttggtcgattattttcccaccaataatggcttctctaggatttaaatacgctggcactatgattgttatctttttagcaatttcattagtgattggtgttatctgggcaccacatacacgtggtaaatcattagacgaaataacagaagaacggtatggttcaaaaaaataatgtgaatcaaatactcaagagtattatgcgttaggagtgatttatttgaaaaattatattgcagttgatattggggcttctagtggacgattgatgttgggtcaactagtagatcaaaaattagaacttaaagagttgcatcggttttcgaatggattcatcaaagaaaatggccatgatcggtgggatattgaccgattaattgatgaaatatttaaaggcttagaaatcgcaaaaaaacaaggcatcgaaaacgcagttttaggaattgatacatgggcagtcgattatgtattaattggcgatgatggacggaaactacaagatccatgtagttatcgtgatagtcgtactgctaatagtattcaagaattaacatctgagtattctaaagaatatatttataaaaaaacgggcattcaatttttggattttaatacactataccagttatatgaagaagataatgagttacttgataaaactgatcgaattatgatgataccagattatattggttatgttttaacgggaaacgcagttacggaagtaacgaatgcctctacaacacaaatgatgaatcttcgtcaaggattatttgatgatgatttattaaagaaggtaaatgtaactgatgatcaatttccaaaattagtagatgcaggaacaaaattaggaaaattgtcacataaatggtatcgacaatatgatataccaaaatgtgaagttgtaacgacaacaacacacgatacagcttctgccgtcgttggaacacccggaaaaggtgatgactgggcatttttgagttcaggaacatggtcactgcttggtcgagagttggatgtacctgaaaatggtgaagaagcatttaaaaaaaattatacaaatgaatggggagcatacggtacttatcgatttttaaaaaatatcatgggactttggattattcagagtattcgaaaagaacaaaataataaatttagttttgcagaaatggctgaagcagctgaaaaggttgaaccatttcaacaatttattgatattaatgatgaacgttttaaaaatccaagcagcatgattgatgaaatcaaggcatattgtcaggaaacgaaacaaaaggtccctgatacaacaggtgaattagtaaacactgtttactcaaatctagcgctattttacgcgaacgaactacaaaaattagcgcggattactaagaaaccaattaacgttctaaatattgttggtggtggtagcaatgtagcattcttaaaccaactaacaagtgatttggcgtcagtgagagtcgaagctggccctggtgaggcaactgcaattggtaatattctagttcagatgattgcgctaggtgatataaaaaatgtatctgctgcaaggtcaattgtaagcgcttctaatggaattagacattttgttcctcgtgaaaattattatggagaagtgttagaagcatatcaaaattatattaatcgaaaaagtgagggaacaggcgaatgaaacgaattgcacagattatgtatgtttatccggatcaatatgatgaatatgaacgacgtcataatgaactatggccagaaatgaaggaagcattaaagaaatatggtgcgaccaattattctatctttttaaatcgtgaaaatggtgagttatttgcttatcttgaagtttcgaatgtagatcaatataacaaaatagctaatacagatatttgcaaaaaatggtgggtttacatggaaccacttatgaaaacaaacccaaacagtagtcctgtagcactagacttaaatgaagtattccacttagcgtaaaaatataggaggtttttgaaatgaataaagaagaaaatgttaaaaagagctatgaattagcaaaagaacgttatgctgcattaggtgttgatacagaaaaggcattagaagacttagctaaggtaaaattatcaattcattgttggcaaggtgatgatattcatggattcctattccctaaccaagaattgacaggcggtattggtgttagcgggaattatccaggattagcaacaaccccagatcaattgagttcagatttatctgaaggcctatcattgattccagggaagcataaagtacaattacacgctacttatgctgtaacggataagccaaaagatttagatgaattagaaccagaagattccaaatattgggttgactgggctaaaaaagagggtgttggtcttgacatgaacgggacattcttctcacacccaatgttgaaagatggcttcactttagcaagcccagataaaaatgtacgcgatttttggattgaacatggtaaacgaagccgtagaattgctgaatatttcggtaaagaattaggacaacaatctgttaataatttttgggtgccagatggttataaagataatccaattgataaaatatcaccacgtcgccgtttaatggcttcattagacgaaatcattaaagaaccaatggatgaaaaaaatacaattgaagctttcgaaggtaaattgtttggaactggtgttgagtcatacactgttggctcacatgaattctatgaaaattatgctttaacacgtcaaaaattatggacaattgatgctggtcacttccatccaacagaagatgtttcagataagttttcggcattcttaccatttggtaaaggtttaatgcttcatgtttcacgtccagtacgttgggatagtgatcatgtagttatctttgatgatgctttaatccgaattatgcgttcactggttagagatgacttattagggcgtactaatattgggttagatttctttgatgcaactattaatcgtgtttctgcatgggtcgttggtgcgcgtgcttctcaaaaagctattctacaagcaatgttggcaccaattgatgatttaaagaaagcagaagaacaaggcgactatacaaaacgtctagcagtgacagaagaattaaaatcatatccatttggtgctgtttgggatgaattctgtgttagaaataatgtaccagttggagaagattggttggataatattaaagattatgaaaataacgttcagttcaaacgctaatcaaaggggattataaaatgacgaagtttatagattcaccatttgttgaagaagtaaaaaaaattacgtatcgtctatttgatcatggatgggacgagcgcaacggtggtaatgttagctatcgaattttacctgaagaattaacaggatacacagatattgaaaccgttaaacggaccatcccaattgattttaatggtaaagatttagcaggcatgtatttcttagtaacagcaacaggaagatattttaaaaatattattgattttccagaacgtgatttaggactagttagaattaatgaagatggtactagtgttgacttaatgtggggatattcagatggtgctgcacctactagtgaatttccaacacatctaatgggacatcaagtacgtttaaaaatggataataatcaaagagttgtcatgcactgtcatccaacaaacctaatagccatgacatttacacaaaagttagatgaacgtgaaatgagtcgtatcttatggaagatgcaaacagagtcattagttgttttcccagaaggcattggtattattccttggatggtaccaggaacggttaagataggggaagaaacagcgcataaattagaaacttatcgaaccgttatttggccacaacatggtatttttgcgacaggtagcacaatcgatgaagcatttggattaattgaaacaattgaaaaagctgctcaaatttggaccattattcaaacccaaggcggacatattgaacaaacaatcacggatcaagatttaaaagacttagcacaggcgtttaacgttacccctcatgaaggttatttggactaaatttattgggtaggaaggtatttaaaatgaaaattaaagctgcagttgttgaagaaaaaggtggaccctttgtcattaaagataat

SEQ ID NO: 28 - DNA, 1984 bp, Lactobacillus sake
tacatctattttgcagcaaccgatacgaccgatattgtaaacggtttattcacacatagaatgtattgtattgaatgcgctgcagataatcctatgaactccgaagatgattgggttgaaaaaggacaaatcgtcactgataaagatacattttgcttagatgccacaacatttgaacataacaacaagctttattatgtctgggcacaaaaggacaacgatatccgcggtaattctaatttatatattgctgaaatggcaaacccctggaccctaaaaacaaaaccagtcatgctttcaaaaccagagttcgattgggaaacacgggggttctgggttaatgaaggacctgccattttgaaacgtaatggcaaaattttcttaacttattcagcaagtgctactgatgaaaattattgtatgggtatgctaactgttgatgattctgccgacctactcgatcctactaactggcataaagaagctgaacccgtgtttgctagtgatttaacaactgaccaatttggccctggccataactcattcactatttcagaagacggcttaaccgatattcttgtttatcattgccgcgactacactgatatcaaaggtgacccactttatgatccaaatcgccatactaaagttcagccattttcatgggataaatcaggcatgccacgtttcgataaacccgttccttacaattacaaataaagggagattattagtatgaatgcaattcataaagaaaataaacacttttggagtttcgcaagtactcatttcacctacttctttatctgggctattgtttacggctatttaacactttggttagaacaagtcgctcatttaaatgggactgaatcaggctttatcttttcatttatggccggtatttcgttgttcttccagccagtattcggaattttatctgataaactagtcttcaaaaagaacttattagtgaccattacattagccgctattttggtcggacctttcttccaatggttattcttgccaatcgtccataccagttcattcttagctgctattattgctggtttatatctcagttttatcttaaatggtggcgttggcgttgttgaacaatacgttgaacgtgctagtttagtcaataaatttgaatacggccactcacgaatgggtggttcactagctggtatgacagcatcccttattgccggtcgcttattcatctggcaacctaactcaattttctgggcatgttctgtttccgccgttatcctaacttgtcttgttctattcagtgataaagtcaacgttagtgatttacaatccaatccagatactgattcagacaatgaatcagctccgttagataaaaaaacaattctttctatctttaaacttaaaaatttctgggtactttcattattctacatgggtgccgctgcaatctatgatgtttttgatcaacagttcattgtttacttcacaacgttctttaaatctgctagtcaagggaccattacctatagttacatggcttcagcccaaactgctattgaattactattgatgattccaatgccatttattatcaacaaaatcggtgctaaaaaaggcctaattgcatttggttttattacattcatccgtattttcgggagtgcaattgcaccaaactgggaattattagtcttctttagattattagctggtttagaaatgccattactcttggtttcaatcatgaaatatatcaccggcgcatttgatattcgtttgtatgcaacggtttacttaatggcttccaattttgctaaacaaatttcagttttcatcttctcaacagttgccggtaaattatacgatgtcgttggttttcaaaaaacatacttcatcctttcagctatcgtttttgtcattactttagtcgcagcgttcatgctcgaaaaagaacacccacttgttgaacaaactactggtc

SEQ ID NO: 29 - DNA, 2511 bp, Lactobacillus sake
atgaaaaaaataatgaagttagtaatattcggtattttgatagcgctattattggtgatttatgtagagaatgtgacggccggacagagagaaaccgccacgacccagaagcaatttacgaaaacacaaacgattttaaaaaatacaacgagccaagcaaatcagaaaagtaatactcagaaaatgtcgccggaaacggctgaagcagtcggcgataaaaagctcgccaaagctatgacaattgatgaaggtgttcagaattgggcggtagtaccgcagtcaacgattgcctatgtcgacttgggtaacggtcgtcgattaggctacacttttggtaaagcgagtgtgagtcggtcagatcgttcgattttaaaattggggcaaacacctaaggccaatatctttataaataataatggtcggatagattctgccttctttggggctaaacaaaatttgattacgtcatttgattatgcaattcatcccgatcggtcaaataaaaatccatccatgataaatgaatcaattggtgagcggttaaaatcatatagcatctataaagggtcaaccagtgatggtagtattgcactcaaagccgttggtgcagttgaagttaataagaaaaattatgaagcaacgttattaatgcgtcccaatcctgggaaagccgttattcaacaagagctatatctggcgagtaaagaggatacgaaacatgaggccggcatttatttagcgaaggatttattggtggattatgacgataatattccggtttattcacaaggtaataatgctggattatatctgcaaagtaagaaatacaaagtttttatcaatgttaaagtgccggatggtccgcaacactacggtactggtagttatcttgattcatatagttatgccttatcgccatataaaggaacggattttgatagccctggactagaaagcgataatctaaaagagggtactgctgtttttaaaaagctggattctacgtacagtaataagtggtcttggcaaacttttgagccgaacactgtatatcattatcgttctgatattggcttgaccgaatcaggtgtggttagcccggatgcggttattaaatatacaaataagaccagtgacagcggaaagaatcatgtgaacgatcagctgacagttgaatcgatggcgcataataatggttataacagtgactggcaaggcgtgacggttagggtcgacgttcctaaaggcttaacaatcgattcggactcgattaagttgaaattagctgatgagacaatccaggcggttgatgcgagtcattatgatgcacagactggtcaagtgagtgttaaattgccgaaagctttggcggaaaatgaatggacaagcgttctgtacgatgcccaaatcaataaaagtgcggcgggtacgacgatgacgccgaaaatgatcgctaagggtgaaaatggctttcctaacgaagcatcagatgacgttcagattccggttcaagatcaaccaaccgccatcgaaaaaatggtacgtaatgcagcagatccggatggtgattacggacaaacggcggaaagtgcggtcaagggagtacttgattaccgtgttgttctgaaggttaaccggtctgatggtggtcttgtagaaggaacgctcagcgatgaactgccaaagggattaacgctagttcctaattcggcaactataaaatacggacagcagacaactacgaaatcagttagtgacatcgggaaaattagtctggaatcaatggtagcaggtgaaacagcaaccttaacttatcgggcacaggtcgctgatacggttgatgacggagccttattaaagaatacggcacaggtgactggtaaagaagtcaatggcactgcgctaacgattgatgccagtgcttctgtgaagattaccaaacctaaaatcggaaaggtaatcttcaaatatcttgatcgggctactggacagccaatcggcgatcagcagattacaattaccggaccaatcggtaaacgtctttcagaagttaagtcggatgaaattcaagtcagtggtgggcaaaatattaatcgaatccgaccagcttatatcgatagttatgtgccggtcgactttaccgccgatggtaatgccagtgtgccgacttttgataaccatgttcgggatgttgatccggtctttgatgagaccgagacagtctatcttttcaggtatgagaagcagcgactcgaaataatggcccacccagtgaaattctcatttggaaaattctcgaatacacaggctgaacgtacgtattatctaccaacggctaaggatcaaaatgggcagaaaaagccatattcgattggaattgcggattactggggaatcaatagttggcaattaacggtgagccaacctgagcaatttgaaggtacctatctcgatcaagataagaaaccacagcgcacagctctcgacgacgcccgactgatttttaataat

SEQ ID NO: 30 - DNA, 2757 bp, Lactobacillus sake
tacattgccaaaaggaactgctagtccgattggtatgctgatttacaacgttttaggtttaattatctttatgggatttgcttggatattctgggcacgttacgaaaaaaaacaaaaaaaggtggctgaatcataatgaaattatgggaaaattatcaactcgatagtattaatcgtgaaccggcgcgtgcacactttttaacatttccatctaaagaagcggcgcaactcaatcaaaataagtacacacatgctttcaaaaatcttaacggcaattggcaattcttattcttacaggcccctgaatatagccctaacaacttcttcaaacctgactttgatacttcagggatgacaactatcaccgttccaggttcatggcaagtacaaggatttggtaagatgcactattctgatttgtggtataacttccccatcaatccgccgtttgtaccaacgattaacccaactggcatttacaaacggtcttttacaattgatgaatccttcagcaatcaaaaaatcattttgcgttttaacggtgttgattcagcctttgaggtctgggtcaatggcgaatcggttggttatagtaaaggtgcccgcaatgaggcagagtttgatatcactgatcttgctaaaatcaatcaaacaaacgacgttactgtccgcgtttatcaatggtctgatggtacttatttggaagatcaagatatgtggtggctcagtggtatttatcgggatgttgaattactaggtgtccctcaaaacggtatcaatgacctaaccgtcgttgcggatttagatgctagttaccaaaatggcttattatctgtggaaacgacattccgccaacaaaccgatcaaactgttacttttgaacttcggaatcctgctggtaaagtcgttttagaggctagccaacaagttactaatgcaaaagcgaccctcgctgctactattgatgccgttgatgcctggactgctgaaacgccaaattgttatgatttaacgattagtatttatcaagctgatcaattaattgaagtcatcccacaaacagttggctttagaaatattcatgtcgaaggtgaaacgttcctagtcaacggtgtcgctattaaattcaaaggtgttaatcgccatgactataacccccataatggccgtgttgtttctaaggaagaaattgaacaagatattattctcatgaagcaatttaatatcaatgcgattcgcactagccactaccctaatgcctattatctctatgatttatgcgaccaatatggcatgtatctgattgctgaagctgacgttgagtgtcatggctttgagttaactcagaaatacgattggatcactgatgatccacaatgggaaacagcttatgtatcacgtttaacacgtatgattagccgtgaaaaaaatcatccttcaattctgttctggtcacttggcaatgaatcggattttggtcataattttaaaagaatggccgctgtcgcaaaagaaatggacccaactcgcttagtgcattatgaaggcgattttgaagctgaagttgttgatgtgtattccacaatgtatacctggattgaacatcctactaaaaagctactcatgaaagacatcatcgaaaatagtaaaaaacctcatctcttatgcgaatatgcccatgcaatgggcaatggccccggtaacttaaaggattatcaagatttattctacgctcacgataaactccaaggcggttttatctgggaatggttcgatcacggtattgagtcgcagactgcatctggtgagacctattaccgctatggtggtgattttggtgatgaccccagcaatcgtgacttctgtattgatgggctcctaatgcctgatagaaccccatctcctggcttatatgaatacaaaaaagtcattgaacctatcactacatctgttattaatttagatgctggagaattcgagttacttagtcgatttgattttgctaacctcaatcaattccaactcgtttatactgtgatggcagatgatagtattcttcaaagcggcaccttagcactcccatcaattccagcccgccaaaaagcaacgattcaagtaccaattgatctcgactttactgctcatcccggcacaacctacctattgaatttctcttatcaattagcaagcaataccccttacgcaccagctggtcacgaagttgcaacggctcaatttgaactaccaattcatcaaatcggacttcctattaaaccggttggaacactcacaactcaggaagatggtgcatttcttcaagtaactgggaccaacttctccctcacttttgataaggtactaggtgccattacgagcttaacacgcgacggtatcgaactcatgtcaaaagggccacacctcactttctggcgagcaccaatcagcaatgatatggaaatcgtggatgagatgaagaaaaaacaattccttcacttagaacatgaaaccgttcaaaattttgactaccatcaaaatggccaacaactgattgtgactgttgagacattaaacggaacaaccaatagcgcttggcattacgattgccgttatgaatacaccatctatccaagtggcgatattcaattcaagctcagtggcataccgggtggcaaattagatatggcacctgaaatgcttccaagattaggcgttgtcttaaatctgaaccaatcactcgaacaagtccgctacttcgga

SEQ ID NO: 31 - DNA, 1680 bp, Lactobacillus sake
atgttgatactgatattaataagttctgtaattattaatgagaacgttgtcaacgcagtccaaagacaggataattcatcagaaatcaaggtgccaacagattcaaaaacaagtagtcactcagaacaatcagtgagaacccaatccaaatctgaatctaagtctgaatctaaaacgacgtcatctgaaaagcattcttctgagaagacaacacagtcctcaagtagtcatgagggtaagtctgtgattcagaatcaatcacgagatacaataatgccgacagctactattgatgatggcgtgacaggttggcaaccggcaaagcaaacaacgattgcaagcattgatattgataaagcggctaataaaagccttaattatacttttggtaatagtgatgggaccttaaagtttgatcaaggaaatggtgggataataacgaataacatgccgccaagaatgaacgttttcattaaggataatggcaagattgtcgagtcttcattcaaatcaacatctgggatgggggctcgtatgactattcactcttaaccactagaagttccacggcaccaacgattaaaaccacatcgacatcggcacagggatcactgacaggtgtgcaatattatcaagccaaagacgccaaagggcagacaattttaaaaatggtggggcaatatacaaccatagtggggcgtgtccccagtttcacacttgagtatctattacggccgtcactagaaaaaccatctgttcaacaggaactatatatttataatgcgacatcggcagcaattgatggcgggatcttttttgctaaagatacttcgttaaatggtaacgatggcataccagtttctgcgcaagggaataacgctgggatgtacattgccgatggtaaatataaactattccttaatatgggtgttgaggatggtccagccaaatttaacgcgatgaattatcaccgaaatggtggatataacttatctgattatgcaatgaatgattattcgccagccaactttgatggtacgggtatcgaagttaaaaatctaaaagaaggggctactgtttattcaggaagtgattctgcgtatagcgctaagtggaattagcaaacttttcagccagataccgtttatcattttagaaatgatttagggattgcgactgcgggcttggttgtcccagaagcggcagagaactataaaaataaaacgacaagtgatcaaaaaaatcacgttcaagataatattactatcgaaatgaaagcgcataatttaggctatagtagtgaatggaaagatattaacgtaacgtcaaaaataccagatgaactggatattgatcctagtacaattaaagtaactctgaataatggcgatcaagtagcaattgatgctagtaagtataaccaaacgactagaatgttaacggttagattacttaaaaatttgtccgaaaatgagtggtcatcgatcagtttcgatgccaaaattaatggtaaagcttctggaaaaacaattaatagtagcatggttgctaagactgaaggtgggactggcagttttgatacggctacggcgagcacgaaaatcccagtcgaagttgaacctgtcgaaattgaaaagaaagttttaaatgaaactgaaaaagaaacgacgtaccaaaaag

SEQ ID NO: 32 - DNA, 1680 bp, Lactobacillus sake
atgttgatactgatattaataagttctgtaattattaatgagaacgttgtcaacgcagtccaaagacaggataattcatcagaaatcaaggtgccaacagattcaaaaacaagtagtcactcagaacaatcagtgagtacccaatccaaatctgaatctaagtctgaatctaaaacgacgtcatctgaaaagtcttcttctgagacgacaaagcagtcatcgagtagtcatcgagataagtctgtgattcagaatcaatcacgagatacaataatgccgacagctactattgatgatggcgtgacaggttggcaaccggcaaagcaaacaacgattgcaagtattgatattgataaagcggctaataaaagccttaattatacttttggtaatagtgatgggaccttaaagtttgatcaaggaaatggtgggataataacgaataacatgccgccaagaatgaacgttttcattaaggataatggcaagattgtcgagtcttcattcaaatcaacatctgggatgggggctcgtatgactattcactcttaaccactagaagttccacggcaccaacgattaaaaccacatcgacatcggcacagggatcactgacaggtgtgcaatattatcaagccaaagacgccaaagggcagacaattttaaaaatggtggggcaatatacaaccatagtggggcgtgtccccagtttcacacttgagtatctattacggccgtcactagaaaaaccatctgttcaacaggaactatatatttataatgcgacatcggcagcaattgatggcgggatcttttttgctaaagatacttcgttaaatggtaacgatggcgtaccagtttctgcgcaagggaataacgctgggatgtacattgccgatggtaaatataaactattccttaatatgggtgttgaggatggtccagccaaatttaacgcgatgaattatcaccgaaatggtggatataacttatctgattatgcaatgaatgattattcgccagccaactttgatggtacgggtatcaaagttaaaaatctaaaagaaggggctactgtttattcaggaagtgattctgcgtatagcgctaagtggaattggcaaacttttcagccagataccgtttatcattttagaaatgatttagggattgcgactgcgggcttagttgtcccagaagcggcagagacctataaaaataaaacgacaagtgatcaaaaaaatcacgttcaagataatattaccatcgaaatgaaaacgcataatttaggttatagtagtgaatggaaagatattaacgtaacgtcaaaaataccagatgaactggatattgatcctagtacaattaaagtaactctgaataatggcgatcaagtagcaattgatgctagtaagtataaccaaacgactagaatgttaacggttagattacccaaaaatttggccgaaaatgaatggtcatcgatcagtttcgatgccaaaattaatggtaaagcttctggaaaaacaattaatagtagcatggttgctaagactgaaggtgggactggcagttttgatacggctacggcgagcacgaaaatcccagtcgaagttgaacctgtcgaaattgaaaagaaagttttaaatgaaactgaaaaagaaacgacgtaccaaaaag

SEQ ID NO: 33 - DNA, 4698 bp, Lactobacillus sake
atgaagggtattattttagcaggtgggtcaggtactcgcytgtatccaattacaaaggcagtwtcaaarcaacttattccrgtttatgataaaccaatgatttattatccaatgtcarytttaatgttagctggcattcaggayattatgattatttcmacaccaasygatacaccacgtttcgaagaattatttggtgatggycatgatytgggyttaaatattgaatatgccgtgcaggataatcccaacggtttggccgaagcattcattattggggccgattttatcggtgatgattcagtctgtttaatcctaggtgataacatctattatggtggtggcctatcagaaatgctacaaagagcggctcaaaaggaatctggtgcgacggtctttggytaycatgttaatgacccagaacgttttggggtcgttgattttgatgaaaatatgcatgcgaaatcaattatcgaaaaaccrgaacatccagcaagtaactacgcggtsackggtttrtayttctaygacaaycaagttgtygagattgctaaaaatatcaaaccatcaccacgrggtgaactcgaaattacmgatattaacaaggcttatctygaaaaaggycaattagatgttgaattaatgggccggggctttgcktggttagatactggcactcacgattcattrcatgaggcyagcagttttattgcaaccgttcaaaagcgscaaaayttaaargttgcttgtttrgaagaaattgcttaccgaatgaactatatcgataaagmraaagtgattgaattggcrcaaccgcttaagaaaaatgattatggccaatatttattgagattagttggtgaaaaaaatgattagattattattcttagggacacaggatattgatgataaattatcgtggtctggcaccatgtaccacatgacacaagcaattaaaaaagatgaaaactatactatatcttctttaaaagttccggagaaaagttttttggataagttaagaattaaaatgcttgctataccaagaaaaatttttagcccagggaaaaaatatgttagtaatgtagacgtacaccaagttcgaattaatggaaaaagattaacaaaactattggcagataaagttgaaaaggaatcagtagatattatatttgcaccagccatgagcacatacttagctaatttaacaactaatattccaattgtatatttgagtgatgctacttttgcactcatgaataattactactggtttggattaagcaataaagaagttagggatggaaatagagtagagttggctgctcaagaaaattccacagcacttattttttcaagcgagtgggcggctaatagtgctattaatgattataaaagttcaatagacaaggttaacatagtaccgtttggagcaaatttagcgactagtaatttaataaaaaataaaaggattaattcaacaagagaggttaaaattcttttggtcggtaaggattatgaacgaaaaggaatttttattgctgaacagataattcaaaagttgaatattaattctgatatccgatacaagctaattgtagttggagttgaccgagaagatactgataatattcattatgctggagtattggataaaggcaatgaaacggattataaaaaattgatgaactattaccatgaatcagtattgttcttactaccgacaaaagcagaggcagccggaattgtttttgctgaagctagtatgtttggactaccaactctgacttttgatactggtggtattggtgaatatatcattaacggcgtaaacgggtaccgaatatcacttgatagtcaagatattgtagcttcatttacggataaaattgatgaaattgtacatggtgaagtattatatgagcgtttatccgtaggtgcggtggaacaatatcagacgcgattgaactgggatcattggatgacagaatttaataggattgttrasawtgtamwmrrwgakkagrryaakmaathatgggaaaattaaaagtaacaacaactaaactacaagacgttaaaattattgaaccagccgtttttggtgatcaccgwggvttctttacrgaatcatattcagaycgtgactttaaagaagcyggrattgatatcrattttattcaagayaaycattcaytwtcaackcaagcmggygycttacgtggyttrcatttccaacgkggcaaagcygctcaaacgaaaytaatycgrgttgtkacwggygcygttttagacgtcatygtrgatgtgcgtgcyggytcmccaacttayaagcaatgggaaggytatatyctttcagcaagtaaycaccgtcaattattagtrccaaagggyttygcycacggttttgtgacgttaaccgataacgttaacttcttatataaatgtgatggttactacgatgccgaagctgatggtgggatttcatttaagaccccagaattaaacattgactggccaatcgattttgaccaagcaattacatcagaaaaagatgcaaaccaaccaacctttatagaattcgaaaaagacaacccatttgtttacggtgaaatttaaggagactctaacttatgaaaaatattattgttaccggcggcgccggttttatcggttctaattttgtccactatgttgttaacaatcatccagaggttcacgtaacggttttggataagttaacttatgcagggaatcgcgctaacttagctggtttaccagcagaccgtgtagaattagtggttggcgatatctgtgacgctgaattagtcgataggttggttcaaaagacggacgcggttgtgcattacgctgcagaaagtcacaatgataactccttaaaggatccatcacctttcattcaaactaatattattggctcatatactttaattgaagcttgtcgtaaatataacgtgcgttatcatcacgcttcaactgatgaagtttatggtgatttaccattgcgggaagacttaccaggtcagggtgaaggtgctggtgaaaaatttacacctgaaacacgttacaatccaagtagcccttattcatcatcaaaagccagttcagatttattagtgcgtgcttgggtccgttcgttcggtctacaagcaactatttcaaactgttcaaacaactatgggccttaccaacacattgagaaatttattccccgtcaaattactaacgtgttaagtggcattcgtcctaagctttacggttcaggtcaaaacgtgcgtgattggattcatacgaatgaccactcaagcgctgtttgggccatcttaactaaaggtaagattggtgaaacttatttaatcggtgccgacggcgaacaaaacaacaaagatgtcttggaattactcttagaattaatgggtcaacctaaggatacttacgaccaagttaaagatcgtcccggtcatgatttacgttatgcgattgattcaacgaagttacgtgaagaattaggctggcaaccagaatttactgatttcaaatcaggattacaacatacgattgactggtatcgtgataacgaagactggtggcaagcagaaaaagcagcagttgaagccaactatgctaaaaatggtcaataaatataactaaatggaagtaggcgagcgcctactttttttaaggagaagagttaacaatgaaaattttaatcacgggtgccaatggtcaattaggaaccgaattacgtcatttattagatgaaaatcaaatcgaatatgtaggaactgatgccgctgaattggatatcacgaatgcagaagcagttaatgcttattttgaagctaacaaaccggaattagtttatcactgtgctgcttatacagccgttgatgcagccgaagaagaacctggcaagtcaatcaactacaaggtcaacgtcgttggtacacaaaatattgctaatgccgcagaagtagttggcgcaaccttggtttacatcagtacagattatgtcttcgatggcactaacgaccaaatgtatacagaagaaacacctgctgcacctaagaacgaatatggtcggacaaaattagctggtgaagaagctgttgctaacacgatgagcaagtattatattatccggacatcatgggtctttggtgaatatggtaagaactttgtttatacgatgttgaacctggcgaagacacacgatcatttaacagttgttagtgatcaagtcggtcggccaacttggactcggacgttggctgaattcatgttatattcagttaaaaataatgtaccttacggtttatatcaactttcaaacgacaattcatgtacttggtatgaattcgcgagtgaaatcttgaaggataaagacgtagaagtaagtccagttacatcagatgaatacccacaaaaagctttccgtccaagacattcaatcatggatttgacgaagattaaagcaaccggctttgaagtaccaacatggcaagaagcattacaaaaatttatggcaaagattgactaatatagatagtatataaaaaggataaagtcgactgactttatccttctttattagattaacaatttactaaactttttctgcaccctaaatttaatattattagccgttcgccgtgtgatgtttaagtccgcggcgat

SEQ ID NO: 34 - DNA, 2549 bp, Lactobacillus sake
atgaagggtattattttagcaggtgggtcaggtactcgcttgtatccaattacaaaggcagtttcaaagcaacttattccagtttatgataaaccaatgatttattatccaatgtcaactttaatgttagctggcattcaggacattatgattatttccacaccaactgatacaccacgtttcgaagaattatttggtgatggtcatgatctgggtttaaatattgaatatgccgtgcaggataatcccaacggtttggccgaagcattcattattggggccgattttatcggtgatgattcagtctgtttaatcctaggtgataacatctattatggtggtggcctatcagaaatgctacaaagagcggctcaaaaggaatctggtgcgacggtctttggctaccatgttaatgacccagaacgttttggggtcgttgattttgatgaaaatatgcatgcgaaatcaattatcgaaaaaccggaacatccagcaagtaactacgcggtcactggtttatatttctacgacaaccaagttgtcgagattgctaaaaatatcaaaccatcaccacgaggtgaactcgaaattaccgatattaacaaggcttatctcgaaaaaggtcaattagatgttgaattaatgggccggggctttgcttggttagatactggcactcacgattcattgcatgaggccagcagttttattgcaaccgttcaaaagcggcaaaatttaaaagttgcttgtttggaagaaattgcttaccgaatgaactatatcgataaagcaaaagtgattgaattggcacaaccgcttaagaaaaatgattatggccaatatttattgagattagttggtgaaaaaaatgagagattagtaaaagaagattaggggtaagcaattatgggaaaattaaaagtaacaacaactaaactacaagacgttaaaattattgaaccagccgtttttggtgatcaccgtgggttctttacggaatcatattcagatcgtgactttaaagaagctgggattgatatcgattttattcaagacaatcattcattatcaactcaagcaggtgtcttacgtggcttacatttccaacgtggcaaagccgctcaaacgaaactaattcgggttgttactggcgctgttttagacgtcattgtagatgtgcgtgctggctccccaacttataagcaatgggaaggttatatcctttcagcaagtaatcaccgtcaattattagtaccaaagggctttgcccacggttttgtgacgttaaccgataacgttaacttcttatacaaatgtgatggctattatgatgcagaagctgatggtgggatttcatttaaaaccccagaattaaacattgactggccaatcgattttgaccaagcaattacatcagaaaaagatgcaaaccaaccaacctttatagaattcgaaaaagacaacccatttgtttacggtgaaatttaaggagactctaacttatgaaaaatattattgttaccggcggcgccggttttatcggttctaattgaaggagaataataaattatgaaaattttaatcaccggtgctaatggtcaattagggactgaattacgtcacctattagatgaaaatcaaattgaatacgtgggcacggatgctgctgaattggatattaccaatgcagaagcagttaatacttattttgaagctaacaaaccagaattagtttatcactgtgctgcttatacagccgtggatgctgctgaagaagaacctggcaagtcaatcaattacaaggtcaacgtggttgggacgcaaaatattgctaacgctgctgaagcagttggggcaacattggtttacatcagtacagactatgtcttcgatggcactaataatcaaatgtatacagaagaaacaccagcagcacctaagaacgaatatggtcggacaaaattgactggtgaagaagccgtggctaatacgatgagtaagtattacatcatccggacatcatgggtctttggtgaatatggtaagaactttgtttataccatgttaaaccttgctaaaacacatgatcatttaacggttgttagtgatcaagtcggtcggccaacttggacccggacattggctgaattcatgttatattcagttaaaaacgacgtcccttatggtttataccaactttcaaatgacaattcatgtacttggtatgaatttgcgagtgaaatcttgaaggacaaagctgttgaagtgagtcctgttacatcagacgaatatccccaaaaagccttccgtccaagacattcaattatggatttgacgaagattaaagcaactggttttgaagtaccaacatggcaagaagcattacaaaaatttatggcaaagattgactaatatagatagtatataaaaaggataaagtcgactgactttatccttctttattagattaacaacttactaaactttttctgcaccctaaatttaatattattagctgttcgccgtgtgatgtttaagtccgcggcgat

SEQ ID NO: 35 - DNA, 2157 bp, Lactobacillus sake
atgatagattgtaaaatatatattccgcaggttgatgagatggattgtggcgtagcagccttagcgatgattcttaagcattatggttcttcagtttcgctggcttatttacgtaatgaggctaaaactgatttagaaggaacaacggcgcttggactagtgaagacagctgagaatttaggtttcgaaacgaaggcaattcaagctgacatgagtttgtttgaagtcgcagatttaccatttccttttattgcacatgttctaaaaaatggcgagctattacattattatgttgtactagggataaaaaaagatgccattataattgctgatcctgatccaacagttggtgttactaaagtggctcaatcacagtttaaagcggaatggacaggtgttgctttatttattgcaccaaagccgaactatcaaccagttaagcaacaaacaaaagcgagtttattctcatttgtcccaaccttgataaaacaaaagcggttggtgattaatattacattagcagctttactcatcacgataattagtattttgggttcgtacttcttacaaactgtaattgatacgtatattccaaataatatgcatagtacattagcgatagtcgcgattgggttaatcatattttatggttttcagtcaatttttacatatgcacagacctttttactaacaatcttggggcaacgattatcaatagaaattattttgagttacttgaggcacgtttttgaattaccaatgtcattttttgcaacacgaaagacaggggaaataatttcaagattcaatgatgctaataaaattattgatgcactagcgagtgcgattatttcaatttttttagatattactgtggtcgttattatgggcagtatcttgatgattcaaaatacaactttattttggttaactttagcggttattccaatttatgttgcaatcattactttatttgcgaagccatttgaacggttgaatcaaaaggaaatggaaagtaatgcagtattgaattcagcgattattgaagatctacatgatattgaaacaattaaagcattaacaagtgaacaggaacgttatcaaaaaattgatacggcgtttattgattacttaaaaaagatttttagctatgttaaggctaatacactgcagcaggcgattaagttatttattcaattaggattaaacgtctttgttttgtgggttggtgcgctattagttacccataatcggttatcaatcggccagttaatgacatataatgcgctattggcttattttgtaaatccattacagaatattattaatctacaaactaaattacaaagtgcgcgggttgctaacaatcgtttgaacgaagtgtatctagtagactctgaatttaaggtagatcgaccggttaaatctgagacacaattgataggaccaattgaattacagaatgtgagttatcaatacggttatggtcaaaaggtgttagaagcggttaatctaagaattatgccaaatgaaaaactgacaattgttggcatgagtggatcggggaaatcaacattagttaagttattagttgatttttttgaaccaacggagggaaaggttctttttaatcaacaagatacgcagacaattgataaaaagacattgcggcagttttttaattatattccgcagactccttatattttttcaggaaccatctgggataatcttaagttaggtaatcgtcccgggattacacaagctgatatcattcatgcatgtgaattagcagaaattaagaccgatattgaaaaaatgcctctccaatttgaaacattattggatgaagatggtaatactttatcaggtggtcaaaaacagcgattaacgattgcgcgggcactcctgtcaccagctcaagtattaatatttgatgagtcaactagtgggttagatacgataacagagggactattggttgatcatttaatggcattagaagaaaaaacaatcattttcatagcgcatcgattagcgattgctaagaaaacgaataatattctggttttagaaaatgggagagtggttgaaactggaacacatgaaatgttagtgtcagcacagggacggtattatcaattgcttaatcattag

SEQ ID NO: 36 - DNA, 2202 bp, Lactobacillus sake
ttgaatgcgtctaatgcgcaaggaaaagcgctggctggcgttcaactaaagctcaaactgatgggttctaaagaaagttatgagcgaacagttacgacaacggcaacaggtaaagctactttggcagctgtcccagtaggcgactatcaagtgacgcaagaatcaacggtgatgggttatcagccagccactgctgttcagtcattaacaattagtaacgaccatcagaataccgtagattggcaattgcaacgaatggtagcggatgttacttttagagttcgcgatgccgcaacgcatcaaccgctagccaaagctgcttttactttaacgacagcaacgccggctgataacggtcaaactgtttttgtttcgcaaaagacgaatcaagatggtgaagtgaccattaagcaattaccaacaggcaaaataacttacgagcaaatggccaatgctgcgggctacgagccactagcagcgattcaaacggcgattgtgggtgctgatggggcacaaaacactgaagtaacagttgataatcagcggctaaaagtgactgaaaaacagcaaattatcgtgcataaaaccaatcaacaagggcaagggttagacgatgcggcctttaaattaactaatctggcaactgggcagacacaaacgcaaaagacggtgtccgggcaactacactttactgagttgaacccaggacggtatgagattcaggaaacaaaagcaccaactggctatcagttggatcaaacacctcagtttgtgacgattaaagtccatgaaaaacggttataccaagtccgctttgctgataaacgtgaagtgactgcgccaatcagtccattgcgcatccagattttagataatcacttgcaaggcgttgctggtgttttactgcgactaaccgccgatcaaccggatgaccaaggtcagaccgtttgggaattgacaactgatcgattcggacaggctatactccccaatgcaagtacggggcactatcgggttgaggttttacaagtaccgaccgggtatcaattgtcgtttgatcaaagtcaattagatgtcagccgctatggtgaaaatcagttacaattacaagctaatcgaattgagatgccattgcaaaccttaacgattaataaaaccaacttaaaagggcaacctttatcaggtgctgtctttaaagtcgagtcactgaaaactggtcagatgactaaagttgaaacggatcaaaccggtcgggctgagttgcgggaccaaaaaccagggcgctatcgcgtaactgaagtggaagcaccacttggttaccggttagcaactgaacctagaattgtagcgttgtctgaaaaatcaccgcgagcaacacaactaacggtaacagatgaagcacaaatggggcagttactgattaagcatacaactaaaaagggcgcaccgattgcaaaggcctactttgaggttaaggatcaaagtggtcgagaagtcggttattatcaaactgatagtcaaggacagattaaattaacccagctagcagtggggcaatatactgttcaagaaatcaaggcaccaactgggtatgaaattaatccggcagtgacaaaggtggccattacggatcgcaaaacggcaacggtcgccattaaggctgagcaacaagcacccgatgttaaattggggagttttgtattgatcgatagggatcagaaaacgcagttagcgattgctagtgcaacttatcgattggaaacgttagctggtcaggcggttcggccagaaattgtagtgggtgcaactggtcaagtcgtagttaacgacctcacacctggtcaatatcgactcgtacaattgacggcggcacctagctatcaaaaacaaactaacgatcaaatcatcgagattaaacaggatgcgcaacttaaacaagtgacgattgaaagtcatcagtcacaagggacagtcattgtaaatcaagtagatggcaatacgaaccaagctttggttggagcgaaatatgaattacaaaatcaaagtggtaaagtgcttgtcgaaaacttgaagagtgatgaacatggtcaagttcgcatcacacatctcgatcctgatacgtaccgtttggtgcaagtatcggcaactaagggctatgacccgcttaaaaagccgattgtctttacgataaccaatcgctaa

SEQ ID NO: 37 - DNA, 1020 bp, Lactobacillus sake
atgttcagtttggcgttactagcagtaatgggtatacaactagttgttactaccggaacgatccttgctgaaaatagtatcaaagtagattcaaatcttgcgttaacatcggatcaggaattatctaatgaaaaacaaattgtaattagagcgagcgatcagtcaaaagcgcaacgtgatttagcgctaactataccagagggggccacatttgatcgcacaactagtaatcaattaaacaaaaaatcagcgtatacggctacccttgatcaaaaaaataattccaatcaagttattttaaaacaaacagcacaaacagctgaaaatattgagaatgatttggtaaatagacaagcaacaacgttcaaaaaagaatacttattagtttttaaactaaaagatactggtatatccgctaaacaacagttgaaagtgacaacaataatgaacgggattcgctatgaatctaaaccactattgctgaaagaggtcgttggaacagatggtgttcaacgacaaactagtttgacgaaggagggtgaacctcaaataacctttaatctaaaccaaacagaatatatcgttggagataccatccagttaaaagttgatcttgatgctagcgaaattaatgaacctttctatattaatcttggcaataaagcaatactaacgccgataaaagtatctagcgataacaggaatattaactttgaatcacaaaataatcagaatacagggaagaacattttaaagatttccagtaacgaaccgataaagaaagattttacgctactagtaaaagcgacaacagttgggaaggtcgatattactgcttctaccgaaactgatcatttaatttcgaagacagctactgttaaagtggttgataacccaacatcaaaagtattagcaattaatcccaatacttttgatgacggtattcctaattgggttcaaaagacaaaaacagaggtcggaagtaagtcttttcaaagaagtacaagtcgatttcttaat

SEQ ID NO: 38 - DNA, 1020 bp, Lactobacillus sake
atgttcaggttgttgttgttagcagtaatgggtatacaactgattgttactaccggaacgagtcttgctgaaagtagtatcaaagtagattcagatcttgcattaacatcggatcaggaattatctaatgaaaaacaaattgtaattagggtgagcgatcagtcaaaagcgcaacgtgatttagggttaactataccagagggggccgcatttgatcgcacaactagtaatcaattaaacaaaaaatcagcgtatacggctatccttgatcaaaaaaataattccaatcaagttattttaaaacaaacagcacaaacagcacaaacagctgaaaatattgagaacgattcggtaaatagacaagcaacaacgttcaaaaaagaatacttattagtctttaaactaaaagatactagtatattcgctaaacaacagttgaaagtgacaacaataatgaacggcgttcactatgaatctaaaccactattgctgaaagatatcgttggaacagatggtgttcaacgacaaactagtttgtcgaaggaggatacacctcaaataacctttaatctaaatcaaacagaatatatcgttggagataccatccagttaaaagttgatcttaatgctagtgaaatcaatgagcctctctatattaatcttggcaataaagcaatactaacgccgataaaaatatctagcgataacgggggtattaactttgaatcacaaaataatcagaatacagggaagaacattttaaagatttccagtaacgaaccgataaagaaagattttacgctactagtaaaagcgacaacagttgggaaggtcgatattacggcttctaccgaaattgatcatttaatttcgaagacagctactgttaaagtgattgataacccaacatcaaaagcattagcaattaatcccaatacttttgatgacggtattcctaattgggttcaaaagacaaaaacagaggtcggaagtaaggcttttcaaggaagtacaagtcga

SEQ ID NO: 39 - DNA, 2040 bp, Lactobacillus sake
ccatggccaagttggtcgctaacatcattggtatcgtccaaaaaaactacggcattgtgctagacgaagagtcgttcaattacagtcgcttcatctcccatcttcgctacttcatcattcgacgtctcaaaaagacgcaagaaacagacacacaacttgatccggcactacttgctatgatgatcacacgttatccacaagcgtatcaagttgtccaacaaattacggcatttttgaaacttgagatagattgggccttacaatccgacgaagaagtttacttattattgcatatctggcgtgtcacccagcatcaacaaactgaataaaatggtgtgttactgattcgatcaggcattaacccagttaacgaatgcgacctatttttaggctgtattcattagctgggtttttgttttaacaattattaatctaaggaggtactatcatggcttacgaacaacttgcaacagacatcttggcaaacgtgggcggacccgaaaatatccgcagcgtcgtccactgtacgacgcgcttgcgcttcaaattgaaggacgaaaaaaaggcgcacactgatattttaaaaaatatggacggcgttgtgacggttgtccaaagtggtggtcaataccaagtcgttatcggtaatcaagtcgctgacgtttatgacgcacttttgaaaatcggcaatattaccggtgaaggcatcgtccctgatgatgacgaccaacaacctaaaatgagtttactcgataaattcatcgatttaatttctggcatcttcgcaccaacgcttggcgtcttagcggcaaccgggatgattaaagggctcgtcgccctctttgtcgcagtcggttggctcaccatcaaatctggcacttatcaaattctatttgcgctcggcgattcattcttctatttctttccaattatgttaggctacagtgccagcaagaaattcaaagtcaatccgttcctcggtatggtgattggggcctccctcgtttacccgtcaatcgtgggacgagtcccttcagcgctggcgcaaacaggcgctaaaccactttacactttgtttgccggtacgctattcgaatcaccaatttacacaacattccttggtattccagtgattatgatgagttacgcgtcatccgtcatcccaatcgtggttgctgtttggttcacctcaaaagttgaacccttcttcaaaaaagtcttacccgaagtgattcggacattcttagtaccgttttgtacggccctcgtcgttgtgccggtgaccttcttagtgattggtccactgtcaacttggcttggtgatctattaggcgctggtagtaatgcgctctatcaattcagtccagtagttactggggcagtgcttggtggcctctggcaagtgctcgttatcttcggccttcattgggggattgtcccactggcaattctcaacatttccacactgggttacgacacgattttaacacttgtctttgccgcatcctttgcacaaatcggatctgttttagccatcacattacgaacgaaaaataaaaagctcaaaggcttaggctggtcatctttcatctctgggattttcggtgtcacagaacccgtaatttacgggattacactgccacgtaaaaagccattcatcttcagttgtatcggtggtgcagtcggcggattgctactcggcttatttggcactcgcggctacctcgtgggtggactcggtgtcttcggtattccgaactttatcaatccaaagaccggtctcgatatgaccgtttacggtgcgctaattgctgccgctgttgccttcgtaattgcattattattaacacttttcttcggcatttctaacgaagatcaagtggcaaccgttgaacccgctgctcctatggcacctactcctccgacagaagcgcctgccaaagtaacacctgaaatcattactagtccccttagtggcaccgtaatacctctttcagaactaaaagatcctgttttctcatcagctgtcatgggcaaagg

SEQ ID NO: 40 - DNA, 5935 bp, Lactobacillus sake
tacaagcgcttgaatggattgtaattggtaacggtgtcggttttggcaaacaccctggcgacttggtcgatgaatcaaagattgaacgccgttttatcgcaaccggttcaaaggggttcgatgcaacccaggttcaggcactaacagaaatcaatgcaaaaatgctaaccattatgtccgaaatcgttacgttagttgagccagcattagatactaagattaacgatcgccaatatctgattctagcggaccatctccaattcgccgttaaacgcatcaacgcccatgtcacgttaaacgatcaatcattactctgggaattacgtaatttatttccaactgaatataaaactgcgcagcagactattcaactgatcaatcaaaaattaggcttcaaactacccgatggtgaagcagtgcctctcacttatcattttgtgaatatcaaaaatggcgacagtgaaatcgaagatacgatgaccatggccaagttggtcgctaacatcattagtatcgtccaaaagaactacggcattgtgttagatgaagagtcgttcaattacagtcgtttcatctcccatcttcgctacttcatcattcggcgtctcaaaaagacgcaagaaacagacacacaacttgaccccgcactgcttgccatgatgatcacacgttatccacaggcgtatcaagttgtccaacaaatcacggcctttttaaaacttgagatggattggaccttacaatctgacgaagaagtttacttattattgcatatctggcgtgtcacccagcatcaacaaactgaataaaatggtgtgttactgattcgatcaggcattaacccagttaacgaatgcgacctatttttaggctgtattcattagctgggtttttgttttaacaattattaatctaaggaggtactatcatggcttacgaacaacttgcaacagacatcttggcaaacgtgggcggacccgaaaatatccgcagcgtcgtccactgtacgacgcgcttgcgcttcaaattgaaggacgaaaaaaaggcgcacactgatattctaaaaaatatggacggcgttgtgacggttgtccaaagtggtggtcaataccaagtcgttatcggtaatcaagtcgctgacgtttatgacgcacttttgaaaatcggtaatatcgctggtgaaggcatcgtccctgatgatgacgaccaacaacctaagatgagtttactcgataaattcatcgatttaatttctggcatcttcgcaccaacccttggtgtcttagccgcaactgggatgattaaaggactcgtcgcgctctttgtcgtagtgggctggctcacgactaaatctggcacttatcaaattttattcgcactcggtgattcattcttctatttctttccaattatgttaggctacagcgctagcaagaagttcaaggtcaatccgtttctcggtatggcgattggcgcctccctcatttacccatcaatcgtgggcttagtcccttcagcgctggcgcaaactggcgctaaaccactttacactttgtttgccggtacgctatttgagtcaccaatctacacaacattcctcggtattccagtaattatgatgagttacgcgtcatccgtcatcccaatcgtggttgccgtttggttcacctcaaaagtcgaacccttcttcaaaaaagtcttacccgaagtgattcggacattcttagtaccgttttgtacggccctcgtcgttgtgccggtgaccttcttagtgattggtccactgtcaacttggcttggtgatctattaggcgctggtagtaatgcgctctatcaattcagtccagtagttactggggcagtgcttggtggcctctggcaagtgctcgttatcttcggccttcattgggggattgtcccactggcaattctcaatatttccacattaggttacgacacgatcttaacgcttgtctttgccgcatcctttgcacaaatcggatctgttttagccatcacattacgaacgaaaaataaaaagctcaaaggcttaggccggtcatccttcatctctgggatttttggtgtcacagaacccgcaatttacgggattacactgccgcgtaaaaagccattcatcttcagttgtatcggtggtgcagtcggtggattgctactcggcttattcggcactcgcggttacctcgtgggtggacttggtgtcttcggtattccgaactttatcaatccaaagaccggtctcgatatgaccgtttatggtgcgttaattgctgccactgttgccttcgtaattgcattattattaacacttttcttcggcatttctaaagaagatcaagtggcaacggttgaacccgcaactcctatgacaccaactccggcagaaacccctgccaaagtaacgcccgaaatcattactagtccccttagcggcaccctagcacctctttcagaacttaaagatcctgttttctcatcagcagtcatgggtaaaggagtggcaatcattccttctgatggccaacttttcgcacccgttgccggaacagtcagtctcgtctttccaactggacacgcaattggtattaattctgaacacggtgctgaaattttaatgcatgtcgggatggataccgtccaacttgagggcaaaccatttacggtacatgtgaaacagggtcaagcagtcctccccggtgatttattattaaccttcgatattgaagccattcaagcggccggttacgaaatcgcgacaccggtcatcattactaatagtaatttgtacgattcagtggcacctctgatttcaaccggaactgtgtctactggcgatcaactcttaactttaaaataacattcaaaggagactaaagatgactaatcacacccctttcccaaaagattttctctggggcggcgcaactgccgctaaccaatatgaaggtgcttaccaaaccgacggcaaaggtttgtcgctcgttgatattttgccagatgctcaccatggccgctgggaagccctaatgaatcctaaaaaagcgcaagcgactcaatacgacttctatccaagtcacaaatccgttgatttctatcatcattatgaagaagacctgcgcttgatggctgagatgggctttaaaacctatcggctatcaattagctggcctcgtattttcccaaatggtgatgatttaacacctaacgaagccggtctcgccttttacgatcgcgtgattgatctgtgtcgcgagctcaacattgaaccactcgtgactatcaaccatttcgatactccactagcgatgatggataagtgtaacggctgggccgatcgccggatgatcgaccactatctgcgcttttgttcggtgctttttaaacgctatagaggtaaagttcactattggttaacgtttaacgaaatcaacatgattctccacattccgttcttaggcggtggcatggacgtttccgataccgctcatccagatcaaattaaataccaagcttcccatcaccaattagtggcttcagcgttagcaaccaagcttgcacatgaaatcgatccagataatcaaattggctgcatgctagccgctggcgaaacttatccgaatacgccaaatccagcggatgtcttcaaagcacttgaaaccaatcgcgaaggctacttcttcatcgatgtgcaatcacgtagtgcttatccgaactatgccaagcggttatttaaagccaaaaatatcgtattagcaatgggacctgatgatgaggcgattctcaaagccaatacagttgacttcatttcatttagctattactcatctcgtttagttagtgctgatccagcggtgaatgaacaaactgaaggtaacgtcttcgcctcgcttaaaaatccatatctcaaaacaagcgaatggggctggcaaactgacggtcttggcttacggacgacaatgaatcaactctatgatcgatatcaaaaaccactgttcatcgttgaaaacggcctaggtgcacgtgatacagtcactgctgatggccaagttcacgacgactaccgtatcagttatctgcaagaaaatatcaccgctatgggccaagcaattgccgacggcgtaccttgtatcggttacacttcctggggctgtatcgatttagtcagcgccggttcaggtgaaatggacaagcgttacggttacgtatacgttgaccgcgacaatgatggtaacggcacattagcccgtacaccaaaggattctttctactggtatcaaaatgtcattaaaaccaatggtagcgtgttatataacggccaaacattttaatttttggagggacaattatgtattcaaaacaaccaattaccggctttaaaccagatttcttatggggtggtgcaaccgcggctaatcaaattgaaggtgcctggaatatcgacggcaaggggctcacgaccgccgaagtcgtccaaaaagcgaccgatcgccagaatttttcaatgaacaccgtgactaaagacagcattcaagtcgcaattgacgatccaaccgatacgctttatccaaaacggcgcggaatcgatttttatcaccactacaaagaagacatcgcattattcgccgaaatgggcttcaaggctttccgggtgagtatcgcgtgggctcgtatttttccaaacggcgatgacgcgcaccccaatgaaataggcttagccttctacgataacgtcttcgctgaactcaaaaaatacaatatcgagccagtcgtgaccctctcccattatgaaatgccgcttgcactgacactgaaatataacggttgggccagtcgcgaaaccatcactgcgtttaaccgttataccgaaacggtcttcaaacgttatcaacatcaagttaaatactggatgacttttaacgaaattaatgccgcaacttggggattcactggtaccggcgcggttgacgataccctctccccaaatgagcaattacaactgcgctatcaagcgctgcatcatcaatttgttgcgagcagtatcgccgttaaacaatgtcacgaattaatgccaaacgccaaaattggttcaatgttggcgcggatgcaaagttatcctaaaacatcgaacccagtcgatgtccgtcaagcgcaagaagatgatgaattgaatctctttttcacagatgttcaggtccgtggcgaatatcccaactatatgaaccgttacttcaacgaccacggcatcgaacttgaaatggcacctgacgacttacaattaatcaaagattatccggtcgattatttaagcttcagctattacatgtcaatggtcagctcagctagacccgccggtgaaaaaacagctggcaacttaattctcggtgaaaaaaatccgtatctcgaatcaagcgactggggttggcaaattgatcccgtcggtttacggattaccctcaagaatctctgggaacgctatggtgtaccactattcatcgtcgaaaatggcttaggcgcaatcgataaggttgcagctgacggtcaaattcatgacccttatcgtattgactacatgcgcaaacacatcgcacagatgaaagaggccgttcaagacggcgtggatctaatgggctacaccatgtggggcccaatcgacttaattagtgcctctacctctgaaatgtcaaaacgctacggcttcatctacgtcgaccaagatgatgacggtaacggtacgctcgaacggagccgtaaagactcgttctactggttcaaaaatgtcattgctagcaatggcgaagatctctagaacatagaaaaagcccccgacttttatcaggggctttttcttatactgtaaaaataaatcggtcgaatgcttgcgtaatcccaacataattgttatcggcagtaataa

SEQ ID NO: 41 - DNA, 930 bp, Lactobacillus sake
accacttataacgatgccttatattgcccgtgttttaggcccaacgggtgttgggattaactcatttaccaattcaaatacacagtattttattttaattggtagtatcggtgtttcattgtatggtaatcgccagattgcttattatcgagatgataaagcgaaaacttctcaaattttttgggaagtttttctaatgagaatgattacgattcttgtagcactagcagcattctttattttcttagggttcgagaaatcttgtcaccaagcatatctgatgcaagcaattttaattgttgctgcagcttttgatatttcatggttctttatggggtttgagaatttcaaggtaacggttcttagaaatattatcgttaagttaatatcgttagcttgtattttcatatttgttcgtgataaaggtgatttgacattgtacattgctgttttgtcaatttctcaattaatcggtaatattacgttatttccttatttaaaacgatacattaacttaccaaattggcgagaattaaaaatttggcgtcattttaaaccttctttagttttatttgttccccaaattgcaacccaattttacttgattcttaataaaacaatggttgggaagatggtatcagttgaagcagcaggcttctacgataattccgataaaattatcaagatggtattagcgattgtaaccgcgacaggaacagtgatgttacctcgagtcgctaatacatttgctaagggagatcatgaacaggttaaaaaatacttatatcaatcttttgattttgtgagcgccatttcgataccgatgatgtttgggatggcggccatcgctcctaaatttgctacgatgttttttggacaagcatttgatgcagtaggaccactaatgatggttgaatcgttagtaattat

SEQ ID NO: 42 - DNA, 21 bp, Artificial Sequence
  Probe
ggcccaagaagtgacggctgt

SEQ ID NO: 43 - DNA, 29 bp, Artificial Sequence
  Primer
taacttgggaactgcatttgaaactggtc

SEQ ID NO: 44 - DNA, 23 bp, Artificial Sequence
  Primer
cgccactggtgttcctccagatc

SEQ ID NO: 45 - DNA, 25 bp, Artificial Sequence
  Primer
attggcaattggcgctgaagaaggt

SEQ ID NO: 46 - DNA, 23 bp, Artificial Sequence
  Primer
gctcagccatatcagctgggatc

SEQ ID NO: 47 - DNA, 28 bp, Artificial Sequence
  Primer
acgaacggataaagagcttgctcttttg

SEQ ID NO: 48 - DNA, 29 bp, Artificial Sequence
  Primer
cgaaaccgtctttcacttgaacatcttat

SEQ ID NO: 49 - DNA, 28 bp, Artificial Sequence
  Primer
ggaccagaggttatcgaaacattaactg

SEQ ID NO: 50 - DNA, 24 bp, Artificial Sequence
  Primer
taataccagcagcaggaattgctt

SEQ ID NO: 51 - DNA, 28 bp, Artificial Sequence
  Primer
ttgctcagatatgacgatggacattgca

SEQ ID NO: 52 - DNA, 28 bp, Artificial Sequence
  Primer
gaccatctcttagtgatagcaagaccat

SEQ ID NO: 53 - DNA, 30 bp, Artificial Sequence
  Primer
ggtcttcggattgtaaagcactttaagttg

SEQ ID NO: 54 - DNA, 26 bp, Artificial Sequence
  Primer
gtagttagccggtgcttattctgtcg

SEQ ID NO: 55 - DNA, 22 bp, Artificial Sequence
  Primer
gtgaaagcccggagctcaactc

SEQ ID NO: 56 - DNA, 29 bp, Artificial Sequence
  Primer
ctggtgttcttccacatatctacgcattc

SEQ ID NO: 57 - DNA, 21 bp, Artificial Sequence
  Primer
ctggctatcccgatacatacc

SEQ ID NO: 58 - DNA, 20 bp, Artificial Sequence
  Primer
gcatatcttggctacgggca

SEQ ID NO: 59 - DNA, 25 bp, Artificial Sequence
  Primer
atctagtagactctgaatttaaggt

SEQ ID NO: 60 - DNA, 24 bp, Artificial Sequence
  Primer
ttaaccgcttctaacaccttttga

SEQ ID NO: 61 - DNA, 22 bp, Artificial Sequence
  Primer
ctctacctctgaaatgtccaag

SEQ ID NO: 62 - DNA, 23 bp, Artificial Sequence
  Primer
tttagaggtcttcaccattgttg

SEQ ID NO: 63 - DNA, 21 bp, Artificial Sequence
  Primer
atttattccagccagcgtttc

SEQ ID NO: 64 - DNA, 22 bp, Artificial Sequence
  Primer
tgtggaaaacattcatgtaccg

SEQ ID NO: 65 - DNA, 18 bp, Artificial Sequence
  Primer
aattgggtgcctgccgcg

SEQ ID NO: 66 - DNA, 20 bp, Artificial Sequence
  Primer
gaataagagcgcgaatacgg

(19) United States
(12) Patent Application Publication
(10) Pub. No.: US 2015/0272144 A1
(43) Pub. Date: Oct. 1, 2015

(54) COMPOSITION COMPRISING AT LEAST THREE DIFFERENT STRAINS OF L. SAKEI FOR PRESERVING EDIBLE PRODUCTS

(71) Applicant: INSTITUT NATIONAL DE LA RECHERCHE AGRONOMIQUE, Paris (FR)

(72) Inventors: Stephane Chaillou, Orgerus (FR); Marie-Christine Champomier-Verges, Sceaux (FR); Monique Zagorec, Nantes (FR)

(21) Appl. No.: 14/421,046

(22) PCT Filed: Aug. 13, 2013
(86) PCT No.: PCT/EP2013/066886

§ 371 Date: Feb. 11, 2015

(30) Foreign Application Priority Data
Aug. 13, 2012 (FR) 1257785

(51) Int. Cl. A23B 4/22; C12N 1/20; A23L 1/314

(52) U.S. Cl. CPC A23B 4/22; A23L 1/31472; C12N 1/20; A23V 2002/00; A23Y 2220/77

(57) ABSTRACT

The present invention relates to a composition comprising at least three different strains of L. sakei bacteria, and to the use thereof for preserving edible products.

The present invention relates to a composition comprising at least three different strains of Lactobacillus sakei (L. sakei) bacteria, and its use for preserving edible products, preferably meat products, in particular beef meat and even more particularly beef carpaccio.

Meat products are food which, without preservation treatment, is quickly spoiled, at a rate which depends on various factors: product acidity, ambient moisture content, modified atmosphere, type and load of bacteria, temperature . . . .

Numerous treatments for preserving food, and in particular meat products, are known (fermentation, smoking, salting, drying, addition of preservatives, vacuum packaging . . . ). However, the use of preservatives is regulated. Moreover, most of these treatments cause a transformation of the fresh product (taste, color, texture). It is also known to use protective cultures for bio-preservation purposes, the intended effect being then to insure the microbiological quality without organoleptic change of the product, so as to preserve its fresh character. This implies therefore the use of bacteria, harmless to human health, which will prevent other undesirable bacterial species from being developed, without modifying the food base. Research for engineering good protective cultures has been developing for several years, and the major difficulty lays in the fact that the competition between different bacterial species on complex food substrates results from a set of mechanisms still poorly understood (Devlieghere et al., 2004). Some examples exist (in France: lactic ferment LLO for prawn preservation; in the USA: BovamineMeat™ for meat preservation; in Europe Holdbac™ and SafePro Ranger™ marketed by Danisco and Ch Hansen for meat products preservation). However, existing products only target a single bacterial species (Listeria monocytogenes) and are not concerned with beef carpaccio.

More particularly, as regards meat products, the meat intended to be consumed raw or undercooked (steaks tartare or beef burger, carpaccio for example) or ready-cooked food, are those that can have some significant contamination problems with a repercussion on the consumer health. Indeed, in the case of products consumed raw or undercooked, pathogenic bacteria are not destroyed during cooking, and in the case of ready-cooked food, made sterile by prior cooking, a subsequent contamination can be hazardous because in the absence of competing species, newly contaminating bacteria can develop up to a high degree. This is thus substantially on these types of products that research on protective cultures of meat products has been developed (Bredholt et al., 2001; Vermeiren et al., 2004; Vold et al., 2000). It has been observed that the endogenous flora of meat, and in particular L. sakei, contributes to preventing pathogenic bacteria such as Escherichia coli O157:H7 from being developed in chopped meat. Tests made using anti-Listeria bacteriocin producing strains have shown that they limited the development of Listerias on cooked meat products. However, the effectiveness of bactericins in food bases remains poorly controlled and the range of targets is restricted to a few bacterial species.

There is a very wide diversity of strains of L. sakei the size of the genome of which ranges from 1.8 Mb to 2.3 Mb. This wide diversity is divided into around ten genetic classes within the species. Thus, the inventors have identified different genetic markers enabling the strains of L. sakei to be characterized (international applications WO 2009/106491 and WO 2011/023675).

In spite of the knowledge of this diversity, it is currently impossible to determine whether there is an “ideal” assembly of strains which could fight most efficiently against the alteration of one or more altering species in a meat product, in particular of the beef carpaccio type.

Surprisingly, the inventors have engineered a specific composition comprising 3 classes of L. sakei, well identified and traceable in the product, which allows to fight against several food-altering bacterial species, in particular for meat products, more particularly raw or undercooked meat products and most particularly beef carpaccio and beef burger. These different classes of L. sakei are identifiable by specific markers.

Thus, the present invention relates to a composition comprising at least three different strains of L. sakei bacteria, wherein:
said first strain of L. sakei bacteria comprises in its genome the following sequence:
SEQ ID NO: 35 or a sequence exhibiting at least 95% identity thereto,
said second strain of L. sakei bacteria comprises in its genome the following sequence:
SEQ ID NO: 21 or a sequence exhibiting at least 95% identity thereto,
said third strain of L. sakei bacteria comprises in its genome the following sequence:
SEQ ID NO: 26 or a sequence exhibiting at least 95% identity thereto.

Preferably, said first strain of L. sakei bacteria comprises in its genome the following sequences:
SEQ ID NO: 42, and
SEQ ID NO: 35 or a sequence exhibiting at least 95% identity thereto,
preferably said first strain of L. sakei bacteria comprises in its genome the following sequences:
SEQ ID NO: 2 or a sequence exhibiting at least 95% identity thereto,
SEQ ID NO: 34 or a sequence exhibiting at least 95% identity thereto,
SEQ ID NO: 42, and
SEQ ID NO: 35 or a sequence exhibiting at least 95% identity thereto;

preferably said first strain of L. sakei bacteria comprises in its genome the following sequences:
SEQ ID NO: 2 or a sequence exhibiting at least 95% identity thereto,
SEQ ID NO: 4 or a sequence exhibiting at least 95% identity thereto,
SEQ ID NO: 6 or a sequence exhibiting at least 95% identity thereto,
SEQ ID NO: 7 or a sequence exhibiting at least 95% identity thereto,
SEQ ID NO: 8 or a sequence exhibiting at least 95% identity thereto,
SEQ ID NO: 9 or a sequence exhibiting at least 95% identity thereto,
SEQ ID NO: 10 or a sequence exhibiting at least 95% identity thereto,
SEQ ID NO: 13 or a sequence exhibiting at least 95% identity thereto,
SEQ ID NO: 15 or a sequence exhibiting at least 95% identity thereto,
SEQ ID NO: 19 or a sequence exhibiting at least 95% identity thereto,
SEQ ID NO: 32 or a sequence exhibiting at least 95% identity thereto,
SEQ ID NO: 34 or a sequence exhibiting at least 95% identity thereto,
SEQ ID NO: 42,
SEQ ID NO: 35 or a sequence exhibiting at least 95% identity thereto,
SEQ ID NO: 36 or a sequence exhibiting at least 95% identity thereto, and
SEQ ID NO: 38 or a sequence exhibiting at least 95% identity thereto, and

particularly preferably, said strain is the strain deposited with the CNCM (Collection Nationale de Cultures de Microorganismes; Institut Pasteur, 28 rue du Docteur Roux, F-75724 Paris CEDEX 15) on Nov. 19, 2010 as CNCM I-4396.

Preferably, said second strain of L. sakei bacteria comprises in its genome the following sequences:
SEQ ID NO: 21 or a sequence exhibiting at least 95% identity thereto, and
SEQ ID NO: 40 or a sequence exhibiting at least 95% identity thereto;

preferably said second strain of L. sakei bacteria comprises in its genome the following sequences:
SEQ ID NO: 16 or a sequence exhibiting at least 95% identity thereto,
SEQ ID NO: 17 or a sequence exhibiting at least 95% identity thereto,
SEQ ID NO: 21 or a sequence exhibiting at least 95% identity thereto, and
SEQ ID NO: 40 or a sequence exhibiting at least 95% identity thereto;

preferably said second strain of L. sakei bacteria comprises in its genome the following sequences:
SEQ ID NO: 4 or a sequence exhibiting at least 95% identity thereto,
SEQ ID NO: 8 or a sequence exhibiting at least 95% identity thereto,
SEQ ID NO: 9 or a sequence exhibiting at least 95% identity thereto,
SEQ ID NO: 11 or a sequence exhibiting at least 95% identity thereto,
SEQ ID NO: 12 or a sequence exhibiting at least 95% identity thereto,
SEQ ID NO: 16 or a sequence exhibiting at least 95% identity thereto,
SEQ ID NO: 17 or a sequence exhibiting at least 95% identity thereto,
SEQ ID NO: 20 or a sequence exhibiting at least 95% identity thereto,
SEQ ID NO: 21 or a sequence exhibiting at least 95% identity thereto,
SEQ ID NO: 36 or a sequence exhibiting at least 95% identity thereto,
SEQ ID NO: 38 or a sequence exhibiting at least 95% identity thereto,
SEQ ID NO: 40 or a sequence exhibiting at least 95% identity thereto,

preferably said strain further comprises one, preferably, 2, 3, 4, 5, 6, 7, 8, 9, 10, 11, 12, 13 and even preferably 7, 8, 9, 10, 11, 12 of the sequences selected from the group consisting of the sequences: SEQ ID NO: 5, SEQ ID NO: 10, SEQ ID NO: 14, SEQ ID NO: 15, SEQ ID NO: 18, SEQ ID NO: 19, SEQ ID NO: 24, SEQ ID NO: 29, SEQ ID NO: 31, SEQ ID NO: 32, SEQ ID NO: 33, SEQ ID NO: 37, and SEQ ID NO: 41, and/or a sequence exhibiting at least 95% identity thereto respectively,

and particularly preferably, said strain is the strain deposited with the CNCM on Nov. 19, 2010 as CNCM I-4393.

Preferably, said third strain of L. sakei bacteria comprises in its genome the following sequences:
SEQ ID NO: 23 or a sequence exhibiting at least 95% identity thereto, and
SEQ ID NO: 26 or a sequence exhibiting at least 95% identity thereto,

preferably said third strain of L. sakei bacteria comprises in its genome at least the following sequences:
SEQ ID NO: 22 or a sequence exhibiting at least 95% identity thereto,
SEQ ID NO: 23 or a sequence exhibiting at least 95% identity thereto, and
SEQ ID NO: 26 or a sequence exhibiting at least 95% identity thereto,

preferably said third strain of L. sakei bacteria comprises in its genome at least the following sequences:
SEQ ID NO: 7 or a sequence exhibiting at least 95% identity thereto,
SEQ ID NO: 13 or a sequence exhibiting at least 95% identity thereto,
SEQ ID NO: 15 or a sequence exhibiting at least 95% identity thereto,
SEQ ID NO: 20 or a sequence exhibiting at least 95% identity thereto,
SEQ ID NO: 22 or a sequence exhibiting at least 95% identity thereto,
SEQ ID NO: 23 or a sequence exhibiting at least 95% identity thereto, SEQ ID NO: 26 or a sequence exhibiting at least 95% identity thereto,

and preferably said strain further comprises one, preferably 2, 3, 4, 5, 6, 7, 8, 9, 10, 11, 12, 13, 14 or 15 and even preferably 5, 6, 7, 8, 9 of the sequences selected from the group consisting of the sequences: SEQ ID NO: 3, SEQ ID NO: 5, SEQ ID NO: 9, SEQ ID NO: 12, SEQ ID NO: 14, SEQ ID NO: 25, SEQ ID NO: 27, SEQ ID NO: 28, SEQ ID NO: 29, SEQ ID NO: 30, SEQ ID NO: 31, SEQ ID NO: 36, SEQ ID NO: 37, SEQ ID NO: 38, and SEQ ID NO: 41 or a sequence exhibiting at least 95% identity thereto respectively,

and particularly preferably, said strain is the strain deposited with the CNCM on Nov. 19, 2010, as CNCM I-4394.

Thus, preferably, the composition according to the present invention comprises:
a first strain of L. sakei bacteria comprising in its genome the following sequences:
SEQ ID NO: 42, and
SEQ ID NO: 35 or a sequence exhibiting at least 95% identity thereto,
a second strain of L. sakei bacteria comprising in its genome the following sequences:
SEQ ID NO: 17 or a sequence exhibiting at least 95% identity thereto, and
SEQ ID NO: 40 or a sequence exhibiting at least 95% identity thereto;
a third strain of L. sakei bacteria comprising in its genome the following sequences:
SEQ ID NO: 23 or a sequence exhibiting at least 95% identity thereto, and
SEQ ID NO: 26 or a sequence exhibiting at least 95% identity thereto.

Particularly preferably, the composition according to the present invention comprises:
a first strain of L. sakei bacteria comprising in its genome the following sequences:
SEQ ID NO: 2 or a sequence exhibiting at least 95% identity thereto,
SEQ ID NO: 34 or a sequence exhibiting at least 95% identity thereto,
SEQ ID NO: 42, and
SEQ ID NO: 35 or a sequence exhibiting at least 95% identity thereto,
a second strain of L. sakei bacteria comprising in its genome the following sequences:
SEQ ID NO: 16 or a sequence exhibiting at least 95% identity thereto,
SEQ ID NO: 17 or a sequence exhibiting at least 95% identity thereto,
SEQ ID NO: 21 or a sequence exhibiting at least 95% identity thereto, and
SEQ ID NO: 40 or a sequence exhibiting at least 95% identity thereto; and
a third strain of L. sakei bacteria comprising in its genome the following sequences:
SEQ ID NO: 22 or a sequence exhibiting at least 95% identity thereto,
SEQ ID NO: 23 or a sequence exhibiting at least 95% identity thereto, and
SEQ ID NO: 26 or a sequence exhibiting at least 95% identity thereto.

Particularly preferably, the composition according to the present invention comprises:
a first strain of L. sakei bacteria comprising in its genome the following sequences:
SEQ ID NO: 2 or a sequence exhibiting at least 95% identity thereto,
SEQ ID NO: 4 or a sequence exhibiting at least 95% identity thereto,
SEQ ID NO: 6 or a sequence exhibiting at least 95% identity thereto,
SEQ ID NO: 7 or a sequence exhibiting at least 95% identity thereto,
SEQ ID NO: 8 or a sequence exhibiting at least 95% identity thereto,
SEQ ID NO: 9 or a sequence exhibiting at least 95% identity thereto,
SEQ ID NO: 10 or a sequence exhibiting at least 95% identity thereto,
SEQ ID NO: 13 or a sequence exhibiting at least 95% identity thereto,
SEQ ID NO: 15 or a sequence exhibiting at least 95% identity thereto,
SEQ ID NO: 19 or a sequence exhibiting at least 95% identity thereto,
SEQ ID NO: 32 or a sequence exhibiting at least 95% identity thereto,
SEQ ID NO: 34 or a sequence exhibiting at least 95% identity thereto,
SEQ ID NO: 42,
SEQ ID NO: 35 or a sequence exhibiting at least 95% identity thereto,
SEQ ID NO: 36 or a sequence exhibiting at least 95% identity thereto,
SEQ ID NO: 38 or a sequence exhibiting at least 95% identity thereto, and
a second strain of L. sakei bacteria comprising in its genome the following sequences:
SEQ ID NO: 4 or a sequence exhibiting at least 95% identity thereto,
SEQ ID NO: 8 or a sequence exhibiting at least 95% identity thereto,
SEQ ID NO: 9 or a sequence exhibiting at least 95% identity thereto,
SEQ ID NO: 11 or a sequence exhibiting at least 95% identity thereto,
SEQ ID NO: 12 or a sequence exhibiting at least 95% identity thereto,
SEQ ID NO: 16 or a sequence exhibiting at least 95% identity thereto,
SEQ ID NO: 17 or a sequence exhibiting at least 95% identity thereto,
SEQ ID NO: 20 or a sequence exhibiting at least 95% identity thereto,
SEQ ID NO: 21 or a sequence exhibiting at least 95% identity thereto,
SEQ ID NO: 36 or a sequence exhibiting at least 95% identity thereto,
SEQ ID NO: 38 or a sequence exhibiting at least 95% identity thereto,
SEQ ID NO: 40 or a sequence exhibiting at least 95% identity thereto,

preferably, said strain further comprising one, preferably 2, 3, 4, 5, 6, 7, 8, 9, 10, 11, 12, 13, 14, 15, 16 and even preferably 7, 8, 9, 10, 11, 12 of the sequences selected from the group consisting of the sequences: SEQ ID NO: 5, SEQ ID NO: 10, SEQ ID NO: 14, SEQ ID NO: 15, SEQ ID NO: 18, SEQ ID NO: 19, SEQ ID NO: 24, SEQ ID NO: 29, SEQ ID NO: 31, SEQ ID NO: 32, SEQ ID NO: 33, SEQ ID NO: 37, SEQ ID NO: 41, and/or a sequence exhibiting at least 95% identity thereto respectively, and
a third strain of L. sakei bacteria comprising in its genome at least the following sequences:
SEQ ID NO: 22 or a sequence exhibiting at least 95% identity thereto,
SEQ ID NO: 23 or a sequence exhibiting at least 95% identity thereto, and
SEQ ID NO: 26 or a sequence exhibiting at least 95% identity thereto,

preferably said third strain of L. sakei bacteria comprising in its genome at least the following sequences:
SEQ ID NO: 7 or a sequence exhibiting at least 95% identity thereto,
SEQ ID NO: 13 or a sequence exhibiting at least 95% identity thereto,
SEQ ID NO: 15 or a sequence exhibiting at least 95% identity thereto,
SEQ ID NO: 20 or a sequence exhibiting at least 95% identity thereto,
SEQ ID NO: 22 or a sequence exhibiting at least 95% identity thereto,
SEQ ID NO: 23 or a sequence exhibiting at least 95% identity thereto,
SEQ ID NO: 26 or a sequence exhibiting at least 95% identity thereto,

and preferably said strain further comprises one, preferably 2, 3, 4, 5, 6, 7, 8, 9, 10, 11, 12, 13, 14, 15, 16, 17 or 18 and even preferably 5, 6, 7, 8, 9 of the sequences selected from the group consisting of the sequences: SEQ ID NO: 3, SEQ ID NO: 5, SEQ ID NO: 9, SEQ ID NO: 12, SEQ ID NO: 14, SEQ ID NO: 25, SEQ ID NO: 27, SEQ ID NO: 28, SEQ ID NO: 29, SEQ ID NO: 30, SEQ ID NO: 31, SEQ ID NO: 36, SEQ ID NO: 37, SEQ ID NO: 38, and SEQ ID NO: 41 or a sequence exhibiting at least 95% identity thereto respectively.

Preferably, the present invention relates to a composition as defined above comprising at least one fourth strain of L. sakei bacteria, different from the other strains defined above.

Preferably, said fourth strain of L. sakei bacteria comprises in its genome at least the sequence SEQ ID NO: 39 or a sequence exhibiting at least 95% identity thereto.

In a preferred embodiment, the fourth strain comprises in its genome at least the following sequences:
SEQ ID NO: 4 or a sequence exhibiting at least 95% identity thereto,
SEQ ID NO: 6 or a sequence exhibiting at least 95% identity thereto,
SEQ ID NO: 7 or a sequence exhibiting at least 95% identity thereto,
SEQ ID NO: 8 or a sequence exhibiting at least 95% identity thereto,
SEQ ID NO: 9 or a sequence exhibiting at least 95% identity thereto,
SEQ ID NO: 15 or a sequence exhibiting at least 95% identity thereto,
SEQ ID NO: 31 or a sequence exhibiting at least 95% identity thereto,
SEQ ID NO: 36 or a sequence exhibiting at least 95% identity thereto,
SEQ ID NO: 39 or a sequence exhibiting at least 95% identity thereto,

and preferably said strain further comprises one, preferably, 1, 2, 3, 4, 5, and even preferably 1 or 2 of the sequences selected from the group consisting of the sequences: SEQ ID NO: 10, SEQ ID NO: 12, SEQ ID NO: 19, SEQ ID NO: 32 and SEQ ID NO: 38 or a sequence exhibiting at least 95% identity thereto respectively, and particularly preferably, said strain is the strain deposited with the CNCM on Nov. 19, 2010 as CNCM I-4395.

The genome of the 4 strains of L. sakei deposited with the CNCM as the following numbers: CNCM I-4393, CNCM I-4394, CNCM I-4395 and CNCM I-4396 has been sequenced by the inventors.

Preferably, said composition is a food composition. For the purposes of the present invention, by “food composition”, it is intended a composition likely to be integrated in a food product, that is a composition suitable for consumption. By “suitable for consumption”, it is meant a product assimilable by an individual, preferably a human, safely, that is not likely to generate a pathology in the short term.

Preferably, said composition is formulated so as to guarantee the viability of said at least three strains, preferably of said at least four strains.

Preferably, for the purposes of the present invention, the nucleotide sequences used as markers of the strains of L. sakei according to the present invention correspond to a nucleotide sequence exhibiting at least 95%, 96%, 97%, 98%, 99% identity with the sequences defined herein above.

By nucleotide sequence exhibiting at least 95% identity, preferably at least 96%, 97%, 98%, 99% identity to a sequence, it is intended the polynucleotides exhibiting, with respect to the reference polynucleotide, some modifications such as in particular a deletion, truncation, elongation, chimeric fusion and/or substitution, in particular a point one. These are preferably sequences coding the same amino acid sequences as the reference sequence, this related to the degeneracy of the genetic code, or complementary sequences which are likely to specifically hybridize to the reference nucleotide sequences, preferably under high stringency conditions.

A hybridization under high stringency conditions means that the temperature and ionic strength conditions are selected such that they enable the hybridization to be held between two fragments with complementary DNAs.

The percent identities referred to within the scope of the disclosure of the present invention are determined based on an overall alignment of sequences to be compared, that is an alignment of the sequences taken in their entirety, using for example the NEEDLEMAN and WUNSCH algorithm (J. Mol. Biol. 48, 443-453, 1970). This sequence comparison can be performed for example with the needle software using the parameter “Gap open” equal to 10.0, the parameter “Gap Extend” equal to 0.5, and a matrix “Blosum 62”. Needle software is for example available on the web site www.ebi.ac.uk, under the name “Align”.

Particularly preferably, the composition as previously defined comprises at least one, preferably two, preferably three, and even preferably four strains of L. sakei selected from the group consisting of: the strains deposited with the CNCM on Nov. 19, 2010 as the following numbers: CNCM I-4393, CNCM I-4394, CNCM I-4395 and CNCM I-4396.

Thus, preferably, the composition as previously defined comprises at least the 4 strains of L. sakei deposited with the CNCM as the following numbers: CNCM I-4393, CNCM I-4394, CNCM I-4395 and CNCM I-4396.

In a particular embodiment, the composition as previously defined does not comprise strains of L. sakei other than the four strains as defined above.

Preferably, the composition according to the present invention can comprise other preservative agents selected from the group comprising: pickles, such as red wine or soya based pickles, essential oils and bacteriocins.

Preferably, the composition according to the present invention comprises the at least four strains at equivalent concentrations.

Preferably, said strains are present at concentrations enabling the target flora to be desirably reduced in the edible product. Preferably, each of said strains is present in the composition at a concentration between about 10³ and 10⁸ cfu/mL, preferably at a concentration between about 10⁴ and 10⁷ cfu/mL, particularly preferably at a concentration between 10⁵ and 10⁶ cfu/mL.

Another aspect of the present invention relates to a method for preparing the composition according to the present invention comprising a step of culturing at least three strains, preferably at least four strains of L. sakei such as defined herein before in a food composition.

Preferably, said at least three strains, preferably at least four strains of L. sakei are introduced at equivalent concentrations.

Preferably, the at least three strains, preferably at least four strains of L. sakei are introduced at a concentration between 10³ and 10⁸ cfu/mL of food composition, preferably at a concentration between 10⁴ and 10⁷ cfu/mL of food composition, particularly preferably at a concentration between 10⁵ and 10⁶ cfu/mL of food composition.

Another aspect of the present invention relates to the use of a composition as defined herein before for preserving an edible product, preferably a meat product, in particular beef meat and even more particularly beef carpaccio or chopped beef and particularly preferably beef carpaccio.

Preferably, the meat product is raw or undercooked.

The chopped beef can for example be intended to be consumed as a beef tartare.

The composition according to the present invention enables the edible product to be preserved by virtue of a growth inhibition of some microorganisms likely to generate an alteration of said edible product, in particular during product storage.

The measurement of the alteration can be evaluated by the difference in the concentration of said microorganisms between the storage initial and final times, and can then be expressed as Δ Log₁₀ CFU/g of edible product between the final time and initial time.

The alteration of said edible product can in some cases generate a pathological response in an individual.

By way of example of microorganisms likely to generate an alteration of the edible product, there can be mentioned for example Enterobacteria from the group Serratia (Serratia, Hafnia, Obesumbacterium, Rahnella), Leuconostocs from the group mesenteroides (mesenteroides-carnosum-gasicomitatum-gelidum), Pseudomonas selected from the species P. fragi, P. putida and P. fluorescens, and some species, more specifically as Hafnia alvei, Brochothrix thermosphacta and Weisseila viridescens . . . .

Alteration is a phenomenon related to the growth of altering species and the amount of population reached by these species during storage.

For the purposes of the present invention, by “carpaccio”, it is intended thin slices, preferably with a thickness between 0.1 mm and 1 mm, of a raw edible product.

Another aspect of the present invention relates to a method for preserving an edible product as previously defined comprising a step of contacting said edible product with a composition as previously defined.

Preferably, said contacting step is performed before the step of packaging said edible product.

Preferably, said contacting step is performed by dipping said edible product into a composition as previously defined.

Preferably, said edible product is dipped into the composition as previously defined for 1 to 30 seconds, preferably 2 to 10 seconds, even preferably 4 to 6 seconds and particularly preferably for 5 seconds.

Alternatively, the contacting step is performed by spraying the composition according to the present invention onto said edible product.

For the purposes of the present invention, by “spraying”, it is meant atomizing said composition onto said edible product.

Alternatively, the contacting step is performed by blending the composition according to the present invention, preferably in a lyophilized form, with said edible product.

Preferably, the contacting step performed by blending the composition according to the present invention, preferably in lyophilized form, with said edible product is implemented when said edible product is beef burger.

Preferably, the contacting step is performed after the step of slicing or chopping the edible product, preferably meat.

Particularly preferably, the contacting step is performed for less than 1 hour, preferably less than 30 minutes, less than 15 minutes, less than 10 minutes, less than 5 minutes and preferably less than 1 minute after the step of slicing or chopping the edible product, preferably meat.

Preferably, the contacting step is followed by a step of vacuumizing or placing under protective atmosphere (such as an atmosphere comprising nitrogen, carbon dioxide and oxygen) of said edible product.

Particularly preferably, the vacuumizing step is performed for less than 1 hour, preferably less than 30 minutes, less than 15 minutes, less than 10 minutes, less than 5 minutes and preferably less than 1 minute after the contacting step.

Finally, the present invention relates to an edible product comprising a composition as previously defined.

Preferably, said edible product is selected from meat products, in particular a raw or undercooked meat product.

Preferably, said meat product, in particular said raw or undercooked meat product is beef meat, more particularly beef carpaccio or chopped beef and particularly preferably beef carpaccio.

The chopped beef can for example be intended to be consumed as a beef tartare.

Preferably, each of said strains is present in said edible product at a concentration between 10² and 10⁷ cfu/g of edible product, preferably at a concentration between 10³ and 10⁶ cfu/g of edible product, particularly preferably at a concentration between 10⁴ and 10⁵ cfu/g of edible product.

Preferably, said edible product comprises a total concentration of strains of the composition between 10³ and 10⁹ cfu/g of edible product, preferably between 10³ and 10⁴ cfu/g and 10⁸ cfu/g of edible product after contacting with the composition according to the present invention.

Preferably, said edible product comprises a total concentration of strains of the composition between 10⁴ cfu/g and 10⁸ cfu/g of edible product, preferably between 10⁵ cfu/g and 10⁷ cfu/g of edible product at the recommended last consumption date.

### DESCRIPTION OF THE FIGURES

FIG. 1 represents an example of Ct/CFU correspondence where it is observed that the q-PCR data are linear and correlated with the amount of bacteria in a range from 10² to 10⁷ CFU/g of carpaccio. The figure also shows an example where according to the primer used, either the species Hafnia alvei, either more generally the bacterial group (Serratia) to which H. alvei is related can be quantified.

Each circle corresponds to an experimental datum, the data giving a straight line. The accuracy of 0.1 Log 10 and the error margin of 0.3 Log 10 of data obtained by q-PCR are wholly smaller than those known for bacterial counts made on culture media.

FIG. 2 shows the result of quantifications of different species (genera/groups). Each circle shows the growth in Δ Log₁₀ (Log₁₀ at T_{14 days} after storage under vacuum and at 8° C., including a shock at 22° C. for 24 hours on day 7-Log₁₀ at T₀) of different species/groups/genera. Each circle corresponds to an experience (that is a batch of carpaccio from the market). (SER: Serratia group, HAL: Hafnia alvei species, BTH: Brochothrix thermosphacta species, WVI: Weisella viridescens species, LME: Leuconostoc genus (4 species), PSD: Pseudomonas genus (3 species), LSA: Lactobacillus sakei species). A great variability is observed from one batch to the other: the Serratia group for example can develop, during 14 days, from about 1.5 to about 4 Log₁₀ for example with a median value of 2.69 Log₁₀.

FIG. 3 shows the effects of different components of the composition on the growth of the target species. The measurement of these effects is performed by comparing cumulative growth equivalent (CGE) of different target species (five different cumulative measurements).

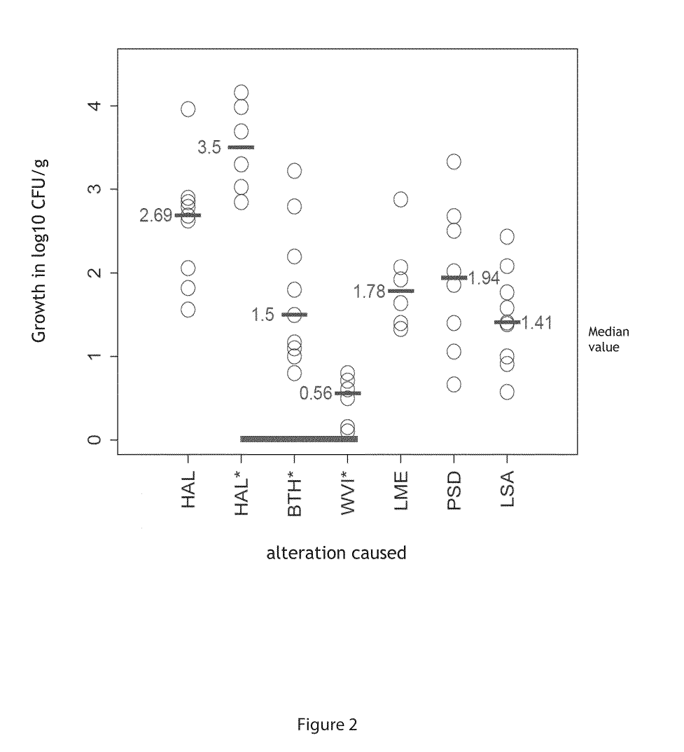

This CGE value is schematized as circles of different colors depending on the type of comparison performed (the scale is expressed in ΣΔ Log 10 CFU/g of meat and is measured at T14 days for the different samples (see storage conditions description in the caption of FIG. 2). The median value obtained from different batches (ΣΔ Log 10 CFU/g) is indicated by a figure adjacent to a horizontal dash. The values of the control N^{o} 1 (6 batches without adding composition) can be compared with those obtained with the same batches treated with the 4-strain composition of the invention (black circles) and with the 3-strain compositions of the invention (grey circles, 3 batches only). The codes of the compositions of the invention with 3 strains are: A=strains CNCM I-4395, CNCM I-4393, CNCM I-4394; B=strains CNCM I-4396, CNCM I-4393, CNCM I-4394; C=strains CNCM I-4396, CNCM I-4395, CNCM I-4393 and D=strains CNCM I-4396, CNCM I-4395, CNCM I-4394.

The values of control N^{o} 2 (6 other batches without adding a composition) can be compared with those obtained with the same batches treated with other 4-strain compositions used during the screening process, that is 3 series (noted PPB1 to PPB3) of 7 compositions.

FIG. 4 schematizes the quantification at T14 days of the strains of the composition of the invention by q-PCR on the DNA extracted from carpaccios using primers specific to five markers. The marker katA is specific to the L. sakei species (present in all the strains, that is those of the composition but also those of the L. sakei flora naturally present on the meat). The markers SEQ ID NO: 35, SEQ ID NO: 39, SEQ ID NO: 21 and SEQ ID NO: 26 are each specific of a single of the 4 strains of the composition.

FIGS. 5 to 7 relate to the definition of the classes of strains relative to the strains of the composition according to the present invention and as a function of the molecular imprinting.

FIG. 6 defines classes of strains relative to the strains of the composition of the invention and as a function of the 52-marker molecular imprinting of 4 other close strains.

FIGS. 6 and 7 show a comparison of molecular imprints between the 4-strain composition of the invention and the 21 other 4-strain compositions with 4 or 7 specific markers.

### EXAMPLES

### 1) Protocol of Forced Alteration of Beef Carpaccio

The experiments have been made on carpaccio purchased in the market, thus naturally contaminated.

a. Quantization of Altering Bacteria Naturally Present in Carpaccio

Some genera or species of altering bacteria were systematically present and readily quantifiable. They have thus been quantified without requiring a further addition to carpaccio (Enterobacteria of the Serratia group (including Hafnia alvei) (SER), Leuconostocs (mesenteroides-carnosum-gasicomitatum-gelidum) and Pseudomonas (fragi-putida-fluorescens) (PSD)).

b. Quantization of Altering Bacteria after Intentional Seeding of the Carpaccio

On the contrary, other genera or species of altering bacteria could be present but more hardly quantifiable, or poorly developing, or at very variable rates. These have been quantified both as natural and after an intentional seeding (Hafnia alvei, strain CIP57.31 (HAL) Brochothrix thermosphacta, strain INRA 160*7 (BTH), Weisseila viridescens, strain CIP102810 (WVI)) contaminants.

Therefore, an intentional inoculation protocol has been engineered where a bacterial suspension of a known load (in CFU/mL of suspension) was used to seed the carpaccio by dipping/dripping and then the load of carpaccio was measured (in CFU/g of carpaccio). Then, a chart has been used where the initial concentration of the bacterial suspension used to dip the carpaccios was sufficient to deduce the load of intentionally inoculated carpaccio.

An alteration protocol has been engineered which allowed a measurable development of different species: after inoculation by the compositions to be tested, the samples have been vacuum packaged, and then placed 7 days at 8° C., 24 hours at 22° C. and finally 6 days at 8° C.

### 2) Quantitative Measurements of Different Bacterial Genera/Groups/Species

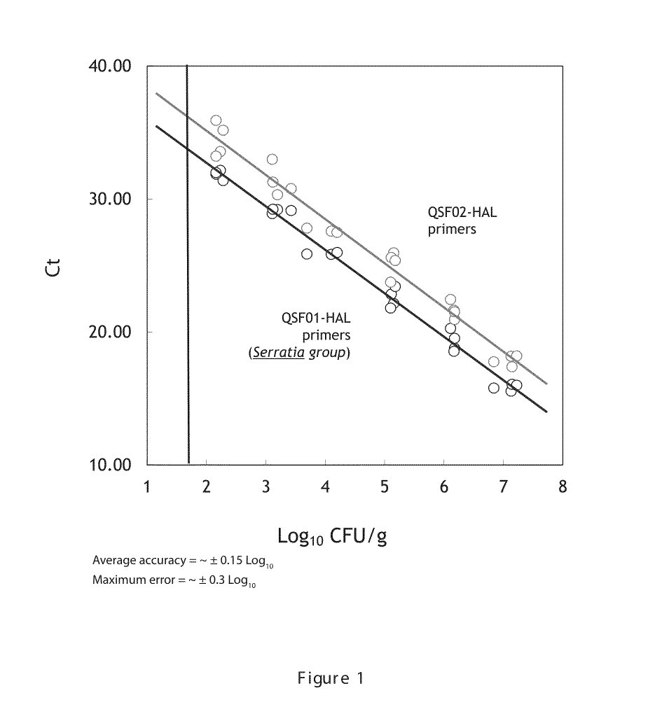

The quantitative measurements of different bacterial genera/groups/species are generally performed by spreading on more or less selective culture media. A protocol to set these measurements by quantitative real time PCR (q-PCR) has been developed. It required engineering (including the specificity and effectiveness test) primers which enable bacterial species/genera/groups to be measured from the bacterial DNA extracted from the carpaccios. Once the effectiveness and specificity of the primers have been tested, standard ranges have been made to have the correspondence Ct (q-PCR datum on the DNA extracted from the carpaccios)/CFU (experimental datum corresponding to the number of bacteria inoculated on the standard carpaccios) (for example, quantization of Hafnia alvei CIP57.31 by qPCR, see FIG. 1).

The following primers have been used:
for Serratia

QSF01-HAL-F:
(SEQ ID NO: 43)
TAACTTGGGAACTGCATTTGAAACTGGTC
QSF01-HAL-R:
(SEQ ID NO: 44)
CGCCACTGGTGTTCCTCCAGATC

for Hafnia alvei

QSF02-HAL-F:
(SEQ ID NO: 45)
ATTGGCAATTGGCGCTGAAGAAGGT
QSF02-HAL-R:
(SEQ ID NO: 46)
GCTCAGCCATATCAGCTGGGATC

for Brochothrix thermosphacta:

QSF01-BTH-F:
(SEQ ID NO: 47)
ACGAACGGATAAAGAGCTTGCTCTTTTG
QSF01-BTH-R:
(SEQ ID NO: 48)
CGAAACCGTCTTTCACTTGAACATCTTAT
QSF03-BTH-F:
(SEQ ID NO: 49)
GGACCAGAGGTTATCGAAACATTAACTG
QSF03-BTH-R:
(SEQ ID NO: 50)
TAATACCAGCAGCAGGAATTGCTT

for Weisella viridescens

QSF01-WVI-F:
(SEQ ID NO: 51)
TTGCTCAGATATGACGATGGACATTGCA
QSF01-WVI-R:
(SEQ ID NO: 52)
GACCATCTCTTAGTGATAGCAAGACCAT

for Pseudomonas

QSF01-PSD-F:
(SEQ ID NO: 53)
GGTCTTCGGATTGTAAAGCACTTTAAGTTG
QSF01-PSD-R:
(SEQ ID NO: 54)
GTAGTTAGCCGGTGCTTATTCTGTCG

for Leuconostoc

QSF01-LCN2-F:
(SEQ ID NO: 55)
GTGAAAGCCCGGAGCTCAACTC
QSF01-LCN2-R:
(SEQ ID NO: 56)
CTGGTGTTCTTCCACATATCTACGCATTC

for Lactobacillus sakei

QMF01-LSA-F:
(SEQ ID NO: 57)
CTGGCTATCCCGATACATACC
QMF01-LSA-R:
(SEQ ID NO: 58)
GCATATCTTGGCTACGGGCA
QMF07-LSA-F:
(SEQ ID NO: 59)
ATCTAGTAGACTCTGAATTTAAGGT
QMF07-LSA-R:
(SEQ ID NO: 60)
TTAACCGCTTCTAACACCTTTTGA
QMF15-LSA-F:
(SEQ ID NO: 61)
CTCTACCTCTGAAATGTCCAAG
QMF15-LSA-R:
(SEQ ID NO: 62)
TTTAGAGGTCTTCACCATTGTTG
QMF16-LSA-F:
(SEQ ID NO: 63)
ATTTATTCCAGCCAGCGTTTC
QMF16-LSA-R:
(SEQ ID NO: 64)
TGTGGAAAACATTCATGTACCG
QMF21-LSA-F:
(SEQ ID NO: 65)
AATTGGGTGCCTGCCGCG
QMF21-LSA-R:
(SEQ ID NO: 66)
GAATAAGAGCGCGAATACGG

### 3) Growth of Altering Species of the Carpaccio

FIG. 2 shows that the experimental field is of a great variability because the growth of each of the species varies from a carpaccio batch to another. The nature of the microbial ecosystem (qualitative and quantitative) naturally present on the food is one of the major factors which have an influence on this variability. On a set of several batches, it is possible to draw growth tendencies for each of the species: e.g. strong growth of about 3.5 Δ Log₁₀ CFU/g for Hafnia alvei or low growth of about 0.5 Δ Log₁₀ CFU/g for Weisseilla viridescens. This notion of global tendency will also be used for the effectiveness measurement of the composition.

### 4) Measurement of the Barrier Effect of the Compositions and of the Relative Influence of Each of the Strains within the Compositions

The effect of a composition can be directly measured by measuring Δ Log₁₀ CFU/g between T_{14 days} and T₀ for altering species.

It appears that the quality of the assembly of the strains is important to fight against alteration and that it is not sufficient to use a single strain of Lactobacillus sakei or to assemble randomly several strains to achieve a barrier effect.

### 5) Check of the Effectiveness of the Composition According to the Invention Against Alteration

The notion of the most or least effective composition is defined by the minimum or maximum value obtained by cumulating all the values of Δ Log₁₀ CFU/g for the altering bacteria. This arbitrary measurement enables the effect of a composition on all the altering species to be assessed. Thus, an active composition against Brochothrix thermosphacta can turn out to be poorly active against Leuconostocs.

Table 1 hereinafter shows the overall effectiveness of the composition according to the invention by comparison with the effectiveness of the 21 other compositions of L. sakei and controls without adding composition. The results are expressed in Δ Log₁₀ CFU/g for the target species.

TABLE 1

| | Enterobacteriaceae | Pseudomonadaceae | Leuconostocs | | |
|---|---|---|---|---|---|
| | (Serratia/ | (fragi/putida/ | (mesenteroides/ | | Brochothrix |
| | Hafnia) | fluorescens) | carnosum) | Hafnia alvei | thermosphacta |
| Not treated | 2.07 ± 1.16 | 2.18 ± 0.58 | 3.17 ± 0.83 | 4.01 ± 0.46 | 1.77 ± 0.66 |
| control^{a} | | | | | |
| Composition | 0.22 ± 0.68 | 0.13 ± 0.53 | 1.18 ± 0.46 | 2.91 ± 0.63 | 0.29 ± 0.47 |
| of the | | | | | |
| invention^{b} | | | | | |
| Other | 1.57 ± 0.99 | 0.43 ± 0.63 | 1.30 ± 0.32 | 3.98 ± 0.48 | 0.66 ± 0.92 |
| compositions^{c} | | | | | |

^{a}Control = 12 untreated carpaccio samples having a natural population of L. sakei
^{b}Composition of the invention = 6 carpaccio samples treated with the composition of the present invention
^{c}Other compositions = 21 carpaccio samples treated with 21 different cocktails of 4 strains of L. sakei

Table 1 shows, if the median values are considered, that:
the 21 tested compositions exhibited variable effects but that the median of the effects of these 21 compositions decreased relative to the average observed in the absence of any composition;
the composition according to the invention has a better performance than the most effective compositions among the other tested compositions. These results hold if the medians relative to the screening step and relative to the controls (without composition) are considered. The composition according to the present invention nearly abolishes Brochothrix thermosphacta growth, restricts very seriously that of Pseudomonas and restricts that of Hafnia alvei and the Serratia.

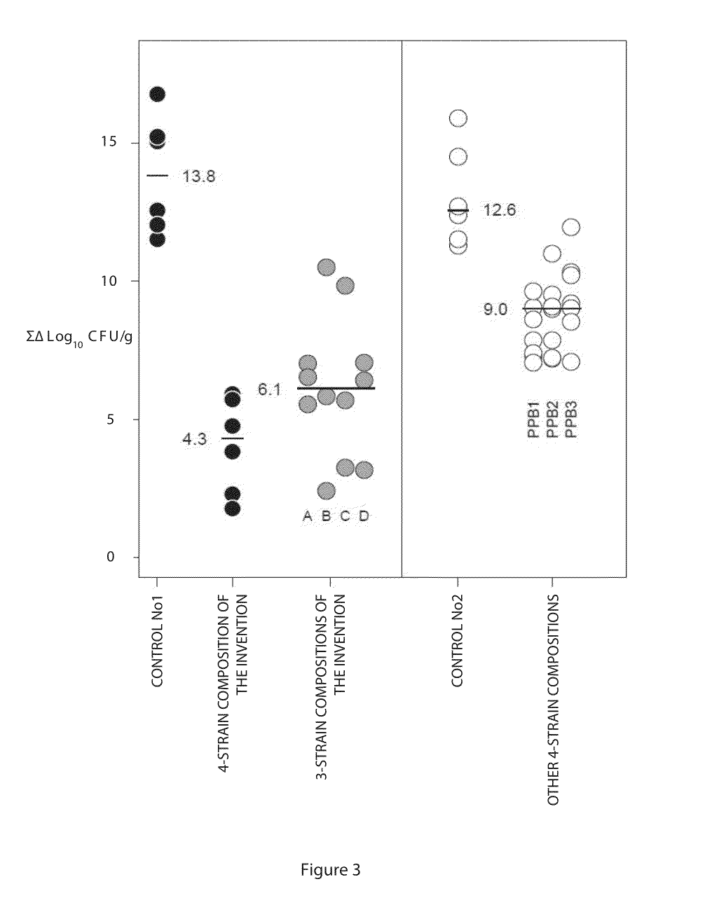

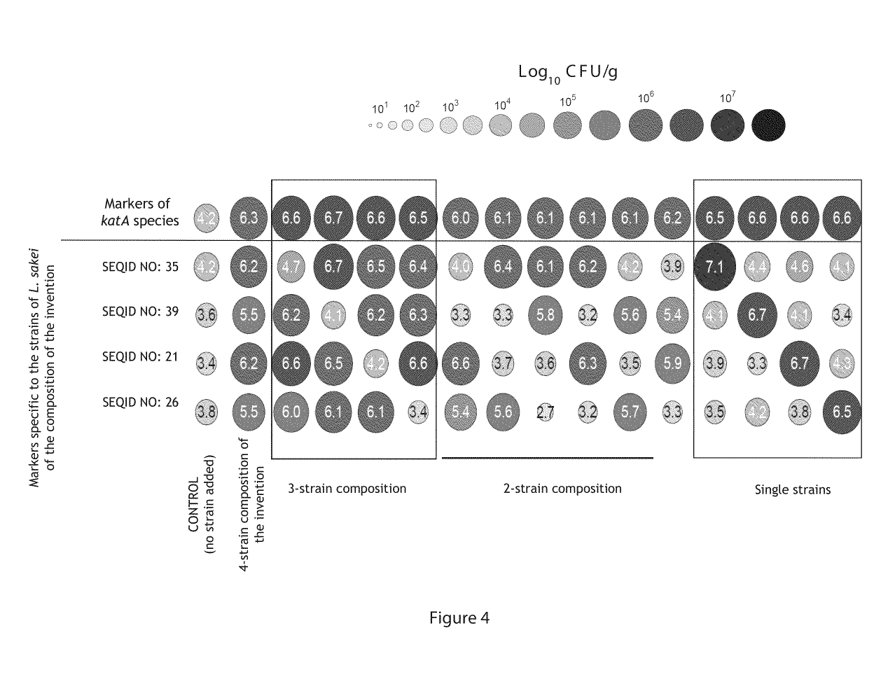

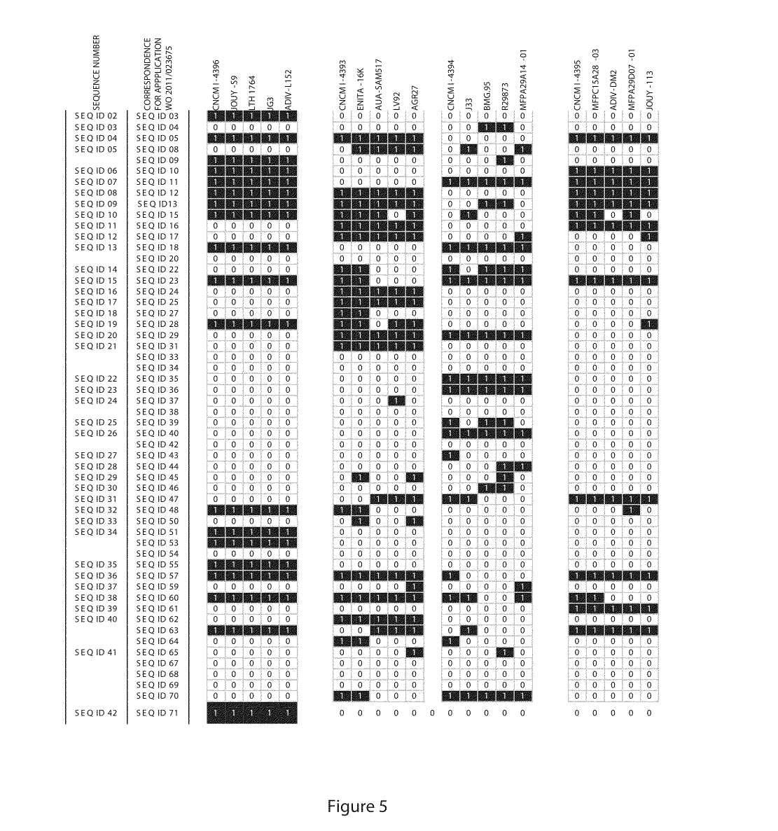

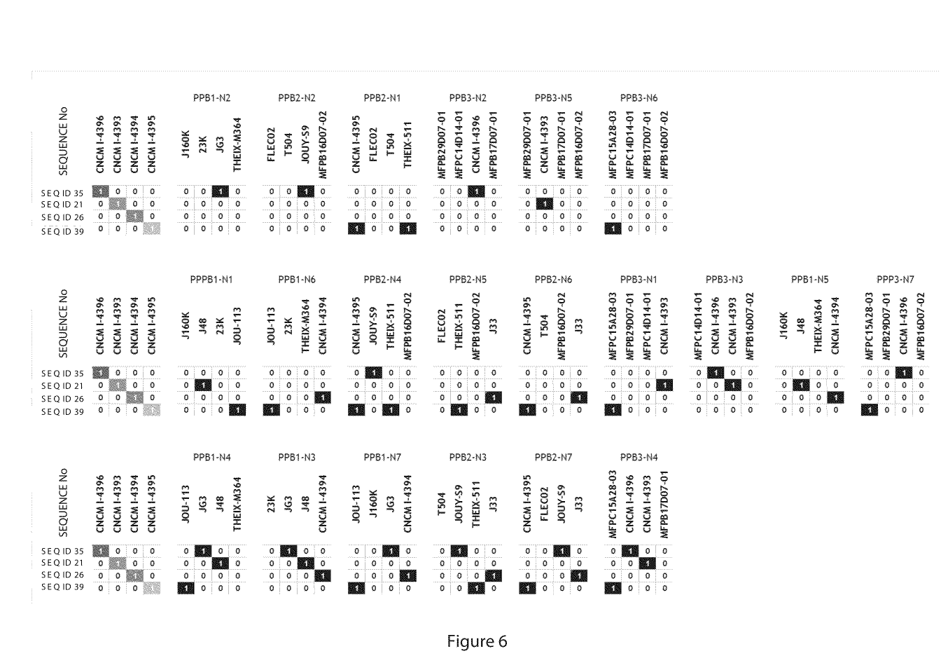

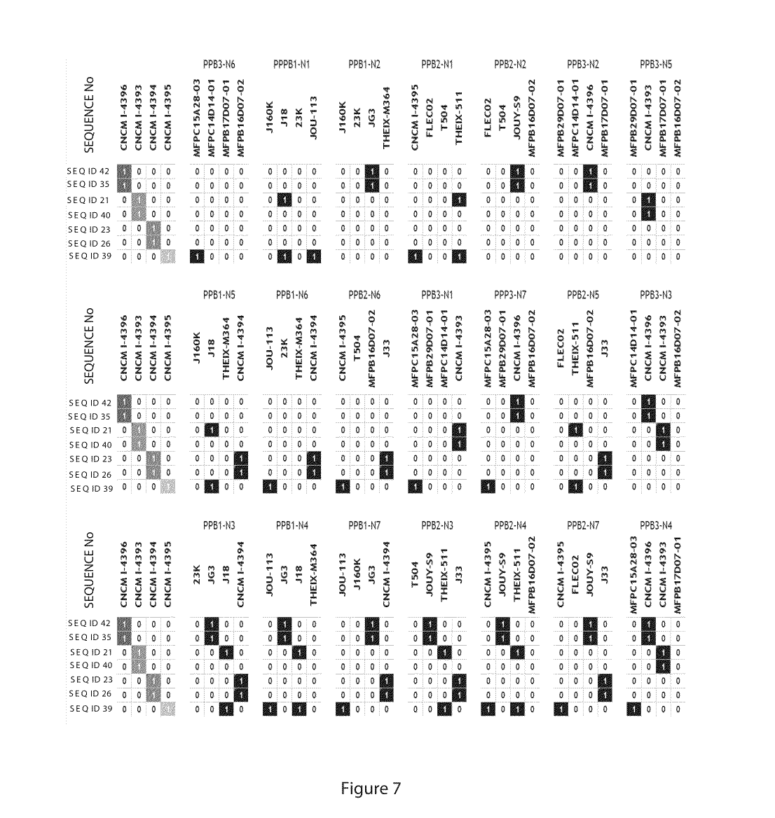

FIG. 3 shows a sharp overall decrease in the CGE between the control without composition (median on 6 batches of 13.8) and the control with the composition according to the present invention (median on 6 batches of 4.3). This composition is thus more effective than the best of the other tested compositions. The results on 3 batches of the different 3 combinations strains of the composition according to the invention show that these combinations are on average more effective that any other 4 strain combinations used during the screening step. A greater response amplitude is also observed with some 3 strain combinations than with the 4 strain composition. Finally, the stability of the effects on CGE seems to be more robust for the 4 strain composition than for the 3 strain combinations among the 4.

### 6) Identification of the Composition According to the Invention

The composition according to the invention has a single molecular imprinting, enabling molecular markers specific to each of the strains of the composition to be selected. An accurate quantization of the strains of the composition according to the present invention within a meat product as carpaccio is thus possible.

Marker katA (SEQ ID NO: 1), specific to the L. sakei species (present in all the strains, that is those of the composition according to the present invention but also those of the flora of L. sakei naturally present on meat) has been used as a positive control.

The markers of the sequences SEQ ID NO: 35, SEQ ID NO: 39, SEQ ID NO: 21, and SEQ ID NO: 26 are each specific of a single of the 4 strains making up the composition according to the present invention.

FIG. 4 shows that:
on the 14^{th} day (T14), without adding a composition (left column) the population of L. sakei is in the order of bout 10³-10⁴ CFU/g and is higher up to values in the order of about 5.10⁵-10⁶ CFU/g as soon as at least one of the strains of the composition has been inoculated;
the inoculated strains of L. sakei have been developed, however without reaching levels likely to be altering (the max value reached with the composition of the invention is 6.3 Log₁₀, and 6.6 Log₁₀ at 7.1 Log₁₀ CFU/g with 1-, 2- or 3-strain mixtures, which values are not considered as altering);
the use of marker katA enables an overall population of L. sakei which is more important to be revealed as soon as at least one strain of the composition is added (about 6 Log₁₀ CFU/g with strains addition versus about 4 Log₁₀ CFU/g without addition).
the strains have been developed each in a similar manner whether it is in the composition according to the invention, for the single strains or in the combinations of three or two strains from the four of the composition.
at T_{14 days} each strain of the composition is well quantifiable by virtue of its specific marker (in the combinations with one, two or three strains, it is easy to find each of the strains which has been inoculated (about 5.5-7 Log₁₀ CFU/g versus about 3 to 4 Log₁₀ CFU/g of background noise corresponding to the endogenous population of L. sakei).
therefore, the composition is actually identifiable and quantifiable.

1-11. (canceled)

12. A composition comprising at least three different strains of L. sakei bacteria, wherein:
said first strain of L. sakei bacteria comprises in its genome the following sequence:
SEQ ID NO: 35 or a sequence exhibiting at least 95% identity thereto,
said second strain of L. sakei bacteria comprises in its genome the following sequence:
SEQ ID NO: 21 or a sequence exhibiting at least 95% identity thereto,
said third strain of L. sakei bacteria comprises in its genome the following sequence:
SEQ ID NO: 26 or a sequence exhibiting at least 95% identity thereto.

13. The composition according to claim 12, wherein said first strain of L. sakei bacteria comprises in its genome the following sequences:
SEQ ID NO: 42, and
SEQ ID NO: 35 or a sequence exhibiting at least 95% identity thereto.

14. The composition according to claim 12, wherein said first strain of L. sakei bacteria comprises in its genome the following sequences:
SEQ ID NO: 2 or a sequence exhibiting at least 95% identity thereto,
SEQ ID NO: 34 or a sequence exhibiting at least 95% identity thereto,
SEQ ID NO: 42, and
SEQ ID NO: 35 or a sequence exhibiting at least 95% identity thereto;

15. The composition according to claim 12, wherein said first strain of L. sakei bacteria comprises in its genome the following sequences:
SEQ ID NO: 2 or a sequence exhibiting at least 95% identity thereto,
SEQ ID NO: 4 or a sequence exhibiting at least 95% identity thereto,
SEQ ID NO: 6 or a sequence exhibiting at least 95% identity thereto,
SEQ ID NO: 7 or a sequence exhibiting at least 95% identity thereto,
SEQ ID NO: 8 or a sequence exhibiting at least 95% identity thereto,
SEQ ID NO: 9 or a sequence exhibiting at least 95% identity thereto,
SEQ ID NO: 10 or a sequence exhibiting at least 95% identity thereto,
SEQ ID NO: 13 or a sequence exhibiting at least 95% identity thereto,
SEQ ID NO: 15 or a sequence exhibiting at least 95% identity thereto,
SEQ ID NO: 19 or a sequence exhibiting at least 95% identity thereto,
SEQ ID NO: 32 or a sequence exhibiting at least 95% identity thereto,
SEQ ID NO: 34 or a sequence exhibiting at least 95% identity thereto,
SEQ ID NO: 42,
SEQ ID NO: 35 or a sequence exhibiting at least 95% identity thereto,
SEQ ID NO: 36 or a sequence exhibiting at least 95% identity thereto, and
SEQ ID NO: 38 or a sequence exhibiting at least 95% identity thereto.

16. The composition according to claim 12, wherein said second strain of L. sakei bacteria comprises in its genome the following sequences:
SEQ ID NO: 21 or a sequence exhibiting at least 95% identity thereto, and
SEQ ID NO: 40 or a sequence exhibiting at least 95% identity thereto.

17. The composition according to claim 12, wherein said second strain of L. sakei bacteria comprises in its genome the following sequences:
SEQ ID NO: 16 or a sequence exhibiting at least 95% identity thereto,
SEQ ID NO: 17 or a sequence exhibiting at least 95% identity thereto,
SEQ ID NO: 21 or a sequence exhibiting at least 95% identity thereto, and
SEQ ID NO: 40 or a sequence exhibiting at least 95% identity thereto.

18. The composition according to claim 12, wherein said second strain of L. sakei bacteria comprises in its genome the following sequences:
SEQ ID NO: 4 or a sequence exhibiting at least 95% identity thereto,
SEQ ID NO: 8 or a sequence exhibiting at least 95% identity thereto,
SEQ ID NO: 9 or a sequence exhibiting at least 95% identity thereto,
SEQ ID NO: 11 or a sequence exhibiting at least 95% identity thereto,
SEQ ID NO: 12 or a sequence exhibiting at least 95% identity thereto,
SEQ ID NO: 16 or a sequence exhibiting at least 95% identity thereto,
SEQ ID NO: 17 or a sequence exhibiting at least 95% identity thereto,
SEQ ID NO: 20 or a sequence exhibiting at least 95% identity thereto,
SEQ ID NO: 21 or a sequence exhibiting at least 95% identity thereto,
SEQ ID NO: 36 or a sequence exhibiting at least 95% identity thereto,
SEQ ID NO: 38 or a sequence exhibiting at least 95% identity thereto,
SEQ ID NO: 40 or a sequence exhibiting at least 95% identity thereto.

19. The composition according to claim 12, wherein said second strain of L. sakei bacteria further comprises one of the sequences selected from the group consisting of the sequences: SEQ ID NO: 5, SEQ ID NO: 10, SEQ ID NO: 14, SEQ ID NO: 15, SEQ ID NO: 18, SEQ ID NO: 19, SEQ ID NO: 24, SEQ ID NO: 29, SEQ ID NO: 31, SEQ ID NO: 32, SEQ ID NO: 33, SEQ ID NO: 37, and SEQ ID NO: 41 or a sequence exhibiting at least 95% identity thereto respectively.

20. The composition according to claim 12, wherein said second strain of L. sakei bacteria further comprises from 2 to 13 of the sequences selected from the group consisting of the sequences: SEQ ID NO: 5, SEQ ID NO: 10, SEQ ID NO: 14, SEQ ID NO: 15, SEQ ID NO: 18, SEQ ID NO: 19, SEQ ID NO: 24, SEQ ID NO: 29, SEQ ID NO: 31, SEQ ID NO: 32, SEQ ID NO: 33, SEQ ID NO: 37, and SEQ ID NO: 41 or a sequence exhibiting at least 95% identity thereto respectively.

21. The composition according to claim 12, wherein said second strain of L. sakei bacteria further comprises from 7 to 12 of the sequences selected from the group consisting of the sequences: SEQ ID NO: 5, SEQ ID NO: 10, SEQ ID NO: 14, SEQ ID NO: 15, SEQ ID NO: 18, SEQ ID NO: 19, SEQ ID NO: 24, SEQ ID NO: 29, SEQ ID NO: 31, SEQ ID NO: 32, SEQ ID NO: 33, SEQ ID NO: 37, and SEQ ID NO: 41 or a sequence exhibiting at least 95% identity thereto respectively.

22. The composition according to claim 12, wherein said third strain of L. sakei bacteria comprises in its genome the following sequences:
SEQ ID NO: 23 or a sequence exhibiting at least 95% identity thereto, and
SEQ ID NO: 26 or a sequence exhibiting at least 95% identity thereto.

23. The composition according to claim 12, wherein said third strain of L. sakei bacteria comprises in its genome at least the following sequences:
SEQ ID NO: 22 or a sequence exhibiting at least 95% identity thereto,
SEQ ID NO: 23 or a sequence exhibiting at least 95% identity thereto, and
SEQ ID NO: 26 or a sequence exhibiting at least 95% identity thereto.

24. The composition according to claim 12, wherein said third strain of L. sakei bacteria comprises in its genome at least the following sequences:
SEQ ID NO: 7 or a sequence exhibiting at least 95% identity thereto,
SEQ ID NO: 13 or a sequence exhibiting at least 95% identity thereto,
SEQ ID NO: 15 or a sequence exhibiting at least 95% identity thereto,
SEQ ID NO: 20 or a sequence exhibiting at least 95% identity thereto,
SEQ ID NO: 22 or a sequence exhibiting at least 95% identity thereto,
SEQ ID NO: 23 or a sequence exhibiting at least 95% identity thereto, and
SEQ ID NO: 26 or a sequence exhibiting at least 95% identity thereto.

25. The composition according to claim 12, wherein said third strain of L. sakei bacteria further comprises one of the sequences selected from the group consisting of the sequences: SEQ ID NO: 3, SEQ ID NO: 5, SEQ ID NO: 9, SEQ ID NO: 12, SEQ ID NO: 14, SEQ ID NO: 25, SEQ ID NO: 27, SEQ ID NO: 28, SEQ ID NO: 29, SEQ ID NO: 30, SEQ ID NO: 31, SEQ ID NO: 36, SEQ ID NO: 37, SEQ ID NO: 38, and SEQ ID NO: 41 or a sequence exhibiting at least 95% identity thereto respectively.

26. The composition according to claim 12, wherein said third strain of L. sakei bacteria further comprises from 2 to 15 of the sequences selected from the group consisting of the sequences: SEQ ID NO: 3, SEQ ID NO: 5, SEQ ID NO: 9, SEQ ID NO: 12, SEQ ID NO: 14, SEQ ID NO: 25, SEQ ID NO: 27, SEQ ID NO: 28, SEQ ID NO: 29, SEQ ID NO: 30, SEQ ID NO: 31, SEQ ID NO: 36, SEQ ID NO: 37, SEQ ID NO: 38, and SEQ ID NO: 41 or a sequence exhibiting at least 95% identity thereto respectively.

27. The composition according to claim 12, wherein said third strain of L. sakei bacteria further comprises from 5 to 9 of the sequences selected from the group consisting of the sequences: SEQ ID NO: 3, SEQ ID NO: 5, SEQ ID NO: 9, SEQ ID NO: 12, SEQ ID NO: 14, SEQ ID NO: 25, SEQ ID NO: 27, SEQ ID NO: 28, SEQ ID NO: 29, SEQ ID NO: 30, SEQ ID NO: 31, SEQ ID NO: 36, SEQ ID NO: 37, SEQ ID NO: 38, and SEQ ID NO: 41 or a sequence exhibiting at least 95% identity thereto respectively.

28. The composition according to claim 12, further comprising at least a fourth strain of L. sakei different from said first, second and third strains.

29. The composition according to claim 28, wherein said fourth strain of L. sakei bacteria comprises in its genome at least the sequence SEQ ID NO: 39 or a sequence exhibiting at least 95% identity thereto.

30. The composition according to claim 28, wherein said fourth strain comprises in its genome at least the following sequences:
SEQ ID NO: 4 or a sequence exhibiting at least 95% identity thereto,
SEQ ID NO: 6 or a sequence exhibiting at least 95% identity thereto,
SEQ ID NO: 7 or a sequence exhibiting at least 95% identity thereto,
SEQ ID NO: 8 or a sequence exhibiting at least 95% identity thereto,
SEQ ID NO: 9 or a sequence exhibiting at least 95% identity thereto,
SEQ ID NO: 15 or a sequence exhibiting at least 95% identity thereto,
SEQ ID NO: 31 or a sequence exhibiting at least 95% identity thereto,
SEQ ID NO: 36 or a sequence exhibiting at least 95% identity thereto,
SEQ ID NO: 39 or a sequence exhibiting at least 95% identity thereto,

31. The composition according to claim 28, wherein said fourth strain further comprises one of the sequences selected from the group consisting of the sequences: SEQ ID NO: 10, SEQ ID NO: 12, SEQ ID NO: 19, SEQ ID NO: 32 and SEQ ID NO: 38 or a sequence exhibiting at least 95% identity thereto respectively.

32. The composition according to claim 28, wherein said fourth strain further comprises from 2 to 5 of the sequences selected from the group consisting of the sequences: SEQ ID NO: 10, SEQ ID NO: 12, SEQ ID NO: 19, SEQ ID NO: 32 and SEQ ID NO: 38 or a sequence exhibiting at least 95% identity thereto respectively.

33. The composition according to claim 28, wherein said fourth strain further comprises 2 of the sequences selected from the group consisting of the sequences: SEQ ID NO: 10, SEQ ID NO: 12, SEQ ID NO: 19, SEQ ID NO: 32 and SEQ ID NO: 38 or a sequence exhibiting at least 95% identity thereto respectively.

34. The composition according to claim 12 comprising at least the 4 strains of L. sakei deposited with the CNCM as the following numbers: CNCM I-4393, CNCM I-4394, CNCM I-4395, and CNCM I-4396.

35. A method for preserving an edible product, comprising the step of contacting said edible product with a composition as defined in claim 12.

36. The method of claim 35, wherein the edible product is a meat product.

37. The method of claim 35, wherein the edible product is beef meat.

38. The method of claim 35, wherein the edible product is chosen in the group consisting of beef carpaccio and chopped beef.

39. An edible product comprising a composition according to claim 12.
